(19) United States
(12) Patent Application Publication
(10) Pub. No.: US 2026/0364234 A1
(43) Pub. Date: Nov. 19, 2026

(54) MEDICAL FLUID GENERATION DEVICE

(71) Applicants: Vantive Health GmbH, Glattpark (Opfikon) (CH); Vantive US Healthcare LLC, Deerfield, IL (US)

(72) Inventors: Christian Vartia, Veberöd (SE); Henrik Lindgren, Genarp (SE); Per-Olof Borgqvist, Lund (SE)

(21) Appl. No.: 19/133,637

(22) PCT Filed: Nov. 28, 2023
(86) PCT No.: PCT/EP2023/083264

§ 371 Date: May 28, 2025

(30) Foreign Application Priority Data
Nov. 29, 2022 (SE) 2251390-7
Nov. 29, 2022 (SE) 2251391-5
Nov. 29, 2022 (SE) 2251392-3

(51) Int. Cl. A61M 1/14; A61M 1/16

(52) U.S. Cl. CPC A61M 1/154; A61M 1/153; A61M 1/1562; A61M 1/1607; A61M 1/1694

(57) ABSTRACT

A medical fluid generation apparatus is provided with a fluid circuit including a main fluid line having an inlet for receiving water, a pump active on the main fluid line to circulate fluid, a plurality of valves on the fluid circuit configurable to define different fluid paths for the fluid inside the fluid circuit, a supporting structure having a first primary container access for fluid communication with a primary concentrate container and a first auxiliary container access for fluid communication with an auxiliary concentrate container, a mixing container having an inlet and an outlet. The fluid circuit comprises a medical fluid outlet placed downstream the pump; a mixing container outlet line connecting the outlet of the mixing container to a first junction point on the main fluid line placed upstream the pump; a mixing container inlet line connecting a sixth junction point on the main fluid line downstream the pump to the inlet of the mixing container; a primary concentrate container outlet line connecting the first primary container access to a fifth junction point on the main fluid line upstream the; an auxiliary concentrate container outlet line connecting the first auxiliary container access to a third junction point on the main fluid line upstream the pump.

### BACKGROUND

The present disclosure relates generally to a medical fluid generation apparatus, in particular for dialysis fluid treatments. In more detail, embodiments of the present disclosure relate to batch preparation of medical fluid such as dialysis fluid for peritoneal dialysis or haemodialysis. In a further aspect, the present disclosure relates to a dry concentrate bag for medical fluid preparation.

Due to various causes, a person's renal system may fail. Renal failure produces several physiological derangements. It is no longer possible to balance water and minerals or to excrete daily metabolic load. Toxic end products of metabolism, such as, urea, creatinine, uric acid and others, may accumulate in a patient's blood and tissue.

Reduced kidney function and, above all, kidney failure is treated with dialysis. Dialysis removes waste, toxins and excess water from the body that normal functioning kidneys would otherwise remove. Dialysis treatment for replacement of kidney functions is critical to many people because the treatment is lifesaving.

One type of kidney failure therapy is Hemodialysis (“HD”), which in general uses diffusion to remove waste products from a patient's blood. A diffusive gradient occurs along the semi-permeable dialyzer between the blood and an electrolyte solution called dialysate or dialysis fluid to cause diffusion. HD fluids are typically created by the dialysis machines by mixing concentrates and clean water.

Hemofiltration (“HF”) is an alternative renal replacement therapy that relies on a convective transport of toxins from the patient's blood. HF is accomplished by adding substitution or replacement fluid to the extracorporeal circuit during treatment. The substitution fluid and the fluid accumulated by the patient in between treatments is ultrafiltered over the course of the HF treatment, providing a convective transport mechanism that is particularly beneficial in removing middle and large molecules.

Hemodiafiltration (“HDF”) is a treatment modality that combines convective and diffusive clearances. HDF uses dialysis fluid flowing through a dialyzer, similar to standard hemodialysis, to provide diffusive clearance. In addition, substitution solution is delivered directly to the extracorporeal circuit, providing convective clearance. Here, more fluid than the patient's excess fluid is removed from the patient, causing the increased convective transport of waste products from the patient. The additional fluid removed is replaced via the substitution or replacement fluid.

Another type of kidney failure therapy is peritoneal dialysis (“PD”), which infuses a dialysis solution, also called dialysis fluid, into a patient's peritoneal cavity via a catheter. The dialysis fluid is in contact with the peritoneal membrane in the patient's peritoneal cavity. Waste, toxins, and excess water pass from the patient's bloodstream, through the capillaries in the peritoneal membrane, and into the dialysis fluid due to diffusion and osmosis, i.e., an osmotic gradient occurs across the membrane. An osmotic agent in the PD dialysis fluid provides the osmotic gradient. Used or spent dialysis fluid is drained from the patient, removing waste, toxins, and excess water from the patient. This cycle is repeated, e.g., multiple times. PD fluids are typically prepared in a factory and shipped to the patient's home in ready-to-use bags.

There are various types of peritoneal dialysis therapies, including continuous ambulatory peritoneal dialysis (“CAPD”), automated peritoneal dialysis (“APD”), tidal flow dialysis and continuous flow peritoneal dialysis (“CFPD”). CAPD is a manual dialysis treatment, where fluid transport is driven by gravity. If initially full of used dialysis fluid, the patient manually connects an implanted catheter to a drain to allow the used or spent dialysis fluid to drain from the patient's peritoneal cavity. The patient then switches fluid communication so that the patient catheter communicates with a bag of fresh dialysis fluid to infuse the fresh dialysis fluid through the catheter and into the patient. The patient disconnects the catheter from the fresh dialysis fluid bag and allows the dialysis fluid to dwell within the peritoneal cavity, wherein the transfer of waste, toxins, and excess water takes place. After a dwell period, the patient repeats the manual dialysis procedure, for example, four times per day. If the peritoneal cavity is not initially full of used dialysis fluid, the sequence is instead fill, dwell, and drain. Manual peritoneal dialysis requires a significant amount of time and effort from the patient, leaving ample room for improvement.

Automated peritoneal dialysis (“APD”) is similar to CAPD in that the dialysis treatment includes drain, fill, and dwell cycles. APD machines, however, perform the cycles automatically, typically while the patient sleeps. APD machines free patients from having to manually perform the treatment cycles and from having to transport supplies during the day. APD machines are fluidly connected to an implanted catheter, to a source or bag of fresh dialysis fluid and to a fluid drain. APD machines pump fresh dialysis fluid from a dialysis fluid source, through the catheter and into the patient's peritoneal cavity. APD machines also allow for the dialysis fluid to dwell within the chamber and for the transfer of waste, toxins, and excess water to take place. The source may include multiple liters of dialysis fluid including several solution bags. APD machines pump used or spent dialysate from the peritoneal cavity, through the catheter, and to the drain. As with the manual process, several drain, fill and dwell cycles occur during dialysis. A “last fill” may occur at the end of the APD treatment. The last fill fluid may remain in the peritoneal cavity of the patient until the start of the next treatment, or may be manually emptied at some point during the day.

As indicated, the dialysis machines, either for haemodialysis or for peritoneal dialysis, usually requires an important amount of fresh dialysis liquid to be used during the respective therapies. Handling a huge number of relatively heavy bags (e.g., ⅝ liter bags) storing ready to use dialysis fluid is sometimes problematic and requires efforts and storing location. This is particularly, but not exclusively, relevant in case of home dialysis, wherein it is the patient or his/her family members who has to handle the fluid reservoirs. A need exists accordingly to provide an improved system capable producing and possibly temporary storing a certain amount of medical fluid starting from fresh (pre-treated) water and proper concentrates.

Some prior documents disclose preparation of dialysis fluid from dry concentrates, mainly for HD. Also, the general concept of mixing PD-fluid from concentrates is known.

EP1312386A2 discloses a bag with dry concentrate, for example an acid concentrate; the system is intended for preparation of dialysis fluid for blood dialysis. The bag holds a diluted concentrate solution and has two separate ports. Water is pumped into the bag via one port, whereby the dry concentrate is dissolved. After the bag is filled with a certain amount of water, the diluted solution is circulated to ensure it is dissolved. Thereafter the fluid may be pumped to a dialysis apparatus for use.

From U.S. Pat. No. 10,195,329B2 it is also known to mix dry concentrate (bicarbonate) with water and use conductivity measurements to determine if the concentrate is dissolved properly. The system prepares dialysate solution online for dialysis.

From US2019/0060850A1 it is known to have a container with concentrates and mix it with water by recirculation. The medical system, intended to produce dialysis solution for hemodialysis, produces a solution batch directly.

From EP2305331A1 it is known to dissolve powder in two different containers and circulate it in a circulation line. When the powder is dissolved, it is dosed with flow chambers and the conductivity is checked.

US2020/0390956A1 describes a PD machine for preparing PD-solution from RO water.

EP3222303B1 illustrates mixing of powder with water and mixing a dialysis solution from drug A and drug B into a first container, recirculating, and delivering it to a second container from where it is sent to a dialysis device for use.

US2019262522A1 disclose to mix osmotic agent, electrolyte, and water in a mixing container for mixing liquid concentrates into PD fluid.

### SUMMARY

The present disclosure sets forth systems and methods for producing a medical fluid, particularly a dialysis fluid, including peritoneal dialysis (“PD”) fluid and hemodialysis (“HD”) fluids. While the present disclosure focuses more on PD fluid, the teachings discussed herein is also applicable to other treatment and injectable fluids, such as continuous renal replacement treatment (“CRRT”) fluids including HD fluids, substitution or replacement fluids for hemofiltration (“HF”) and hemodiafiltration (“HDF”), lactated ringers and the like. The medical fluid generation apparatus receives water purified by a water purification apparatus (e.g., a reverse osmosis device) or, alternatively, receives water that is firstly purified inside the apparatus and then used to prepare a suitable dialysis fluid. In a PD fluid example, the pure water is mixed with PD concentrates when making PD fluid and is stored in a mixing container or bag; usually two PD concentrates are used including electrolyte, osmotic agent (such as glucose) and buffer concentrates. In a HD fluid example, pure water is mixed with one or two concentrates, such as an A concentrate and a B concentrate, including a buffer concentrate (e.g., bicarbonate, acetate or lactate) and including an electrolyte concentrate with sodium, calcium, magnesium and potassium ions, and is stored in a mixing container or bag.

In light of the disclosure set forth herein, and without limiting the disclosure in any way, in a 1^{st} independent aspect, which may be combined with any other aspect or portion thereof described herein, a medical fluid generation apparatus (1) comprises:
a fluid circuit (14) including a main fluid line (4) having an inlet point (5) for receiving water;
a pump (8) configured to operate on the main fluid line (4) to circulate fluid at least in the main fluid line (4);
at least one of:
   a primary concentrate container (2) having at least one port (6, 7) in fluid communication with the main fluid line (4);
   an auxiliary concentrate container (3) having at least one port (9, 10) in fluid communication with the main fluid line (4);
a mixing container (11) having an inlet (12) in fluid communication with the main fluid line (4) and an outlet (13) in fluid communication with the main fluid line (4);
a plurality of valves configured to operate on the fluid circuit (14) configurable to define different fluid paths for the fluid inside the fluid circuit (14),
a control unit (24) configured to operate said pump (8) and said plurality of valves.

In a 2^{nd} independent aspect, a medical fluid generation apparatus (1) is provided comprising:
a fluid circuit (14) including a main fluid line (4) having an inlet point (5) for receiving water;
a pump (8) configured to operate on the main fluid line (4) to circulate fluid at least in the main fluid line (4);
a plurality of valves configured to operate on the fluid circuit (14) configurable to define different fluid paths for the fluid inside the fluid circuit (14),
a supporting structure (42) housing the fluid circuit (14), the pump (8) and the plurality of valves, and comprising:
   optionally an entrance (5a) for water, in particular said entrance (5a) defining the inlet point (5) for receiving water;
   a first primary container access (43, 44) being part of the fluid circuit (14) for fluid communication with a primary concentrate container (2), the first primary container access (43, 44) being in fluid communication with the main fluid line (4); and
   a first auxiliary container access (45, 46) being part of the fluid circuit (14) for fluid communication with an auxiliary concentrate container (3), the first auxiliary container access (45, 46) being in fluid communication with the main fluid line (4);
a control unit (24) configured to operate said pump (8) and said plurality of valves,
   wherein the medical fluid generation apparatus further comprises at least one of:
a mixing container access (47) being part of the fluid circuit (14) and a mixing container exit (48) being part of the fluid circuit (14) for fluid communication with respectively an inlet (12) of a mixing container (11) and an outlet (13) of the mixing container (11), the mixing container access (47) and the mixing container exit (48) being respectively in fluid communication with the main fluid line (4);
a mixing container (11) having an inlet (12) in fluid communication with the main fluid line (4) and an outlet (13) in fluid communication with the main fluid line (4),
   wherein the fluid circuit (14) comprises:
a medical fluid outlet (27) to provide medical fluid and a medical fluid outlet line (28) for carrying medical fluid to the medical fluid outlet (27), the medical fluid outlet line (28) being configured for receiving fluid from the main fluid line (4) and being placed downstream the pump (8);
a mixing container outlet line (16) connecting the outlet (13) of the mixing container (11) to a mixing container outlet junction point or first junction point (j1) on the main fluid line (4), the first junction point (j1) being placed upstream the pump (8);
a mixing container inlet line (15) connecting a sixth junction point (j6) on the main fluid line (4) to the inlet (12) of the mixing container (11), the sixth junction point (j6) being placed downstream the pump (8);
a primary concentrate container outlet line (32; 36) connecting the first primary container access (43, 44) to a primary concentrate container junction point (j4; j5) on the main fluid line (4), the primary concentrate container junction point (j4; j5) being placed upstream the pump (8);
an auxiliary concentrate container outlet line (40; 41) connecting the first auxiliary container access (45, 46) to an auxiliary concentrate container junction point (j2; j3) on the main fluid line (4), wherein the auxiliary concentrate container junction point (j2; j3) being placed upstream the pump (8).

The provision of a main fluid line (4) where the pump (8) is configured to operate and of specifically placed junction points for the mixing container inlet and outlet lines straddling the pump and of junction points for the primary concentrate container outlet line and the primary concentrate container outlet line upstream the pump allow to create a fluid path that may use one single pump for all the fluid generation steps. The various fluids always move along the main fluid line (4) controlled by the pump (8) that only works in a forward direction (i.e., it is never necessary to reverse its pumping along the main line). This fluid circuit outline also allows to create proper recirculating paths that may be used to e.g., dissolve dry concentrates, diluting liquid concentrates and/or homogenize the prepared mixtures.

In a 3^{rd} aspect depending on the previous aspect, the medical fluid generation apparatus further comprises a conductivity meter (25) associated with the control unit (24) configured to determine a conductivity of the fluid circulating in the medical fluid generation apparatus, wherein the conductivity meter (25) is connected to the main fluid line (4) downstream the mixing container outlet junction point (j1), the auxiliary concentrate container junction point (j2; j3), and the primary concentrate container junction point (j4; j5) and upstream the mixing container inlet junction point (j6) and, optionally, upstream the pump (8).

In a 4^{th} independent aspect, a medical fluid generation apparatus (1) is provided comprising:
a fluid circuit (14) including a main fluid line (4) having an inlet point (5) for receiving water;
a pump (8) configured to operate on the main fluid line (4) to circulate fluid at least in the main fluid line (4);
a plurality of valves configured to operate on the fluid circuit (14) configurable to define different fluid paths for the fluid inside the fluid circuit (14),
a supporting structure (42) housing the fluid circuit (14), the pump (8) and the plurality of valves, the supporting structure (42) comprising an entrance defining the inlet point (5) for receiving water and at least one of:
   a first primary container access (43) and a second primary container access (44) part of the fluid circuit (14) for fluid communication with a primary concentrate container (2), the first primary container access (43) and the second primary container access (44) being in fluid communication with the main fluid line (4);
   a first auxiliary container access (45) and a second auxiliary container access (46) part of the fluid circuit (14) for fluid communication with an auxiliary concentrate container (3), the first auxiliary container access (45) and the second auxiliary container access (46) being in fluid communication with the main fluid line (4);
a control unit (24) configured to operate said pump (8) and said plurality of valves,
   wherein the medical fluid generation apparatus further comprises at least one of:
a mixing container access (47) being part of the fluid circuit (14) and a mixing container exit (48) part of the fluid circuit (14) for fluid communication with respectively an inlet (12) of a mixing container (11) and an outlet (13) of the mixing container (11), the mixing container access (47) and the mixing container exit (48) being respectively in fluid communication with the main fluid line (4);
a mixing container (11) having an inlet (12) in fluid communication with the main fluid line (4) and an outlet (13) in fluid communication with the main fluid line (4),
   wherein said fluid paths comprise at least:
a mixing recirculation path (R1), wherein the plurality of valves are configured to allow a recirculation of the fluid contained in the mixing container (11) through: the outlet (13), at least one portion of the main fluid line (4) on which the pump (8) is configured to operate, and the inlet (12);
a concentrate recirculation path (Z6; X5), wherein the plurality of valves are configured to allow a recirculation of the fluid contained in one of the primary concentrate container (2) and the auxiliary concentrate container (3) through: one of the first primary container access (43) and the first auxiliary container access (45), at least one portion of the main fluid line (4) on which the pump (8) is configured to operate, and the respective one of the second primary container access (44) and the second auxiliary container access (46).

In a 5^{th} independent aspect, a medical fluid generation apparatus (1) is provided comprising:
a fluid circuit (14) including a main fluid line (4) having an inlet (5) for receiving water;
a pump (8) configured to operate on the main fluid line (4) to circulate fluid at least in the main fluid line (4);
a plurality of valves configured to operate on the fluid circuit (14) configurable to define different fluid paths for the fluid inside the fluid circuit (14),
a supporting structure (42) housing the fluid circuit (14), the pump (8) and the plurality of valves, the supporting structure (42) comprising at least one of:
   a primary container access (43; 44) being part of the fluid circuit (14) for fluid communication with a primary concentrate container (2), the primary container access (43; 44) being in fluid communication with the main fluid line (4);
   an auxiliary container access (45; 46) being part of the fluid circuit (14) for fluid communication with an auxiliary concentrate container (3), the auxiliary container access (45; 46) being in fluid communication with the main fluid line (4);
wherein the medical fluid generation apparatus further comprises at least one of:
a mixing container access (47) part of the fluid circuit (14) and a mixing container exit (48) part of the fluid circuit (14) for fluid communication with respectively an inlet (12) of a mixing container (11) and an outlet (13) of the mixing container (11), the mixing container access (47) and the mixing container exit (48) being respectively in fluid communication with the main fluid line (4);
a mixing container (11) having an inlet (12) in fluid communication with the main fluid line (4) and an outlet (13) in fluid communication with the main fluid line (4),
a sensor (52) connected to the main fluid line (4) to measure a property of the fluid flowing in the main fluid line (4);
a control unit (24) configured to operate said pump (8) and said plurality of valves, and to receive signals from the sensor (52) related to said property of the fluid flowing in the main fluid line (4);
   wherein said fluid paths comprise a mixing recirculation path (R1) including the outlet (13), at least one portion of the main fluid line (4) on which the pump (8) is configured to operate, and the inlet (12), wherein the control unit (24) is configured to control the plurality of valves to set the mixing recirculation path (R1) and to allow a recirculation of the fluid contained in the mixing container (11) through the mixing recirculation path (R1);
   wherein the control unit (24) is further configured to:
      recirculate the fluid in the mixing recirculation path (R1);
      detect a time-resolved variation of said property of the fluid recirculating in the mixing recirculating path (R1), said time-resolved variation of said property of the fluid being related to homogeneity of the fluid recirculating in the mixing recirculating path (R1);
      interrupt the recirculation step upon the time-resolved variation meeting a variation criterion.

In a 6^{th} independent aspect, a medical fluid generation apparatus (1) is provided comprising:
a fluid circuit (14) including a main fluid line (4) having an inlet point (5) for receiving water;
a pump (8) configured to operate on the main fluid line (4) to circulate fluid at least in the main fluid line (4);
a plurality of valves configured to operate on the fluid circuit (14) configurable to define different fluid paths for the fluid inside the fluid circuit (14),
a supporting structure (42) housing the fluid circuit (14), the pump (8) and the plurality of valves, the supporting structure (42) comprising an entrance defining the inlet point (5) for receiving water and at least one of:
   a first primary container access (43, 44) being part of the fluid circuit (14) for fluid communication with a primary concentrate container (2), the first primary container access (43, 44) being in fluid communication with the main fluid line (4);
   a first auxiliary container access (45, 46) being part of the fluid circuit (14) for fluid communication with an auxiliary concentrate container (3), the first auxiliary container access (45, 46) being in fluid communication with the main fluid line (4);
a control unit (24) configured to operate said pump (8) and said plurality of valves, wherein the medical fluid generation apparatus (1) further comprises at least one of:
a mixing container access (47) being part of the fluid circuit (14) and a mixing container exit (48) part of the fluid circuit (14) for fluid communication with respectively an inlet (12) of a mixing container (11) and an outlet (13) of the mixing container (11), the mixing container access (47) and the mixing container exit (48) being respectively in fluid communication with the main fluid line (4);
a mixing container (11) having an inlet (12) in fluid communication with the main fluid line (4) and an outlet (13) in fluid communication with the main fluid line (4).

In a 7^{th} aspect according to aspect 6, the medical fluid generation apparatus further comprises at least one of (and preferably both):
a primary concentrate container (2) having at least one port (6, 7) in fluid communication with the main fluid line (4), said port (6, 7) being in fluid communication with the first primary container access (43, 44);
an auxiliary concentrate container (3) having at least one port (9, 10) in fluid communication with the main fluid line (4), said port (9, 10) being in fluid communication with the first auxiliary container access (45, 46).

In an 8^{th} aspect, according to any one of aspects 6 and 7, wherein the inlet (12) of the mixing container (11) is in fluid communication with the mixing container access (47) and the outlet (13) of the mixing container (11) is in fluid communication with the mixing container exit (48).

In a 9^{th} independent aspect, a medical fluid generation apparatus (1) is provided comprising:
a fluid circuit (14) including a main fluid line (4) having an inlet point (5) for receiving water;
a pump (8) configured to operate on the main fluid line (4) to circulate fluid at least in the main fluid line (4);
a primary concentrate container (2) having at least one of: a first port (6) in fluid communication with the main fluid line (4) and a second port (7) in fluid communication with the main fluid line (4);
an auxiliary concentrate container (3) having a first port (9) in fluid communication with the main fluid line (4) and a second port (10) in fluid communication with the main fluid line (4);
a mixing container (11) having an inlet (12) in fluid communication with the main fluid line (4) and an outlet (13) in fluid communication with the main fluid line (4);
a plurality of valves configured to operate on the fluid circuit (14) configurable to define different fluid paths for the fluid inside the fluid circuit (14),
a control unit (24) configured to operate said pump (8) and said plurality of valves,
wherein said fluid paths comprise at least:
   a mixing recirculation path (R1), wherein the plurality of valves are configured to allow a recirculation of the fluid contained in the mixing container (11) through: the outlet (13), at least one portion of the main fluid line (4) on which the pump (8) is configured to operate, and the inlet (12);
   a concentrate recirculation path (Z6, X5), wherein the plurality of valves are configured to allow a recirculation of the fluid contained in one of the primary concentrate container (2) and the auxiliary concentrate container (3) through: the first port (6; 9), at least one portion of the main fluid line (4) on which the pump (8) is configured to operate, and the second port (7; 10).

In a 10^{th} independent aspect, a method of generating a medical fluid with a medical fluid generation apparatus (1) is provided, the apparatus comprising:
a fluid circuit (14) including a main fluid line (4) having an inlet (5) for receiving water;
a pump (8) configured to operate on the main fluid line (4) to circulate fluid at least in the main fluid line (4);
a plurality of valves configured to operate on the fluid circuit (14) configurable to define different fluid paths for the fluid inside the fluid circuit (14),
a supporting structure (42) housing the fluid circuit (14), the pump (8) and the plurality of valves, the supporting structure (42) comprising at least one of:
   a first primary container access (43) and a second primary container access (44) being part of the fluid circuit (14) for fluid communication with a primary concentrate container (2), the first primary container access (43) and the second primary container access (44) being in fluid communication with the main fluid line (4);
   a first auxiliary container access (45) and a second auxiliary container access (46) being part of the fluid circuit (14) for fluid communication with an auxiliary concentrate container (3), the first auxiliary container access (45) and the second auxiliary container access (46) being in fluid communication with the main fluid line (4);
a control unit (24) configured to operate said pump (8) and said plurality of valves,
   wherein the medical fluid generation apparatus (1) further comprises at least one of:
a mixing container access (47) being part of the fluid circuit (14) and a mixing container exit (48) being part of the fluid circuit (14) for fluid communication with respectively an inlet (12) of a mixing container (11) and an outlet (13) of the mixing container (11), the mixing container access (47) and the mixing container exit (48) being respectively in fluid communication with the main fluid line (4);
a mixing container (11) having an inlet (12) in fluid communication with the main fluid line (4) and an outlet (13) in fluid communication with the main fluid line (4), the method comprising:
   controlling the plurality of valves to set a mixing recirculation path (R1) in the fluid circuit, the mixing recirculation path (R1) including the outlet (13), at least one portion of the main fluid line (4) on which the pump (8) is configured to operate, and the inlet (12),
   recirculating the fluid contained in the mixing container (11) through the mixing recirculation path (R1);
   controlling the plurality of valves to set a concentrate recirculation path (Z6; X5) in the fluid circuit, the concentrate recirculation path (Z6; X5) including one of the first primary container access (43) and the first auxiliary container access (45), at least one portion of the main fluid line (4) on which the pump (8) is configured to operate, and the respective one of the second primary container access (44) and the second auxiliary container access (46),
   recirculating the fluid contained in one of the primary concentrate container (2) and the auxiliary concentrate container (3) through the concentrate recirculation path (Z6; X5).

In an 11^{th} independent aspect, a method of generating a medical fluid with a medical fluid generation apparatus (1) is provided, the apparatus comprising:
a fluid circuit (14) including a main fluid line (4) having an inlet (5) for receiving water;
a pump (8) configured to operate on the main fluid line (4) to circulate fluid at least in the main fluid line (4);
a plurality of valves configured to operate on the fluid circuit (14) configurable to define different fluid paths for the fluid inside the fluid circuit (14),
a supporting structure (42) housing the fluid circuit (14), the pump (8) and the plurality of valves, the supporting structure (42) comprising at least one of:
   a primary container access (43; 44) being part of the fluid circuit (14) for fluid communication with a primary concentrate container (2), the primary container access (43; 44) being in fluid communication with the main fluid line (4);
   an auxiliary container access (45; 46) being part of the fluid circuit (14) for fluid communication with an auxiliary concentrate container (3), the auxiliary container access (45; 46) being in fluid communication with the main fluid line (4);
wherein the medical fluid generation apparatus (1) further comprises at least one of:
a mixing container access (47) part of the fluid circuit (14) and a mixing container exit (48) part of the fluid circuit (14) for fluid communication with respectively an inlet (12) of a mixing container (11) and an outlet (13) of the mixing container (11), the mixing container access (47) and the mixing container exit (48) being respectively in fluid communication with the main fluid line (4);
a mixing container (11) having an inlet (12) in fluid communication with the main fluid line (4) and an outlet (13) in fluid communication with the main fluid line (4),
a sensor (52) connected to the main fluid line (4) to measure a property of the fluid flowing in the main fluid line (4);
a control unit (24) configured to operate said pump (8) and said plurality of valves, and to receive signals from the sensor (52) related to said property of the fluid flowing in the main fluid line (4);
   the method comprising:
      configuring a mixing recirculation path (R1) in the fluid circuit, the mixing recirculation path (R1) including the outlet (13), at least one portion of the main fluid line (4) on which the pump (8) is configured to operate, and the inlet (12);
      recirculating the fluid in the mixing recirculation path (R1);
      detecting a time-resolved variation of said property of the fluid recirculating in the mixing recirculating path (R1), said time-resolved variation of said property of the fluid being related to homogeneity of the fluid recirculating in the mixing recirculating path (R1);
      interrupting the recirculation step upon the time-resolved variation meeting a variation criterion.

In the following aspects, the method steps for independent method aspects 10 and 11 are performed by the control unit of the apparatus.

In a 12^{th} aspect according to any one of the previous aspects, the fluid circuit (14) comprises a mixing container inlet line (15) connecting a sixth junction point (j6) on the main fluid line (4) to the inlet (12) of the mixing container (11), said plurality of valves comprising a mixing container inlet valve (VMI) configured to operate on the mixing container inlet line (15).

In a 13^{th} aspect according to the previous aspect the sixth junction point (j6) is placed downstream the pump (8).

In a 14^{th} aspect according to any one of the previous aspects, the apparatus further comprises a pressure sensor (P2) configured for sensing a pressure of the fluid in the main fluid line (4) downstream the pump (8).

In a 15^{th} aspect according to the previous aspect, when depending on aspect 13, the pressure sensor (P2) is placed on the mixing container inlet line (15), optionally between the sixth junction point (j6) and the mixing container inlet valve (VMI).

In a 16^{th} aspect according to any one of the previous aspects, the fluid circuit (14) comprises a mixing container outlet line (16) connecting the outlet (13) of the mixing container (11) to a first junction point (j1) on the main fluid line (4), said plurality of valves comprising a mixing container outlet valve (VMO) configured to operate on the mixing container outlet line (16).

In an 17^{th} aspect according to the previous aspect, the first junction point (j1) is placed upstream the pump (8).

In an 18^{th} aspect according to any one of the previous aspects, the apparatus further comprises an auxiliary pressure sensor (P1) configured for sensing a pressure occurring on the main fluid line (4) upstream the pump (8).

In a 19^{th} aspect according to the previous aspect, when depending on aspect 16, the auxiliary pressure sensor (P1) is placed on the mixing container outlet line (16), optionally between the first junction point (j1) and the mixing container outlet valve (VMO).

In a 20^{th} aspect according to any one of the previous aspects, the apparatus further comprises a sensor (17) for providing information relating to the volume of fluid contained in the mixing container (11), in particular wherein said sensor (17) is chosen in the group consisting of a weight scale and a level sensor.

In a 21^{st} aspect according to any one of the previous aspects, the apparatus further comprises at least one sterilization device (18) configured to operate on a fluid volume contained into the mixing container (11), said sterilization device (18) being for example a UV sterilization device, wherein in particular the sterilization device (18) is placed inside the mixing container (11), for example located on a bottom wall (11a) of the mixing container (11).

In a 22^{nd} aspect according to any one of the previous aspects, the apparatus further comprises one or more sterilization devices configured to operate on the fluid contained into the fluid circuit (14), said one or more sterilization devices being for example UV sterilization devices, wherein in particular said one or more sterilization devices being configured to operate on the main line (4) and/or the mixing container inlet line (15) and/or the mixing container outlet line (16).

In a 23^{rd} aspect according to any one of the previous aspects, the mixing container (11) further comprises a vent (19) including a vent line (20) to selectively expel excess air, optionally a filter (21) to filter any air passing through the vent line (20) and/or an air valve (22) to selectively block air passage through the vent line (20) being placed along the vent line (20), in particular the air valve (22) being interposed on the vent line (20) between the mixing chamber (11) and the filter (21).

In a 24^{th} aspect according to the previous aspect, the vent line (20) is connected to an air drain.

In 25^{th} aspect according to any one of the previous aspects, the mixing container (11) is a closed container having either:
exclusively the inlet (12) and the outlet (13) placing an inner volume in fluid communication with an outside; or,
exclusively the inlet (12), the outlet (13) and a vent (19) placing an inner volume in fluid communication with an outside, wherein the vent (19) is placed on a top portion (11b) of the mixing container (11).

In a 26^{th} aspect according to any one of the previous aspects, the mixing container (11) is a non-removable and/or durable (non-disposable) container.

In a 26^{th} bis aspect according to any one of the previous aspects, the fluid circuit (14) is durable. A durable fluid circuit (14) may be disinfected or sterilized when proper and then reused for a subsequent medical fluid preparation. It simplifies the user operation for readying the apparatus and reduces the waste since no disposable circuit is to be dressed onto the device and subsequently discarded.

In a 27^{th} aspect according to any one of the previous aspects, the mixing container (11) is a removable and/or disposable container, in particular both inlet (12) and outlet (13) of the mixing container (11) being removably connected to the main fluid line (4), optionally both inlet (12) and outlet (13) of the mixing container (11) including self-sealing valves to prevent fluid from exiting the mixing container once separated from the medical fluid generation apparatus.

In a 28^{th} aspect according to the previous aspect, the mixing container (11) is a flexible bag.

In a 29^{th} aspect according to any one of the previous aspects, the apparatus further comprises a heater (23) controlled by the control unit (24) and configured to heat the fluid circulating in the medical fluid generation apparatus, said heater being placed on the main fluid line (4).

In a 30^{th} aspect according to any one of the previous aspects, the apparatus further comprises at least one sensor (52) placed on the main fluid line (4) and configured to measure a property of the fluid flowing in the main fluid line (4), the control unit (24) being configured to receive signals from the sensor (52) related to said property of the fluid flowing in the main fluid line (4). In particular, the property of the fluid flowing in the main fluid line (4) is chosen in the group consisting of a conductivity of the fluid flowing in the main fluid line (4), a concentration of a substance in the fluid flowing in the main fluid line (4), a concentration dependent physical properties, such as sound velocity, viscosity, density, a temperature of the fluid flowing in the main fluid line (4), and an optical property of the fluid flowing in the main fluid line (4) and combinations thereof.

In a 31^{st} aspect according to the previous aspect, the apparatus comprises two sensors (52, 53) placed in series with each other on the main fluid line (4) and both configured to measure the property of the fluid flowing in the main fluid line (4), the control unit (24) being configured to receive signals from the sensors (52, 53).

In a 32^{nd} aspect according to any one of the previous aspects, the apparatus further comprises a conductivity meter (25) associated with the control unit (24) configured to determine a conductivity of the fluid circulating in the medical fluid generation apparatus, in particular the conductivity meter (25) being placed on the main fluid line (4) downstream the first junction point (j1), the third junction point (j3), and the fifth junction point (j5) and upstream the sixth junction point (j6) and, optionally, the pump (8).

In a 33^{rd} aspect according to the previous aspect, the apparatus further comprises a second conductivity meter (26) associated with the control unit (24) configured to independently determine said conductivity of the fluid circulating in the medical fluid generation apparatus, both said conductivity meter (25) and second conductivity meter (26) being configured to send independent signals to the control unit (24) configured to determine the conductivity of the same fluid using the conductivity measurement from the second conductivity meter (26), said second conductivity meter (26) being placed on the main fluid line (4), downstream the first junction point (j1), the third junction point (j3), and the fifth junction point (j5) and upstream the sixth junction point (j6) and, optionally, the pump (8).

In a 34^{th} aspect depending on the previous aspects 29 and 32, the heater (23) is placed upstream the pump (8) and/or the conductivity meter (25). For example, the conductivity meter (25) is interposed between the heater (23) and the pump (8).

In a 34^{th} bis aspect according to aspects 30 and 32, the at least one sensor (52) is the conductivity meter (25). In particular, according to aspect 31 and 32, the second sensor (53) is the second conductivity meter (26).

In a 35^{th} aspect according to any one of the previous aspects, the apparatus further comprises a temperature sensor (T1) for sensing the temperature of the fluid in the main fluid line (4), in particular the temperature sensor (T1) is placed downstream the first junction point (j1), the third junction point (j3), and the fifth junction point (j5) and upstream the sixth junction point (j6) and, optionally, the pump (8).

In a 36^{th} aspect according to the previous aspect, the temperature sensor (T1) is either an external sensor or a sensor integrated in a conductivity meter (25; 26).

In a 37^{th} aspect according to the previous aspect and twenty-second aspect, the control unit (24) is configured to receive the temperature signal from the temperature sensor (T1) and to control the heater (23) at least based on the received temperature sensor (T1).

The warming of fluid is desired to get at stable conductivity reading from the conductivity meter (25). The conductivity meter (25) is usually configured for a certain temperature, or small temperature interval, where it measures the conductivity accurately. This temperature may be 37 degrees C to always avoiding the need to cool the fluid (e.g., if the room temp is 30 °C and the set temperature to measure conductivity is 21 °C, then fluid temperature would need to be cooled). Further, the fluid shall anyway be warmed to 37 degrees before patient entry (however, this warming may in some embodiment be made by a downstream located heater-not shown).

In a 38^{th} aspect according to any one of the previous aspects, the apparatus further comprises an auxiliary temperature sensor (T2) for sensing the temperature of the fluid in the main fluid line (4), in particular the temperature sensor (T2) is placed downstream the first junction point (j1), the third junction point (j3), and the fifth junction point (j5) and upstream the sixth junction point (j6) and, optionally, the pump (8). The auxiliary temperature sensor (T2) is either an external sensor or a sensor integrated in a conductivity meter (25; 26).

In a 39^{th} aspect according to any one of the previous aspects, the pump (8) is a volumetric pump.

In a 40^{th} aspect according to any one of the previous aspects, the support structure (42) further comprises a medical fluid outlet (27) to provide medical fluid to a connected medical apparatus and the fluid circuit (14) comprises a medical fluid outlet line (28) for carrying medical fluid to the medical fluid outlet (27), said plurality of valves comprising a medical fluid outlet valve (VFO) configured to operate on the medical fluid outlet line (28).

In a 41^{st} aspect according to the previous aspect, when depending on aspect 6, wherein a first end of the medical fluid outlet line (28) is connected to the mixing container inlet line (15) and a second end of the medical fluid outlet line (28) is connected to the medical fluid outlet (27), wherein optionally the first end is connected upstream the mixing container inlet valve (VMI) to receive the medical fluid flowing in a portion of the mixing container inlet line (15) upstream the mixing container inlet valve (VMI).

In a 42^{nd} aspect according to any one of the previous two aspects, the medical fluid outlet line (28) is configured for or receives fluid from the main fluid line (4) and is placed downstream the pump (8).

In a 43^{rd} aspect according to any one of the previous aspects, the fluid circuit (14) further comprises a drain port (29) to discard fluid to a drain and a drain outlet line (30) carrying the fluid from the main line (4) to the drain port (29), said plurality of valves further comprising a drain valve (VDR) configured to operate on the drain outlet line (30).

In a 44^{th} aspect according to any one of the previous aspects, the fluid circuit (14) comprises a primary concentrate container inlet line (31) connecting a junction point, in particular the sixth junction point (j6), on the main fluid line (4) to the first primary container access (43, 44), said plurality of valves comprising a primary concentrate container inlet valve (VGI) configured to operate on the primary concentrate container inlet line (31).

In a 45^{th} aspect according to the previous aspect, the sixth junction point (j6) is placed downstream the pump (8).

In a 46^{th} aspect according to any one of the previous aspects, when depending on aspects 4, 9 or 10, the medical apparatus further comprises a concentration sensor (SG) configured for sensing a concentration of at least one substance in the fluid flowing in the concentrate recirculation path.

In a 47^{th} aspect according to the previous aspect, when depending on aspect 31, the concentration sensor (SG) is placed on the primary concentrate container inlet line (31), optionally between the primary concentrate container inlet valve (VGI) and the first primary container access (43).

In a 48^{th} aspect according to any one of the previous aspects, the fluid circuit (14) comprises a primary concentrate container outlet line (32) connecting the second primary container access (44) to a fifth junction point (j5) on the main fluid line (4), said plurality of valves comprising a primary concentrate container outlet valve (VGO) configured to operate on the primary concentrate container outlet line (32).

In a 49^{th} aspect according to the previous aspect, the fifth junction point (j5) is placed upstream the pump (8).

In a 50^{th} aspect according to any one of the previous aspects, the medical apparatus further comprises a de-aeration chamber (33) configured for allowing air removal and placed on the primary concentrate container outlet line (32) between the first primary container access (43, 44) and the primary concentrate container outlet valve (VGO), the de-aeration chamber (33) comprising a vent (34) including a vent line (35) to selectively expel excess air, optionally a gas valve (VGA) configured to operate on the vent line (35) to selectively block air passage through such vent line (35), in particular the gas valve (VGA) being interposed on the vent line (35) between the primary concentrate container (2) and a drain.

In a 51^{st} aspect according to the previous aspect, the medical apparatus further comprises a level sensor associated with the de-aeration chamber (33) to check a fluid level in the de-aeration chamber (33).

In a 52^{nd} aspect according to any one of the previous two aspects, when depending on aspect 48, the de-aeration chamber (33) is placed on the primary concentrate container outlet line (32), optionally between the second port (7) and the primary concentrate container outlet valve (VGO).

In a 53^{rd} aspect according to any one of the previous aspects, the medical apparatus further comprises an auxiliary de-aeration chamber (51) configured for allowing air removal and placed on the auxiliary concentrate container outlet line (40) between the first auxiliary container access (45, 46) and the auxiliary concentrate container outlet valve (VAO), the auxiliary de-aeration chamber (51) comprising a vent (34) including a vent line (35a) to selectively expel excess air, optionally an auxiliary gas valve (VGA2) configured to operate on the vent line (35a) to selectively block air passage through such vent line (35a), in particular the auxiliary gas valve (VGA2) being interposed on the vent line (35a) between the auxiliary concentrate container (3) and a drain.

In a 54^{th} aspect according to the previous aspect, the medical apparatus further comprises a level sensor associated with the auxiliary de-aeration chamber (51) to check a fluid level in the auxiliary de-aeration chamber (51).

In a 55^{th} aspect according to any one of the previous aspects, the fluid circuit (14) comprises a first concentrate mixing line (36) connecting a fourth junction point (j4) on the main fluid line (4) to the first primary container access (43, 44), particularly to an intermediate point (31a) of the primary concentrate container inlet line (31), said plurality of valves comprising a first concentrate mixing valve (VGM) configured to operate on the first concentrate mixing line (36), wherein the fourth junction point (j4) is placed upstream the pump (8).

In a 56^{th} aspect according to the previous aspect, the connection to the intermediate point (31a) of the primary concentrate container inlet line (31) is proximate to the first primary container access (43).

In a 57^{th} aspect according to any one of the previous aspects, the medical apparatus further comprising a dry concentrate housed in the primary concentrate container (2) in the form of a powder or a granulate.

In a 58^{th} aspect according to the previous aspect, the dry concentrate includes an osmotic agent, particularly glucose monohydrate.

In a 59^{th} aspect according to any one of the previous two aspects, the dry concentrate comprises at least 80% of glucose monohydrate and particularly 100% of glucose monohydrate.

In a 60^{th} aspect according to any one of the previous aspects, the primary concentrate container (2) is a flexible bag comprising two plastic films welded together to define a housing space for the concentrate.

In a 61^{st} aspect according to any one of the previous aspects, the primary concentrate container (2) is a dry concentrate bag for preparing a dialysis fluid comprising:
two plastic films welded together along welding lines to define a housing space for the dry concentrate;
a dry concentrate housed within the housing space;
an inlet portion comprising an inlet port for receiving a fluid, the inlet port being placed in correspondence of a bottom area in use conditions of the dry concentrate bag;
an outlet portion comprising an outlet port for letting the fluid mixed with the dry concentrate to leave the housing space, the outlet port being placed in correspondence of a top area in use conditions of the dry concentrate bag.

In a 62^{nd} aspect according to the previous aspect, the inlet portion comprises a first inclined welding line and a second inclined welding line both emerging from the inlet port and defining a lower cone portion of the housing space, the inlet port being placed at the lowest point of the lower cone portion.

In a 63^{rd} aspect according to any one of the previous two aspects, the outlet portion comprises a third inclined welding line and a fourth inclined welding line both converging to the outlet port and defining an upper cone portion of the housing space, the outlet port being placed at the highest point of the upper cone portion.

In a 64^{th} aspect according to any one of the previous aspects, the fluid circuit (14) comprises an auxiliary concentrate container inlet line (37) connecting a junction point, in particular the sixth junction point (j6), on the main fluid line (4) to the first auxiliary container access (45, 46), said plurality of valves comprising an auxiliary concentrate container inlet valve (VAI) configured to operate on the auxiliary concentrate container inlet line (37).

In a 65^{th} aspect according to the previous aspect, the sixth junction point (j6) is placed downstream the pump (8).

In a 66^{th} aspect according to any one of the previous two aspects, the auxiliary concentrate container inlet line (37) comprises a common tract (38) with a primary concentrate container inlet line (31), said common tract (38) starting at the junction point, in particular the sixth junction point (j6), and arriving at a branch (39) where the primary concentrate container inlet line (31) and the auxiliary concentrate container inlet line (37) separate.

In a 67^{th} aspect according to the previous aspect, the common tract (38) does not include any valve.

In a 68^{th} aspect according to any one of the previous two aspects, the auxiliary concentrate container inlet valve (VAI) is configured to operate downstream said branch (39).

In a 69^{th} aspect according to any one of the previous two aspects, the concentrate container inlet valve (VGI) is configured to operate on the primary concentrate container inlet line (31) downstream said branch (39).

In a 70^{th} aspect according to any one of the previous aspects, the fluid circuit (14) comprises an auxiliary concentrate container outlet line (40) connecting the second port (10) of the auxiliary concentrate container (3) to a third junction point (j3) on the main fluid line (4), said plurality of valves comprising an auxiliary concentrate container outlet valve (VAO) configured to operate on the auxiliary concentrate container outlet line (40).

In a 71^{st} aspect according to the previous aspect, the third junction point (j3) is placed upstream the pump (8).

In a 72^{nd} aspect according to any one of the previous aspects, the fluid circuit (14) comprises a second concentrate mixing line (41) connecting a second junction point (j2) on the main fluid line (4) to the first auxiliary container access (45, 46), particularly to an intermediate point (37a) of the auxiliary concentrate container inlet line (37), said plurality of valves comprising a second concentrate mixing valve (VAM) configured to operate on the second concentrate mixing line (41), wherein second junction point (j2) is placed upstream the pump (8).

In a 73^{rd} aspect according to the previous aspect, connection to the intermediate point (37a) at the auxiliary concentrate container inlet line (37) is proximate to the first auxiliary container access (45.

In a 74^{th} aspect according to any one of the previous aspects, the auxiliary concentrate container (3) comprises liquid concentrate (LC′), in particular electrolyte and/or buffer.

In a 75^{th} aspect according to any one of the previous aspects, the main fluid line (4) comprises a plurality among a first junction point (j1), a second junction point (j2), a third junction point (j3), a fourth junction point (j4), a fifth junction point (j5), a respective fluid line of the fluid circuit (14) departing from each junction point (j1; j2; j3; j4; j5), the pump (8) being downstream said plurality of junction points, wherein the second junction point (j2) is placed upstream the third junction point (j3) on the main fluid line (4); and/or the fourth junction point (j4) is placed upstream the fifth junction point (j5) main fluid line (4); and/or the first junction point (j1) is placed upstream each other junction point (j2; j3; j4; j5).

In a 76^{th} aspect according to the previous aspect, said plurality of valves comprises a water inlet valve (VWI) placed on the main fluid line (4) upstream said plurality of junction points (j1; j2; j3; j5).

In a 77^{th} aspect according to any one of the previous aspects, the control unit (24) is configured to control the pump (8) to pump fluid inside the main line (4) in a forward direction from upstream the pump where the inlet point (5) for receiving water, the first junction point (j1), the third junction point (j3), and the fifth junction point (j5) are placed and to pump fluid inside the main line (4) in a forward direction towards downstream the pump where the sixth junction point (j6) is placed, the control unit (24) is not configured to control the pump (8) to pump the fluid in the main line (4) in reverse direction from the sixth junction point (j6) towards any of the inlet point (5) for receiving water, the first junction point (j1), the third junction point (j3), and the fifth junction point (j5) during steps for medical fluid generation.

In a 78^{th} aspect according to any one of the previous aspects, the apparatus is configured for preparing dialysis fluid, in particular PD fluid or HD fluid.

In a 79^{th} aspect according to any one of the previous aspects, the medical apparatus further comprises a water purification equipment configured to provide pure water to inlet point (5).

The water purification equipment may be an external device feeding pure water to the entrance for water 5a or, alternatively, an inner device that receives water to be purified from the entrance 5a and feeds pure water to the inlet point 5a. In the latter configuration, the water purification equipment is built inside the supporting structure (42).

In an 80^{th} aspect according to the previous aspect, the water purification equipment includes a central water purification station configured to feed the inlet point (5) or a standalone water purifier configured to feed the inlet point (5).

In an 81^{st} aspect according to any one of the previous aspects, said fluid paths comprise a mixing recirculation path (R1) comprising the mixing container (11), the outlet (13), a mixing container outlet line (16) connecting the outlet (13) of the mixing container (11) to a first junction point (j1) on the main fluid line (4), the at least one portion of the main fluid line (4) on which the pump (8) is configured to operate from the first junction point (j1) to a sixth junction point (j6), a mixing container inlet line (15) connecting the sixth junction point (j6) on the main fluid line (4) to the inlet (12) of the mixing container (11), and the inlet (12).

In an 82^{nd} aspect according to the previous aspect, the mixing recirculation path (R1) comprises the mixing container outlet valve (VMO) configured to operate on the mixing container outlet line (16) and the mixing container inlet valve (VMI) configured to operate on the mixing container inlet line (15), when the control unit (24) controls the plurality of valves to set the mixing recirculation path, the mixing container outlet valve (VMO) and the mixing container inlet valve (VMI) are configured to allow fluid to recirculate in the mixing recirculation path, in particular the other valves of the plurality of valves configured to operate on the fluid circuit (14) that are configurable to let fluid enter into or exit from the mixing recirculation path being closed, optionally all the other valves of the plurality of valves configured to operate on the fluid circuit (14) different from the mixing container outlet valve (VMO) and the mixing container inlet valve (VMI) being closed.

In an 83^{rd} aspect according to any one of the previous aspects, said fluid paths comprise a first concentrate recirculation path (Z6) comprising the primary concentrate container (2), the second port (7), a primary concentrate container outlet line (32) connecting the second port (7) of the primary concentrate container (2) to a fifth junction point (j5) on the main fluid line (4), the at least one portion of the main fluid line (4) on which the pump (8) is configured to operate extending from the fifth junction point (j5) to a sixth junction point (j6), a primary concentrate container inlet line (31) connecting the sixth junction point (j6) on the main fluid line (4) to the first port (6) of the primary concentrate container (2), and the first port (6).

In an 84^{th} aspect according to any one of the previous aspects, the concentrate recirculation path (Z6; X5) includes a first concentrate recirculation path (Z6) and the primary concentrate container (2) has a first port (6) and a second port (7) each in fluid communication with the main fluid line (4), said first port (6) being in fluid communication with the first primary container access (43), said second port (7) being in fluid communication with the second primary container access (44), wherein the first concentrate recirculation path (Z6) comprises the primary concentrate container (2), the second port (7), the second primary container access (44), a primary concentrate container outlet line (32) connecting the second primary container access (44) to a fifth junction point (j5) on the main fluid line (4), the at least one portion of the main fluid line (4) on which the pump (8) is configured to operate extending from the fifth junction point (j5) to a sixth junction point (j6), a primary concentrate container inlet line (31) connecting the sixth junction point (j6) on the main fluid line (4) to the first primary container access (43), and the first port (6) of the primary concentrate container (2).

In an 85^{th} aspect according to the previous aspect, the first concentrate recirculation path (Z6) comprises a primary concentrate container outlet valve (VGO) configured to operate on the primary concentrate container outlet line (32) and a primary concentrate container inlet valve (VGI) configured to operate on the primary concentrate container inlet line (31), when the control unit (24) controls the plurality of valves to set the first concentrate recirculation path, the primary concentrate container outlet valve (VGO) and the primary concentrate container inlet valve (VGI) are configured to allow fluid to recirculate in the first concentrate recirculation path, in particular the other valves of the plurality of valves configured to operate on the fluid circuit (14) that are configurable to let fluid enter into or exit from the first concentrate recirculation path being closed, optionally all the other valves of the plurality of valves configured to operate on the fluid circuit (14) different from the primary concentrate container outlet valve (VGO) and the primary concentrate container inlet valve (VGI) being closed.

In an 86^{th} aspect according to any one of the previous aspects, said fluid paths comprise an auxiliary concentrate recirculation path (X5), wherein the plurality of valves are configured to allow a recirculation of the fluid contained in the other one of the primary concentrate container (2) and the auxiliary concentrate container (3) through: the first port (6; 9), at least one portion of the main fluid line (4) on which the pump (8) is configured to operate, and the second port (7; 10).

In an 87^{th} aspect according to the previous aspect, said auxiliary concentrate recirculation path (X5) comprises the auxiliary concentrate container (3), the second port (10), an auxiliary concentrate container outlet line (40) connecting the second port (10) of the auxiliary concentrate container (3) to a third junction point (j3) on the main fluid line (4), the at least one portion of the main fluid line (4) on which the pump (8) is configured to operate extending from the third junction point (j3) to a sixth junction point (j6), an auxiliary concentrate container inlet line (37) connecting the sixth junction point (j6) on the main fluid line (4) to the first port (9) of the auxiliary concentrate container (3), and the first port (9).

In an 88^{th} aspect according to any one of the previous aspects, said fluid paths comprise an auxiliary concentrate recirculation path (X5), wherein the plurality of valves are configured to allow a recirculation of the fluid contained in the other one of the primary concentrate container (2) and the auxiliary concentrate container (3) through: the other one of the first primary container access (43) and the first auxiliary container access (45), at least one portion of the main fluid line (4) on which the pump (8) is configured to operate, and the respective other one of the second primary container access (44) and the second auxiliary container access (46).

In an 89^{th} aspect according to the previous aspect, the auxiliary concentrate container (3) has a first port (9) and a second port (10) each in fluid communication with the main fluid line (4), said first port (9) being in fluid communication with the first auxiliary container access (45), said second port (10) being in fluid communication with the second auxiliary container access (46), wherein said auxiliary concentrate recirculation path (X5) comprises the auxiliary concentrate container (3), the second port (10), the second auxiliary container access (46), an auxiliary concentrate container outlet line (40) connecting the second auxiliary container access (46) to a third junction point (j3) on the main fluid line (4), the at least one portion of the main fluid line (4) on which the pump (8) is configured to operate extending from the third junction point (j3) to a sixth junction point (j6), an auxiliary concentrate container inlet line (37) connecting the sixth junction point (j6) on the main fluid line (4) to the first auxiliary container access (45), and the first port (9) of the auxiliary concentrate container (3).

In a 90^{th} aspect according to any one of the previous aspects 87 to 89, the auxiliary concentrate recirculation path (X5) comprises an auxiliary concentrate container outlet valve (VAO) configured to operate on the auxiliary concentrate container outlet line (40) and an auxiliary concentrate container inlet valve (VAI) configured to operate on the auxiliary concentrate container inlet line (37), when the control unit (24) controls the plurality of valves to set the second concentrate recirculation path, the auxiliary concentrate container outlet valve (VAO) and the auxiliary concentrate container inlet valve (VAI) are configured to allow fluid to recirculate in the second concentrate recirculation path, in particular the other valves of the plurality of valves configured to operate on the fluid circuit (14) that are configurable to let fluid enter into or exit from the second concentrate recirculation path being closed, optionally all the other valves of the plurality of valves configured to operate on the fluid circuit (14) different from the auxiliary concentrate container outlet valve (VAO) and the auxiliary concentrate container inlet valve (VAI) being closed.

In a 91^{st} aspect according to any one of the previous aspects, the control unit (24) is configured to operate the plurality of valves to set the different fluid paths for the fluid inside the fluid circuit (14) and at least:
said mixing recirculation path (R1); and
said concentrate recirculation path (Z6, X5), particularly said first concentrate recirculation path (Z6) and/or said auxiliary concentrate recirculation path (X5);
   in particular wherein the control unit (24) is configured to first set said concentrate recirculation path (Z6, X5) and subsequently to set said mixing recirculation path (R1).

In a 92^{nd} aspect according to any one of the previous aspects, the control unit (24) is configured to run a dry concentrate dissolution procedure including sending a dissolution liquid, e.g., water, to at least one of the primary concentrate container (2) and the auxiliary concentrate container (3), to dissolve a dry concentrate and form a liquid concentrated mixture.

In a 94^{th} aspect according to the previous aspect, the dry concentrate dissolution procedure includes heating the dissolution liquid and/or the concentrated mixture by means of a heater (23) optionally connected to the main fluid line (4), wherein in particular the dissolution liquid is heated up to a temperature sufficient to achieve complete dissolution in a certain time period, for example for glucose the dissolution liquid is heated up to at least 60 °C and more specifically up to at least 70 °C and the concentrated mixture is maintained to at least 40 °C and more specifically up to at least 50 °C.

In a 95^{th} aspect according to any one of the previous two aspects, the dry concentrate dissolution procedure includes a concentrate mixture recirculation step in which the control unit (24) is configured to set the concentrate recirculation path (Z6, X5) and to recirculate the concentrated mixture to homogenize.

In a 96^{th} aspect according to any one of the previous three aspects, the dry concentrate dissolution procedure includes one or more of:
an air evacuation step;
a priming step of the main fluid line (4);
a recirculation path priming step;
a dissolution liquid fill step, optionally comprising first dissolution liquid fill step and a second dissolution liquid fill step;
a concentrate mixture recirculation step; and
a concentration test step;
   wherein the control unit is configured to execute the dry concentrate dissolution procedure which optionally includes at least the air evacuation step, the dissolution liquid fill step and the concentrate mixture recirculation step, the control unit (24) executing said three steps in a time sequence starting from the air evacuation step and ending with the concentrate mixture recirculation step.

In a 97^{th} aspect according to the previous aspect, the control unit (24) is configured to execute the air evacuation step by controlling the plurality of valves to set an air evacuation flow path (Z1) to evacuate air from the primary concentrate container (2) and driving the pump (8) to remove air from the primary concentrate container (2).

In a 98^{th} aspect according to the previous aspect, in the air evacuation flow path (Z1) the plurality of valves are configured to allow air contained in the primary concentrate container (2) to flow into a primary concentrate container outlet line (32) and into a de-aeration chamber (33) under the action of the pump (8), optionally a primary concentrate container outlet valve (VGO) acting on the primary concentrate container outlet line (32) and a drain valve (VDR) acting on a drain outlet line (30) being open, in particular the other valves of the plurality of valves, configured to operate on the fluid circuit (14) and that are configurable to let fluid enter into or exit from the air evacuation flow path (Z1), being closed, optionally all the other valves of the plurality of valves configured to operate on the fluid circuit (14) different from the primary concentrate container outlet valve (VGO) and the drain valve (VDR) being closed.

In a 99^{th} aspect according to any one of the previous two aspects, during the air evacuation step the control unit (24) is configured to operate the pump (8) at a pre-set pumping rate for a pre-fixed time period and stops the pump (8) after said pre-fixed time period has lapsed. Notably, pumping is performed until a predefined low level in the deaeration chamber is ensured.

In a 100^{th} aspect according to any one of the previous four aspects, the control unit (24) is configured to execute the priming step of the main fluid line (4) by controlling the plurality of valves to set a main fluid line prime path (Z2) and by controlling the pump (8) to prime the main fluid line (4), particularly after the air evacuation step.

In a 101^{st} aspect according to the previous aspect, in the main fluid line prime path (Z2) the plurality of valves are configured to allow a dissolution liquid, e.g., water, to enter into the main fluid line (4) and flow towards a drain outlet line (30) under the action of the pump (8), optionally a water inlet valve (VWI) connected to the main fluid line (4) and a drain valve (VDR) connected to a drain outlet line (30) being open, in particular the other valves of the plurality of valves configured to operate on the fluid circuit (14) that are configurable to let fluid enter into or exit from the main fluid line prime path (Z2) being closed, optionally all the other valves of the plurality of valves configured to operate on the fluid circuit (14) different from the water inlet valve (VWI) and the drain valve (VDR) being closed.

In a 102^{nd} aspect according to any one of the previous two aspects, during the priming step of the main fluid line (4) the control unit (24) is configured to operate the pump (8) at a pre-set pumping rate up to a pre-fixed pumping volume and to stop the pump (8) after said pre-fixed pumping volume has been reached. The pump (8) is operated until a prefixed volume greater than the main fluid line volume from water inlet 5a to sixth junction point j6 is achieved (indeed air may be present between water inlet and fourth junction point j4 e.g., at the start in case of an empty and dry system). Optionally, a safety factor z (e.g. 1.5-2) is considered to the main fluid line volume to ensure that no air remains between the fourth junction point j4 and the sixth junction point j6.

In a 103^{rd} aspect according to any one of the previous seven aspects, the control unit (24) is configured to execute the recirculation path priming step by controlling the plurality of valves to set a recirculation priming path (Z3) and by controlling the pump (8) to prime the recirculation priming path (Z3), particularly after the air evacuation step and/or after the priming step of the main fluid line (4).

In a 104^{th} aspect according to the previous aspect, in the recirculation priming path (Z3) the plurality of valves are configured to allow fluid circulation from a sixth junction point (j6) on the main fluid line (4) towards a primary concentrate container inlet line (31), into a first concentrate mixing line (36) to a fourth junction point (j4) on the main fluid line (4), in at least a portion of the main fluid line (4) on which the pump (8) is configured to operate extending to the sixth junction point (j6), optionally a primary concentrate container inlet valve (VGI) connected to the primary concentrate container inlet line (31) and a first concentrate mixing valve (VGM) connected to the first concentrate mixing line (36) being open, in particular the other valves of the plurality of valves configured to operate on the fluid circuit (14) that are configurable to let fluid enter into or exit from the recirculation priming path (Z3) being closed, optionally all the other valves of the plurality of valves configured to operate on the fluid circuit (14) different from the primary concentrate container inlet valve (VGI) and the first concentrate mixing valve (VGM) being closed.

In a 105^{th} aspect according to any one of the previous two aspects, during the recirculation path priming step the control unit (24) is configured to operate the pump (8) at a pre-set pumping rate up to a pre-fixed pumping volume and to stop the pump (8) after said pre-fixed pumping volume has been reached. The pre-fixed pumping volume to pump is lower than the main fluid line volume between fourth junction point (j4) and sixth junction point (j6), but also higher than the volume in the rest of the recirculation priming path (Z3) (i.e., total volume of the recirculation priming path minus the main fluid line volume between fourth junction point (j4) and sixth junction point (j6). In other terms, if for example the volume between the fourth junction point (j4) and the sixth junction point (j6) is 80 ml and the total volume of the recirculation priming path (Z3) is 140 ml, the control unit will be configured to pump 60-80 ml (total volume Z3—volume j4 to j6: 140−80=60 ml). If the control unit (24) were configured to pump less than 60 ml, there would be some air left in the rest of the recirculation priming path (Z3). If the control unit (24) were configured to pump more than 80 ml, the control unit (24) would start refilling the rest of the recirculation priming path (Z3) with air.

In a 106^{th} aspect according to any one of the previous ten aspects, the control unit (24) is configured to execute the first dissolution liquid fill step by controlling the plurality of valves to set a first dissolution liquid fill path (Z4) and by controlling the pump (8) to fill a fraction of an expected total dissolution liquid volume in the primary concentrate container (2), particularly after the air evacuation step and/or after the priming step of the main fluid line (4) and/or after the recirculation path priming step.

In a 107^{th} aspect according to any one of the previous eleven aspects, the control unit (24) is configured to:
control the valves of the plurality of valves to set a first dissolution liquid fill path (Z4) to allow a dissolution liquid to enter into the primary concentrate container (2), the first dissolution liquid fill path (Z4) comprising the at least one portion of the main fluid line (4) on which the pump (8) is configured to operate, a primary concentrate container inlet line (31), the first port (6) of the primary concentrate container (2), the primary concentrate container (2), the second port (7) of the primary concentrate container (2), a portion of a primary concentrate container outlet line (32), optionally a de-aeration chamber (33), and a vent (34), optionally a water inlet valve (VWI) placed on the main fluid line (4), a primary concentrate container inlet valve (VGI) on the primary concentrate container inlet line (31), and a gas valve (VGA) on the vent (34) being open, in particular the other valves of the plurality of valves configured to operate on the fluid circuit (14) that are configurable to let fluid enter into or exit from the first dissolution liquid fill path (Z4) being closed;
run a first dissolution liquid fill step driving the pump (8) to fill a fraction of an expected total dissolution liquid volume in the primary concentrate container (2) through the first dissolution liquid fill path (Z4),
optionally determine and memorize a base pressure (P2_base) in the first dissolution liquid fill path (Z4) to which a pressure controlled filling pressure is compared in subsequent step.

In a 108^{th} aspect according to the previous aspect, in the first dissolution liquid fill path (Z4) the plurality of valves are configured to allow a dissolution liquid to enter into the primary concentrate container (2), the first dissolution liquid fill path comprising the main fluid line (4), a primary concentrate container inlet line (31), the first port (6) of the primary concentrate container (2), the primary concentrate container (2), the second port (7) of the primary concentrate container (2), a portion of a primary concentrate container outlet line (32), a de-aeration chamber (33), and a vent (34) of the de-aeration chamber, optionally a water inlet valve (VWI) connected to the main fluid line (4), a primary concentrate container inlet valve (VGI) on the primary concentrate container inlet line (31), and a gas valve (VGA) on the vent (34) being open, in particular the other valves of the plurality of valves configured to operate on the fluid circuit (14) that are configurable to let fluid enter into or exit from the first dissolution liquid fill path (Z4) being closed, optionally all the other valves of the plurality of valves configured to operate on the fluid circuit (14) different from the water inlet valve (VWI), the primary concentrate container inlet valve (VGI), and the gas valve (VGA) being closed.

In a 109^{th} aspect according to any one of the previous two aspects, during the first dissolution liquid fill step, the control unit (24) is configured to:
operate the pump (8) at a pre-set pumping rate, and
end the first dissolution liquid fill step after a liquid level in a de-aeration chamber (33) reaches a pre-set liquid high level.

In a 110^{th} aspect according to any one of the previous three aspects, during the first dissolution liquid fill step, the control unit (24) is configured to receive pressure data of a pressure in the first dissolution liquid fill path (Z4) and to determine a reference pressure value (P2_base) indicative of the average pressure during at least a part of the first dissolution liquid fill step. For example, pressure may be read through pressure sensor (P2) on the mixing container inlet line experiencing the same pressure existing in the first dissolution liquid fill path (Z4). The reference pressure value (P2_base) may act as a reference pressure for further filling of the concentrate container.

In a 111^{th} aspect according to any one of the previous four aspects, during the first dissolution liquid fill step the control unit (24) operates a heater (23) on the main fluid line (4) to heat the dissolution liquid up to a temperature sufficient to achieve complete dissolution in a certain time period, such as, for a glucose concentrate, at least 70 °C and optionally up to at least 80 °C.

In a 112^{th} aspect according to any one of the previous five aspects, during the first dissolution liquid fill step the control unit (24) is configured to receive pressure data of a pressure in the first dissolution liquid fill path (Z4), said pressure data being stored in a memory associated with the control unit (24), in particular an reference pressure value (P2_base) being stored in the memory.

In a 113^{th} aspect according to any one of the previous sixteen aspects, the control unit (24) is configured to execute the second dissolution liquid fill step by controlling the plurality of valves to set a second dissolution liquid fill path (Z5) and by controlling the pump (8) to fill up to an expected total dissolution liquid volume in the primary concentrate container (2), particularly after the air evacuation step and/or after the priming step of the main fluid line (4) and/or after the recirculation path priming step and/or after the first dissolution liquid fill step.

In a 114^{th} aspect according to any one of the previous seventeen aspects, the control unit (24) is further configured to:
run a second dissolution liquid fill step driving the pump (8) to fill additional dissolution liquid in the primary concentrate container (2) through the second dissolution liquid fill path (Z5),
check a liquid level in a de-aeration chamber (33);
interrupt second dissolution liquid fill step upon reaching a predefined upper level in the de-aeration chamber (33).

In a 115^{th} aspect according to the previous aspect, in the second dissolution liquid fill path (Z5) the plurality of valves are configured to allow dissolution liquid to enter into the primary concentrate container (2), the second dissolution liquid fill path (Z5) comprising the main fluid line (4), a primary concentrate container inlet line (31), the first port (6) of the primary concentrate container (2), the primary concentrate container (2), the second port (7) of the primary concentrate container (2), a portion of a primary concentrate container outlet line (32), a de-aeration chamber (33), and a vent (34) of the de-aeration chamber (33), optionally a water inlet valve (VWI) placed on the main fluid line (4) and a primary concentrate container inlet valve (VGI) on the primary concentrate container inlet line (31) being open, in particular the other valves of the plurality of valves configured to operate on the fluid circuit (14) that are configurable to let fluid enter into or exit from the second dissolution liquid fill path (Z5) being closed, including a gas valve (VGA) on the vent line (34) of the de-aeration chamber (33), optionally all the other valves of the plurality of valves configured to operate on the fluid circuit (14) different from the water inlet valve (VWI) and the primary concentrate container inlet valve (VGI) being closed.

In a 116^{th} aspect according to any one of the previous two aspects, during the second dissolution liquid fill step the control unit (24) is configured to operate the pump (8) at a pre-set pumping rate up to a pre-fixed pressure in the second dissolution liquid fill path (Z5) and to stop the pump (8) after said pre-fixed pressure has been reached and said pre-fixed pressure being a pressure, in particular a reference pressure value (P2_base), in the second dissolution liquid fill path (Z5) plus an overpressure value or an absolute/certain pressure.

In a 116^{th} bis aspect according to the previous aspect and to aspect 110, said pre-fixed pressure is the reference pressure value (P2_base) determined during the first dissolution liquid fill step.

In a 117^{th} aspect according to any one of the previous three aspects, during the second dissolution liquid fill step the control unit (24) is configured to operate a heater (23) on the main fluid line (4) to heat the dissolution liquid up to a temperature sufficient to achieve complete dissolution in a certain time period, such as, for a glucose concentrate, at least 70 °C and optionally up to at least 80 °C.

In a 118^{th} aspect according to any one of the previous five aspects, wherein the control unit (24) is further configured to:
run a final dissolution liquid fill step controlling the pump (8) to fill additional dissolution liquid in the primary concentrate container (2) through the second dissolution liquid fill path (Z5),
measuring pressure in the second dissolution liquid fill path (Z5),
interrupt second dissolution liquid fill step upon reaching the pressure controlled filling pressure, in particular wherein said pressure controlled filling pressure corresponds to base pressure (P2_base) plus a delta, such as 40 mmHg.

In a 119^{th} aspect according to any one of the previous twenty-three aspects, wherein the control unit (24) is configured to execute the concentrate mixture recirculation step by controlling the plurality of valves to set the concentrate recirculation path (Z6) and by controlling the pump (8) to re-circulate the concentrate mixture to dissolve the dry concentrate and homogenously mix the concentrate mixture, the concentrate mixture recirculation step being particularly performed after the air evacuation step and/or after the priming step of the main fluid line (4) and/or after the recirculation path priming step and/or after the first dissolution liquid fill step and/or after the second dissolution liquid fill step.

In a 120^{th} aspect according to any one of the previous twenty-four aspects, wherein the control unit (24) is further configured to:
control the valves of the plurality of valves to set the concentrate recirculation path (Z6);
run a concentrate mixture recirculation step (CMRS) controlling the pump (8) to re-circulate the concentrate liquid mixture to dissolve the dry concentrate and homogenously mix the concentrate liquid mixture, wherein optionally during the concentrate mixture recirculation step, the control unit (24) is configured to operate a heater (23) on the main fluid line (4) to maintain the concentrate liquid mixture flowing through the concentrate recirculation path (Z6) to a temperature in the range between 40 °C and 75 °C.

In a 121^{st} aspect according to the previous aspect, during the concentrate mixture recirculation step the control unit (24) is configured to operate the pump (8) at a pre-set pumping rate for a pre-fixed time period and to stop the pump (8) after said pre-fixed time period has lapsed. The combination of time and flow rate (and possibly also temperature) should be chosen to effectively dissolve the powder and homogenize the solution.

In a 122^{nd} aspect according to any one of the previous two aspects, during the concentrate mixture recirculation step the control unit (24) operates a heater (23) on the main fluid line (4) to heat the concentrate mixture up to a temperature sufficient to achieve complete dissolution in a certain time period, such as, for a glucose concentrate, at least 50 °C and optionally up to at least 60 °C. Again, the combination of time, flow rate, and temperature should be chosen to effectively dissolve the powder and homogenize the solution.

In a 123^{rd} aspect according to any one of the previous twenty-two aspects, the control unit (24) is configured to perform the concentration test step to measure a concentration of the concentrate mixture and to compare the measured concentration with an expected concentration of the concentrate mixture, optionally the control unit (24) being configured to measure said concentration during the concentrate mixture recirculation step.

In a 124^{th} aspect according to any one of the previous aspects, said fluid paths further comprise an air evacuation flow path (Z1), in particular the pump (8) moving the fluid in the air evacuation flow path (Z1).

In a 125^{th} aspect according to any one of the previous aspects, said fluid paths further comprise a main fluid line prime path (Z2), in particular the pump (8) moving the fluid in the main fluid line prime path (Z2).

In a 126^{th} aspect according to any one of the previous aspects, said fluid paths further comprise a recirculation priming path (Z3), in particular the pump (8) moving the fluid in the recirculation priming path (Z3).

In a 127^{th} aspect according to any one of the previous aspects, said fluid paths further comprise a first dissolution liquid fill path (Z4), in particular the pump (8) moving the fluid in the first dissolution liquid fill path (Z4).

In a 128^{th} aspect according to any one of the previous aspects, said fluid paths further comprise a second dissolution liquid fill path (Z5), in particular the pump (8) moving the fluid in the second dissolution liquid fill path (Z5).

In a 129^{th} aspect according to any one of the previous aspects, the control unit (24) is configured to run a liquid concentrate mixing procedure (LCMP), wherein the liquid concentrate mixing procedure (LCMP) includes sending a dilution liquid (DL), e.g., water, to the mixing container (11) and liquid concentrate (LC′; LC″) from at least one of the primary concentrate container (2) and the auxiliary concentrate container (3) to the mixing container (11), to dilute the liquid concentrate (LC′; LC″) and form a diluted liquid mixture.

In a 130^{th} aspect according to the previous aspect, during the liquid concentrate mixing procedure the control unit (24) is configured to heat the dilution liquid (DL) and/or the diluted liquid mixture (DM) by means of a heater (23) optionally placed on the main fluid line (4), wherein in particular the dilution liquid (DL) is heated up to at least 30 °C and more specifically up to at least 37 °C and the diluted liquid mixture (DM) are heated up to at least 30 °C and more specifically up to at least 37 °C.

In a 131^{st} aspect according to any one of the previous two aspects, during the liquid concentrate mixing procedure (LCMP) the control unit (24) is configured to run a diluted mixture recirculation step (RS) in which the control unit (24) is configured to set the mixing recirculation path (R1) and to recirculate the diluted liquid mixture (DM) to homogenize.

In a 132^{nd} aspect according to any one of the previous aspects, the apparatus further comprises a sensor (52) connected to the main fluid line (4) to measure a property of the fluid flowing in the main fluid line (4); the control unit (24) being configured to
receive signals from the sensor (52) related to said property of the fluid flowing in the main fluid line (4);
recirculate the fluid in the mixing recirculation path (R1);
detect a time-resolved variation of said property of the fluid recirculating in the mixing recirculating path (R1), said time-resolved variation of said property of the fluid being related to homogeneity of the fluid recirculating in the mixing recirculating path (R1);
interrupt the recirculation step upon the time-resolved variation meeting a variation criterion.

In a 133^{rd} aspect according to the previous aspect, said property of the fluid flowing in the main fluid line (4) is chosen in the group consisting of a conductivity of the fluid flowing in the main fluid line (4), a concentration of a substance in the fluid flowing in the main fluid line (4), a concentration dependent physical properties, such as sound velocity, viscosity, density, a temperature of the fluid flowing in the main fluid line (4) and combinations thereof, and an optical property of the fluid flowing in the main fluid line (4), in particular said property being either the conductivity or the concentration.

For example a technology based on sound velocity/ultrasound allows to measure concentration. Viscosity may be indirectly measured/correlated from pressure drop. Density may be measured for instance if a volumetric pump is used and the mass flow is measured.

In a 134^{th} aspect according to the previous aspect, the sensor (52) comprises at least one of a conductivity sensor, a concentration sensor, a temperature sensor, an optical sensor, and a sound sensor, in particular said sensor being either a conductivity sensor or a concentration sensor.

In a 135^{th} aspect according to the previous two aspects, to detect the time-resolved variation of the property of the fluid, the control unit (24) is configured to calculate a measure of the dispersion of the signal from the sensor, such as a variance of the signal from the sensor, a standard deviation of the signal from the sensor, a range of the signal from the sensor, an interquartile range of the signal from the sensor, a mean absolute difference of the signal from the sensor, a median absolute deviation of the signal from the sensor, an average deviation of the signal from the sensor, a trend over time of the signal from the sensor, a derivative over time of the signal from the sensor, wherein in particular the time-resolved variation of the property of the fluid comprises the variance of the signal from the sensor.

In a 136^{th} aspect according to the previous three aspects, the time-resolved variation meeting a variation criterion comprises comparing said time-resolved variation of the property of the fluid with a threshold, in particular the variation criterion being met when the time-resolved variation is below said threshold.

In a 137^{th} aspect according to the previous four aspects, the control unit (24) is configured to check whether the time-resolved variation meets the variation criterion after a prefixed period of time has lapsed from the start of the fluid recirculation in the mixing recirculation path (R1), in particular said prefixed period of time being not less than 20 seconds.

In a 138^{th} aspect according to the previous five aspects the sensor (52) is a conductivity meter (25) and the property of the fluid is the conductivity, the control unit (24) being configured to:
detect a variance of the conductivity of the fluid recirculating in the mixing recirculating path (R1),
interrupt the recirculation step after a prefixed time period has lapsed and upon the variance of the conductivity of the fluid is below a threshold, such as 0.2 mS/cm, in particular interrupt the recirculation step (i) after a prefixed time period has lapsed and (ii) upon the variance of the conductivity of the fluid is below a threshold for a pre-set time period.

In a 138^{th} bis aspect according to the previous six aspects, the control unit (24) is configured to check the time-resolved variation in a time window, said time window being for example a fixed time windows such as the last 10 seconds.

In a 138th ter aspect according to the previous aspect, the time window is function of a volume of the mixing recirculation path (R1) and/or a flow rate during fluid recirculation in the mixing recirculation path (R1).

In a 138th quater aspect according to the previous aspect, the time window is proportional to a ratio between the volume and the flow rate, for example the time window being:

tw=const*(V/Q)
wherein
tw is the time window;
const is a constant;
V is the volume of the mixing recirculation path (R1); and
Q is the flow rate during fluid recirculation in the mixing recirculation path (R1).

In a 139^{th} aspect according to any one of the previous three aspects, wherein the liquid concentrate mixing procedure (LCMP) includes one or more of:
a mixing container drain step (MCDS);
a mixing container filling step for rinse (MCFS);
a priming step of the main fluid line (PSMF);
a first filling step (FS′) of an auxiliary liquid concentrate (LC′) from the auxiliary concentrate container (3) into the mixing container (11);
a water filling step (FSW);
a first recirculation step (RS′) of a first diluted liquid mixture (DM′);
a further water filling step (FSW)
a second recirculation step (RS′) of the first diluted liquid mixture (DM′) added with water
a second filling step (FS″) of a primary liquid concentrate (LC″) arriving from the primary concentrate container (2), into the mixing container (11) to form a dialysis mixture (DiaM);
a second recirculation step (RS″) of the dialysis mixture (DiaM) to form the dialysis fluid (DF).

In a 140^{th} aspect according to the previous aspect, the control unit (24) is configured to execute the mixing container drain step (MCDS) by controlling the plurality of valves to set a mixing container drain path (Y1) to allow a drain of the mixing container (11) and by controlling the pump (8) to move the fluid from the mixing container (11) through the outlet (13), at least one portion of the main fluid line (4) on which the pump (8) is configured to operate, a drain outlet line (30) and to a drain port (29).

In a 141^{st} aspect according to the previous aspect, the mixing container drain path (Y1) comprises the mixing container (11), the outlet (13), a mixing container outlet line (16) connecting the outlet (13) of the mixing container (11) to a first junction point (j1) on the main fluid line (4), the at least one portion of the main fluid line (4) on which the pump (8) is configured to operate extending between the first junction point (j1) and the sixth junction point (j6), the drain outlet line (30) from the sixth junction point (j6) to a drain port (29) and the drain port (29).

In a 142^{nd} aspect according to the previous aspect, the mixing container drain path (Y1) comprises a mixing container outlet valve (VMO) configured to operate on the mixing container outlet line (16) and a drain valve (VDR) configured to operate on the drain outlet line (30), when the control unit (24) controls the plurality of valves to set the mixing container drain path (Y1), the mixing container outlet valve (VMO) and the drain valve (VDR) are configured to allow fluid to flow from the mixing container (11) to the drain port (29), in particular the other valves of the plurality of valves configured to operate on the fluid circuit (14) that are configurable to let fluid enter into or exit from the mixing container drain path (Y1) being closed, optionally all the other valves of the plurality of valves configured to operate on the fluid circuit (14) different from the mixing container outlet valve (VMO) and the drain valve (VDR) being closed.

In a 143^{rd} aspect according to any one of the previous three aspects, the control unit (24) is configured to operate the plurality of valves to set the mixing container drain path (Y1), in particular wherein the control unit (24) is configured to first set said mixing container drain path (Y1) prior defining a first concentrate recirculation path (Z6) and/or a second concentrate recirculation path (X5).

In a 144^{th} aspect according to any one of the previous four aspects, during the mixing container drain step (MCDS), particularly in case the mixing container (11) is a flexible mixing container, the control unit (24) is configured to operate the pump (8) at a pre-set pumping rate and to stop the pump (8) when a pre-fixed pressure in the mixing container drain path (Y1) has been reached, optionally said pre-fixed pressure being equal to or lower than a value indicating an empty mixing container, such as −100 mmHg.

In a 144^{th} bis aspect according to any one of the previous four aspects, during the mixing container drain step (MCDS), the control unit (24) is configured to operate the pump (8) at a pre-set pumping rate and to stop the pump (8) when a sensor (52), such as a conductivity meter (25), on the main fluid line (4) senses presence of air in the main fluid line (4).

In a 145^{th} aspect according to any one of the previous six aspects, the control unit (24) is configured to execute the mixing container filling step for rinse (MCFS) by controlling the valves of the plurality of valves to set a mixing container filling path (Y2) to rinse at least the mixing container inlet line (15) and the mixing container (11), during the mixing container filling step for rinse (MCFS) the control unit (24) being configured to control the pump (8) to move the fluid arriving from at least one portion of the main fluid line (4) on which the pump (8) is configured to operate to the mixing container inlet line (15) and the mixing container (11), in particular from the inlet point (5) of the main fluid line (4) of the fluid circuit (14) to the mixing container inlet line (15) and the mixing container (11).

In a 146^{th} aspect according to any one of the previous aspects, the mixing container filling path (Y2) comprises at least one portion of the main fluid line (4) of the fluid circuit (14), particularly the whole main fluid line (4) of the fluid circuit (14), on which the pump (8) is configured to operate, the sixth junction point (j6), the mixing container inlet line (15) and the mixing container (11).

In a 147^{th} aspect according to any one of the previous two aspects, the mixing container filling path (Y2) comprises the water inlet valve (VWI) configured to operate on the main fluid line (4) and the mixing container inlet valve (VMI) configured to operate on the mixing inlet line (15), when the control unit (24) controls the plurality of valves to set the mixing container filling path (Y2), at least the water inlet valve (VWI) and the mixing container inlet valve (VMI) being open to let the fluid flow from the fluid main line (4) to the mixing container (11), optionally the other valves of the plurality of valves, but an air valve (22) if present, configured to operate on the fluid circuit (14) that are configurable to let fluid enter into or exit from the mixing container filling path (Y2) being closed, preferably all the other valves of the plurality of valves configured to operate on the fluid circuit (14) different from the water inlet valve (VWI) and the mixing container inlet valve (VMI) being closed. In case the mixing container (11) is a rigid container with an air vent and a corresponding air valve (22), then air valve (22) should also be open to let air to be expelled during filling.

In a 148^{th} aspect according to any one of the previous three aspects, during the mixing container filling step for rinse (MCFS), the control unit (24) is configured to operate the pump (8) at a pre-set pumping rate up to a pre-fixed pumping volume and to stop the pump (8) after said pre-fixed pumping volume has been reached, optionally said pre-fixed pumping volume being sufficient to achieve proper rinse of the main fluid line (4) and the mixing container (11).

In a 149^{th} aspect according to any one of the previous four aspects, the mixing container filling step for rinse (MCFS) comprises a mixing container emptying step (MCES), wherein the control unit (24) is configured to control the valves of the plurality of valves to set a mixing container emptying path to rinse at least the mixing container outlet line (16), the control unit (24) being further configured to control the pump (8) to move the fluid arriving from the mixing container (11) to the drain outlet line (30) and the drain port (29).

In a 150^{th} aspect according to the previous aspect, the mixing container emptying path comprises the mixing container outlet line (16), the first junction point (j1) on the main fluid line (4), at least one portion of the main fluid line (4), the sixth junction point (j6), the drain outlet line (30) and the drain port (29).

In a 151^{st} aspect according to any one of the previous two aspects, the mixing container emptying path comprises the mixing container outlet valve (VMO) configured to operate on the mixing container outlet line (16) and the drain valve (VDR) configured to operate on the drain outlet line, when the control unit (24) controls the plurality of valves to set the mixing container emptying path at least the mixing container outlet valve (VMO) and the drain valve (VDR) being open to let the fluid flow from the mixing container (11) to the drain port (29), optionally the other valves of the plurality of valves configured to operate on the fluid circuit (14) that are configurable to let fluid enter into or exit from the mixing container emptying path being closed, preferably all the other valves of the plurality of valves configured to operate on the fluid circuit (14) different from the water inlet valve (VWI) and the mixing container inlet valve (VMI) being closed.

In a 152^{nd} aspect according to any one of the previous three aspects, the control unit (24) is configured to execute the mixing container emptying step (MCES) after the mixing container filling step for rinse (MCFS).

In a 153^{rd} aspect according to any one of the previous four aspects, during the mixing container emptying step (MCES), particularly in case the mixing container (11) is a flexible mixing container, the control unit (24) is configured to operate the pump (8) at a pre-set pumping rate and to stop the pump (8) when a pre-fixed pressure in the mixing container emptying path has been reached.

In a 153^{rd} bis aspect according to any one of the previous five aspects, during the mixing container emptying step (MCES), the control unit (24) is configured to operate the pump (8) at a pre-set pumping rate and to stop the pump (8) when a sensor (52), such as a conductivity meter (25), on the main fluid line (4) senses presence of air in the main fluid line (4).

In a 154^{th} aspect according to any one of the previous fifteen aspects, the control unit (24) is configured to execute the priming step of the main fluid line (PSMF) by controlling the valves of the plurality of valves to set a main fluid priming path to rinse the main fluid line (4), and by controlling the pump (8) to move the fluid arriving from the inlet point (5) at least to the drain outlet line (30).

In a 155^{th} aspect according to the previous aspect, the main fluid priming path comprises at least one portion of the main fluid line (4), particularly the whole main fluid line (4) of the fluid circuit (14), on which the pump (8) is configured to operate, the main fluid priming path developing between the inlet point (5) and the outlet drain line (30), specifically between the inlet point (5) and the drain port (29).

In a 156^{th} aspect according to any one of the previous two aspects, the main fluid priming path comprises the water inlet valve (VWI) configured to operate on the main fluid line (4) and the drain valve (VDR) configured to operate on the outlet drain line (30), when the control unit (24) controls the plurality of valves to set the main fluid priming path, at least the water inlet valve (VWI) and the drain valve (VDR) being open to let the fluid flow from the fluid main line (4) to the drain outlet line (30), optionally the other valves of the plurality of valves configured to operate on the fluid circuit (14) that are configurable to let fluid enter into or exit from the main fluid priming path being closed, preferably all the other valves of the plurality of valves configured to operate on the fluid circuit (14) different from the water inlet valve (VWI) and the drain valve (VDR) being closed.

In a 157^{th} aspect according to any one of the previous three aspects, during the priming step of the main fluid line (PSMF), the control unit (24) is configured to operate the pump (8) at a pre-set pumping rate and until a pre-fixed time period has lapsed. The pump (8) is run at least until after a volume contained between first junction point (j1) and sixth junction point (j6) has been pumped, in particular with the aim that any residual conductivity is low enough to not significantly impact the composition accuracy in the following steps.

In a 158^{th} aspect according to the previous aspect, during the priming step of the main fluid line (PSMF), the control unit (24) operates the heater (23) on the main fluid line (4) to heat the fluid flowing through the main fluid priming path up to 37 °C.

In a 159^{th} aspect according to any one of the previous two aspects, the control unit (24) is configured to perform a main fluid line concentration test step (MF-CTS) to measure a concentration value of the fluid flowing through the main fluid priming path, and to compare the concentration value measured in the fluid flowing through the main fluid priming path with a concentration reference value, particularly not exceeding 0.2 mS/cm, preferably not exceeding 0.15 mS/cm, optionally the control unit being configured to end the priming step of the main fluid line when the conductivity is below a pre-fixed threshold, such as 0.2 mS/cm.

In a 160^{th} aspect according to any one of the previous six aspects, the control unit (24) is configured to execute the priming step of the main fluid line (PSMF) after the mixing container filling step for rinse (MCFS) and/or the mixing container emptying step (MCES).

In a 161^{st} aspect according to any one of the previous aspects, the control unit (24) is configured to execute a first filling step (FS′) of an auxiliary liquid concentrate (LC′) by controlling the valves of the plurality of valves to set an auxiliary concentrate filling path (F1) to provide the auxiliary liquid concentrate (LC′) to the mixing container (11), particularly according to a pre-fixed nominal volume, during the first filling step (FS′) of the auxiliary liquid concentrate (LC′) the control unit (24) being configured to operate the pump (8) to move the auxiliary liquid concentrate (LC′) from the corresponding concentrate container 2, 3, particularly from the auxiliary concentrate container (3), to the mixing container (11).

In a 162^{nd} aspect according to the previous aspect, the auxiliary concentrate filling path (F1) comprises a second concentrate mixing line (41), the second junction point (j2), at least one portion of the main fluid line (4) in which the pump (8) is configured to operate, the sixth junction point (j6), the mixing container mixing line (15), the inlet (12) of the mixing container (11) and the mixing container (11).

In a 163^{rd} aspect according to any one of the previous two aspects, the auxiliary concentrate filling path (F1) comprises the second concentrate mixing valve (VAM) configured to operate on the second concentrate mixing line (41) and the mixing container inlet valve (VMI) configured to operate on the mixing inlet line (15), when the control unit (24) controls the plurality of valves to set the auxiliary concentrate filling path (F1), the second concentrate mixing valve (VAM) and the mixing container inlet valve (VMI) being open to let the auxiliary liquid concentrate (LC′) of the auxiliary concentrate container (3) to move to the mixing container (11), optionally the other valves of the plurality of valves configured to operate on the fluid circuit (14) that are configurable to let fluid enter into or exit from the auxiliary concentrate filling path (F1) being closed, preferably all the other valves of the plurality of valves configured to operate on the fluid circuit (14) different from the mixing container inlet valve (VMI) and the mixing container inlet valve (VMI) being closed.

In a 164^{th} aspect according to the previous aspect 161, the auxiliary concentrate filling path (F1) comprises the auxiliary concentrate container outlet line (40), the third junction point (j3), at least one portion of the main fluid line (4) in which the pump (8) is configured to operate, the sixth junction point (j6), the mixing container mixing line (15), the inlet (12) of the mixing container (11) and the mixing container (11).

In a 165^{th} aspect according to any one of the previous aspect, the auxiliary concentrate filling path (F1) comprises the auxiliary concentrate container outlet valve (VAO) configured to operate on the auxiliary concentrate container outlet (40) and the mixing container inlet valve (VMI) configured to operate on the mixing inlet line (15), when the control unit (24) controls the plurality of valves to set the auxiliary concentrate filling path (F1), the auxiliary concentrate container outlet valve (VAO) and the mixing container inlet valve (VMI) being open to let the auxiliary concentrate of the auxiliary concentrate container (3) to move to the mixing container (11), optionally the other valves of the plurality of valves configured to operate on the fluid circuit (14) that are configurable to let fluid enter into or exit from the auxiliary concentrate filling path (F1) being closed, preferably all the other valves of the plurality of valves configured to operate on the fluid circuit (14) different from the auxiliary concentrate container outlet valve (VAO) and the mixing container inlet valve (VMI) being closed.

In 166^{th} aspect according to any one of the previous five aspects, during the first filling step (FS′) of the auxiliary liquid concentrate (LC′), the control unit (24) being configured to operate the pump (8) at a pre-set pumping rate up to a pre-fixed pumping volume and to stop the pump (8) after said pre-fixed pumping volume has been reached. Pre-fixed pumping volume is selected to fill the mixing container (11) with a nominal volume of the auxiliary liquid concentrate (LC′) according to a medical fluid composition to be prepared.

In a 167^{th} aspect according to any one of the previous six aspects, during the first filling step (FS′) of the auxiliary liquid concentrate (LC′), the control unit (24) is configured to operate the heater (23) on the main fluid line (4) to heat the auxiliary liquid concentrate (LC′) flowing through the auxiliary concentrate filling path (F1) up to e.g., 37 °C.

In a 168^{th} aspect according to any one of the previous seven aspects, the control unit (24) is configured to execute the first filling step (FS′) of the auxiliary liquid concentrate (LC′) after the mixing container drain step MCDS and/or after the mixing container filling step for rinse (MCFS) and/or after the mixing container emptying step (MCES) and/or after the priming step of the main fluid line (PSMF).

In a 169^{th} aspect according to any one of the previous six aspects, the filling step (FS′) of the auxiliary liquid concentrate (LC′) is followed by at least one filling step of water (FSW), wherein the control unit (24) is configured to control the valves of the plurality of valves to set a water filling path (W1) to fill the mixing container (11), at least partially, with a pre-fixed volume of water, the control unit (24) being configured to control the pump (8) to flow the water arriving from the inlet point (5) of the fluid circuit (14) to the mixing container (11).

In a 170^{th} aspect according to the previous aspect, the water filling path (FSW) comprises at least one portion of the main fluid line (4), particularly developing between the inlet point (5) of the fluid circuit (14) and the sixth junction point (j6), the sixth junction point (j6), the mixing container inlet line (15), the inlet (12) of the mixing container (11) and the mixing container (11).

In a 171^{st} aspect according to any one of the previous two aspects, the water filling path (W1) comprises the water inlet valve (VWI) configured to operate on the fluid main line (4) and the mixing container inlet valve (VMI) configured to operate on the mixing inlet line (15), when the control unit (24) controls the plurality of valves to set the water filling path (W1), the water inlet valve (VWI) and the mixing container inlet valve (VMI) being open to let the water arriving from the inlet point (5) of the fluid circuit (14) to flow to the mixing container (11), optionally the other valves of the plurality of valves configured to operate on the fluid circuit (14) that are configurable to let fluid enter into or exit from the water filling path (W1) being closed, preferably all the other valves of the plurality of valves configured to operate on the fluid circuit (14) different from the water inlet valve (VWI) and the mixing container inlet valve (VMI) being closed.

In a 172^{nd} aspect according to any one of the previous three aspects, during the filling step of water (FSW), the control unit (24) is configured to operate the pump (8) at a pre-set pumping rate up to a pre-fixed pumping volume and to stop the pump (8) after said pre-fixed pumping volume has been reached.

In a 173^{rd} aspect according to the previous aspect, the control unit (24) is configured to determine a water dead volume and to correct the pre-fixed pumping volume to be at least 80% of a calculated reference volume of water minus the water dead volume.

In a 174^{th} aspect according to the previous aspects 172 or 173, the calculated reference volume of water is obtained by a pre-fixed nominal volume of water minus a dead volume of water, particularly defined by a volume of water remaining into the fluid circuit (14), optionally into the mixing container drain path Y1 and/or the mixing container filling path Y2 and/or the mixing container emptying path and/or the main fluid priming path.

In a 175^{th} aspect according to any one of the previous three aspects, during the filling step of water, the control unit (24) is configured to operate the heater (23) on the main fluid line (4) to heat the water flowing through the filling path of water up to a temperature suitable for conductivity sensing e.g., 37 °C. Since temperature influences the conductivity sensor reading, the heater raises the water temperature to a value allowing to achieve a precise conductivity measurement.

In a 176^{th} aspect according to any one of the previous seven aspects, in the first recirculation step (RS′) of the first diluted liquid mixture (DM′) added with water, the control unit (24) is configured to operate the valves of the plurality of valves to set a mixing recirculation path (R1) to mix the first diluted liquid mixture (DM′) added with water filled into the mixing container (11), during the first recirculation filling step (RS′) of the first diluted liquid mixture (DM′) added with water, the control unit (24) being configured to operate the pump (8) to flow these latter through the mixing recirculation path (R1).

In a 177^{th} aspect according to the previous aspect, the mixing recirculation path (R1) comprises the mixing container (11), the outlet (13) of the mixing container (11), the mixing container outlet line (16) and the first junction point (j1), at least one portion of the main fluid line (4) in which the pump (8) is configured to operate, particularly developing between the first junction point (j1) and the sixth junction point (j6), the sixth junction point (j6) and the mixing container inlet line (15).

In a 178^{th} aspect according to any one of the previous two aspects, the mixing recirculation path (R1) comprises the mixing container outlet valve (VMO) and the mixing container inlet valve (VMI), wherein the control unit (24) is configured to control the plurality of valves to set the mixing recirculation path (R1), the mixing container outlet valve (VMO) and the mixing container inlet valve (VMI) being open to let the first diluted liquid mixture (DM′) to flow through the mixing recirculation path (R1), optionally the other valves of the plurality of valves configured to operate on the fluid circuit (14) that are configurable to let fluid enter into or exit from the mixing recirculation path being closed, preferably all the other valves of the plurality of valves configured to operate on the fluid circuit (14) different from the mixing container outlet valve (VMO) and the mixing container inlet valve (VMI) being closed.

In a 179^{th} aspect according to any one of the previous three aspects, during the first recirculation step (RS′) of the first diluted liquid mixture (DM′), the control unit (24) is configured to operate the pump (8) at a pre-set pumping rate and at least for a pre-fixed time period has lapsed. In particular, pump (8) is run until a volume equal to at least the volume contained between the mixing container outlet (13) and sixth junction point (j6) has been pumped. A further requirement before stopping the pump 8 may be the one of subsequent aspect 181.

In a 180^{th} aspect according to the previous aspect, during the first recirculation step (RS′) of the first diluted liquid mixture (DM′), the control unit (24) is configured to operate the heater (23) on the main fluid line (4) to heat the first diluted liquid mixture (DM′) up to a temperature suitable for conductivity sensing e.g., 37 °C.

In a 181^{st} aspect according to any one of the previous two aspects, the control unit (24) is configured to perform a further main fluid line concentration test step (MF-CTS′) to detect, by means of at least one conductivity meter (25, 26), preferably continuously, conductivity variances of the first diluted liquid mixture (DM′) flowing through the mixing recirculation path (R1), optionally the control unit (24) being configured to compare the conductivity variances detected in the first diluted liquid mixture (DM′) flowing through the mixing recirculation path (R1) with a pre-fixed threshold, under which a signal of interruption of the first recirculation step (RS′) of the first diluted liquid mixture (DM′) is emitted.

In a 182^{nd} aspect according to any one of the previous six aspects, the filling step of water (FSW) and the first recirculation step (RS′) of the first diluted liquid mixture (DM′) are repeated at least once.

In a 183^{rd} aspect according to the previous aspect, during the repeated filling step of water (FSW), the control unit (24) is configured to operate the pump (8) at a pre-set pumping rate up to a pre-fixed pumping volume and to stop the pump (8) after said pre-fixed pumping volume has been reached, optionally said pre-fixed pumping volume being equal to or higher than a recalculated reference volume of water.

In a 184^{th} aspect according to any one of the previous two aspects, during the repeated filling step of water (FSW), the control unit (24) is configured to operate the heater (23) on the main fluid line (4) to heat the water and/or the first diluted liquid mixture (DM′) added with water flowing through the filling path of water and/or the mixing recirculation path (R1) up to a value allowing to achieve a precise conductivity measurement e.g., 37 °C.

In a 185^{th} aspect according to any one of the previous three aspects, during the repeated first recirculation step (R1) of the first diluted liquid mixture (DM′) added with water, the control unit (24) is configured to operate the pump (8) at a pre-set pumping rate and at least for a pre-fixed time period. In particular, pump (8) is run until a volume equal to at least the volume contained between the first junction point (j1) and the sixth junction point (j6) has been pumped. A further requirement before stopping the pump 8 may be the one of subsequent aspect 187.

In a 186^{th} aspect according to the previous aspect, during the repeated first recirculation step (RS′) of the first diluted liquid mixture (DM′) added with water, the control unit (24) is configured to operate the heater (23) on the main fluid line (4) to heat the first diluted liquid mixture (DM′) added with water up to a value allowing to achieve a precise conductivity measurement e.g., 37 °C.

In a 187^{th} aspect according to any one of the previous two aspects, the control unit (24) is configured to perform a further main fluid line concentration test step (MF-CTS′) to detect, by means of at least one conductivity meter (25, 26), preferably continuously, conductivity variances of the first diluted liquid mixture (DM′) added with water flowing through the mixing recirculation path (R1), optionally the control unit (24) being configured to compare the conductivity variances detected in the first diluted liquid mixture (DM′) added with water flowing through the mixing recirculation path (R1) with a pre-fixed threshold, under which a signal of interruption of the repeated first recirculation step (RS′) of the first diluted liquid mixture (DM′) added with water is emitted.

In a 188^{th} aspect according to any one of the previous twelve aspects, in a second filling step (FS″) of the primary liquid concentrate (LC″) to form a dialysis mixture (DiaM), the control unit (24) is configured to control the valves of the plurality of valves to set a primary concentrate filling path (F2) to fill the mixing container (11) with the primary liquid concentrate (LC″), particularly according to a pre-fixed nominal volume, in the filling step of the primary liquid concentrate the control unit (24) being further configured to control the pump (8) to move the primary liquid concentrate (LC″) to the mixing container (11).

In a 189^{th} aspect according to the previous aspect, the primary concentrate filling path (F2) comprises a first concentrate mixing line (36), the fourth junction point (j4), at least one portion of the main fluid line (4) in which the pump (8) is configured to operate, the sixth junction point (j6), the mixing container mixing line (15), the inlet (12) of the mixing container (11) and the mixing container (11).

In a 190^{th} aspect according to any one of the previous two aspects, the primary concentrate filling path (F2) comprises the first concentrate mixing valve (VGM) configured to operate on the first concentrate mixing line (36) and the mixing container inlet valve (VMI) configured to operate on the mixing inlet line (15), when the control unit (24) controls the plurality of valves to set the primary concentrate filling path (F2), the first concentrate mixing valve (VGM) and the mixing container inlet valve (VMI) being open to let the primary liquid concentrate (LC″) arriving from the primary concentrate container (2) to move to the mixing container (11), optionally the other valves of the plurality of valves configured to operate on the fluid circuit (14) that are configurable to let fluid enter into or exit from the primary concentrate filling path (F2) being closed, preferably all the other valves of the plurality of valves configured to operate on the fluid circuit (14) different from the first concentrate mixing valve (VGM) and the mixing container inlet valve (VMI) being closed.

In a 191^{st} aspect according to any one of the previous aspects 188, the primary concentrate filling path (F2) comprises the primary concentrate container outlet line (32), the fifth junction point (j5), at least one portion of the main fluid line (4) in which the pump (8) is configured to operate, the sixth junction point (j6), the mixing container mixing line (15), the inlet (12) of the mixing container (11) and the mixing container (11).

In a 192^{nd} aspect according to the previous aspect, the primary concentrate filling path (F2) comprises the primary concentrate container outlet valve (VGO) configured to operate on the primary concentrate container outlet (32) and the mixing container inlet valve (VMI) configured to operate on the mixing inlet line (15), when the control unit (24) controls the plurality of valves to set the primary concentrate filling path (F2), the primary concentrate container outlet valve (VGO) and the mixing container inlet valve (VMI) being open to let the primary liquid concentrate (LC″) arriving from the primary concentrate container (2) to move to the mixing container (11), optionally the other valves of the plurality of valves configured to operate on the fluid circuit (14) that are configurable to let fluid enter into or exit from the primary concentrate filling path (F2) being closed, preferably all the other valves of the plurality of valves configured to operate on the fluid circuit (14) different from the primary concentrate container outlet valve (VGO) and the mixing container inlet valve (VMI) being closed.

In a 193^{rd} aspect according to any one of the previous five aspects, during the second filling step (FS″) of the primary liquid concentrate (LC″), the control unit (24) is configured to operate the pump (8) at a pre-set pumping rate up to a pre-fixed pumping volume and to stop the pump (8) after said pre-fixed pumping volume has been reached, optionally said pre-fixed pumping volume being equal to or higher than a recalculated reference volume of the primary liquid concentrate (LC″).

In a 194^{th} aspect according to any one of the previous six aspects, during the second filling step (FS″) of the primary liquid concentrate (LC″), the control unit (24) is configured to operate the heater (23) on the main fluid line (4) to heat the primary liquid concentrate (LC″) flowing through the primary concentrate filling path up to a temperature suitable for conductivity sensing e.g., 37 °C.

In a 195^{th} aspect according to any one of the previous seven aspects, the control unit (24) is configured to execute the second filling step (FS″) of the primary liquid concentrate (LC″) after the first recirculation step (RS′) of the first diluted liquid mixture (DM′) added with water and/or after the repetition filling step of water FSW and/or after the repetition of the first recirculation step RS' of the first diluted liquid mixture (DM′).

In a 196^{th} aspect according to any one of the previous eight aspects, the second filling step of the primary liquid concentrate (LC″) is followed by at least one second recirculation step (RS″) of the dialysis mixture (DiaM) formed by adding the primary liquid concentrate (LC″) to the first diluted liquid mixture (DM″) in the mixing container (11), during the second recirculation step (RS″) of the dialysis mixture (DiaM), the control unit (24) is configured to control the valves of the plurality of valves to set a mixing recirculation path allowing the dialysis mixture (DiaM) to mix and to form the dialysis fluid (FD), during the second recirculation step (RS″) of the dialysis mixture (DiaM) the control unit (24) being further configured to operate the pump (8) to flow the dialysis mixture (DiaM) through the mixing recirculation path.

In a 197^{th} aspect according to the previous aspect, the mixing recirculation path comprises the mixing container (11), the outlet (13) of the mixing container (11), the mixing container outlet line (16) and the first junction point (j1), at least one portion of the main fluid line (4) in which the pump (8) is configured to operate, particularly developing between the first junction point (j1) and the sixth junction point (j6), the sixth junction point (j6), the mixing container inlet line (15) and the inlet (12).

In a 198^{th} aspect according to any one of the previous two aspects, the mixing recirculation path comprises the mixing container outlet valve (VMO) configured to operate on the mixing outlet line (16) and the mixing container inlet valve (VMI) configured to operate on the mixing inlet line (15), when the control unit (24) controls the plurality of valves to set the mixing recirculation path, the mixing container outlet valve (VMO) and the mixing container inlet valve (VMI) being open to let the dialysis fluid to flow through the mixing recirculation path, optionally the other valves of the plurality of valves configured to operate on the fluid circuit (14) that are configurable to let fluid enter into or exit from the mixing recirculation path being closed, preferably all the other valves of the plurality of valves configured to operate on the fluid circuit (14) different from the mixing container outlet valve (VMO) and the mixing container inlet valve (VMI) being closed.

In a 199^{th} aspect according to any one of the previous three aspects, during the second recirculation step (RS″) of the dialysis mixture (DiaM), the control unit (24) is configured to operate the pump (8) at a pre-set pumping rate and at least for a pre-fixed time period. In particular, pump (8) is run until a volume equal to at least the volume contained between the first junction point (j1) and the sixth junction point (j6) has been pumped. A further requirement before stopping the pump 8 may be the one of subsequent aspect 201.

In a 200^{th} aspect according to the previous aspect, during the recirculation step (RS″) of the dialysis mixture (DiaM), the control unit (24) is configured to operate the heater (23) on the main fluid line (4) to heat the dialysis mixture (DiaM) up to a temperature suitable for conductivity sensing e.g., 37 °C.

In a 201^{st} aspect according to any one of the previous two aspects, the control unit (24) is configured to perform an additional main fluid line concentration test step (MF-CTS″) to detect, by means of at least one conductivity meter (25, 26), preferably continuously, conductivity variances of the dialysis mixture (DiaM) flowing through the mixing recirculation path, optionally the control unit (24) being configured to compare the conductivity variances detected in the dialysis mixture (DiaM) with a pre-fixed threshold, under which a signal of interruption of the second recirculation step (RS″) of the dialysis mixture (DiaM) is emitted.

In a 202^{nd} aspect according to any one of the previous aspects, an auxiliary concentrate container (3) in fluid communication with the first auxiliary container access (45, 46) houses an auxiliary liquid concentrate (LC′), wherein the control unit (24) is configured to:
control the valves of the plurality of valves to set an auxiliary concentrate filling path (F1) to allow filling the mixing container (11) with the auxiliary liquid concentrate (LC′);
run a first filling step (FS′) of the auxiliary liquid concentrate (LC′) to fill the mixing container (11), particularly according to a pre-fixed nominal volume, with the auxiliary liquid concentrate (LC′);
control the valves of the plurality of valves to set a water filling path (W1) to allow filling the mixing container (11) with water;
run a filling step of water (FSW) to fill the mixing container (11) with water, particularly according to a pre-fixed nominal water volume, wherein optionally during the filling step of water, the control unit (24) is configured to operate a heater (23) on the main fluid line (4) to maintain the water flowing through the water filling path (W1) to a temperature in the range between 30 and 45 °C.

In a 203^{rd} aspect according to the previous aspect, prior running the first filling step (FS′), the control unit (24) is configured to:
control the valves of the plurality of valves to set a mixing container drain path (Y1) to allow emptying the mixing container (11);
run a mixing container emptying step (MCES) to empty the mixing container (11) from fluid contained therein;
control the valves of the plurality of valves to set a main fluid line prime path (Z2) to allow rinsing the main fluid line (4);
run a priming step of the main fluid line (MFPS) to prime the main fluid line (4);
interrupt the priming step of the main fluid line (MFPS) after a prefixed time period has lapsed and/or after the property of the fluid flowing in the main fluid line (4) measured with a sensor (52) matches a priming criteria.

In a 204^{th} aspect according to the previous aspect, during the priming step of the main fluid line (MFPS), water remains in the main fluid line (4), wherein, when running said filling step of water (FSW), the control unit (24) is configured to control pump (8) to fill the mixing container (11) with said pre-fixed nominal water volume and to determine said pre-fixed nominal water volume taking into account an amount of water already contained in the main fluid line (4).

In a 205^{th} aspect according to any one of the previous three aspects, the control unit (24) is configured to run a first recirculation step (RS′) comprising to:
operate the plurality of valves to configure the mixing recirculation path (R1);
operate the pump (8) to recirculate in the mixing recirculation path (R1) a recirculating fluid comprising the auxiliary liquid concentrate (LC′) and the water filled into mixing container (11);
wherein the control unit (24) further is configured to perform a fluid homogeneous mixing test step (MF-CTS′) comprising said detection of the time-resolved variation of said property of the fluid recirculating in the mixing recirculating path (R1) and said interruption of the first recirculation step.

In a 206^{th} aspect according to any one of the previous four aspects, the control unit (24) is configured to:
measure the property of the fluid flowing in the main fluid line (4) during fluid recirculation in the mixing recirculating path (R1);
control the valves of the plurality of valves to set the water filling path (W1) to allow filling the mixing container (11) with water;
run a further filling step of water to fill the mixing container (11) with additional water, particularly according to a water volume calculated based on said measure of the property of the fluid during fluid recirculation;
optionally control the valves of the plurality of valves to set the mixing recirculating path (R1) and recirculate the previously mixed auxiliary liquid concentrate (LC′) and water with the additional water in a second recirculation step, wherein the control unit (24) is further configured to perform a further fluid homogeneous mixing test step (MF-CTS′) comprising said detection of the time-resolved variation of said property of the fluid recirculating in the mixing recirculating path (R1) and said interruption of the second recirculation step.

In a 207^{th} aspect according to any one of the previous five aspects, a primary concentrate container (2) in fluid communication with the first primary container access (43) houses a primary liquid concentrate (LC″), wherein the control unit (24) is configured to:
control the valves of the plurality of valves to set a primary concentrate filling path (F2) to allow filling the mixing container (11) with the primary liquid concentrate (LC″);
run a second filling step (FS″) of the primary liquid concentrate (LC″) to fill the mixing container (11), particularly according to a calculated volume, with the primary liquid concentrate (LC″), wherein optionally during the second filling step of the primary liquid concentrate (LC″), the control unit (24) is configured to operate a heater (23) on the main fluid line (4) to maintain the water flowing through the primary concentrate filling path (F2) to a temperature in the range between 30 and 45 °C.

In a 208^{th} aspect according to the previous aspect, the control unit (24) is configured to run a final recirculation step (RS′) comprising to:
operate the plurality of valves to configure the mixing recirculation path (R1);
operate the pump (8) to recirculate in the mixing recirculation path (R1) a recirculating fluid comprising the auxiliary liquid concentrate (LC′), the primary liquid concentrate (LC″) and the water filled into mixing container (11) to form a dialysis mixture (DiaM);
   wherein the control unit (24) is further configured to perform a final fluid homogeneous mixing test step (MF-CTS″) comprising said detection of the time-resolved variation of said property of the fluid recirculating in the mixing recirculating path (R1) and said interruption of the final recirculation step.

In a 209^{th} aspect according to any one of the previous aspects, the control unit (24) is configured to run a dry concentrate dissolution procedure, wherein the dry concentrate dissolution procedure includes:
sending a dissolution liquid, e.g., water, to at least one of the primary concentrate container (2) and the auxiliary concentrate container (3), to dissolve a dry concentrate and form a concentrated liquid mixture;
heating the dissolution liquid and/or the concentrated liquid mixture by means of a heater (23) placed on the main fluid line (4), wherein in particular the dissolution liquid is heated up to at least 60 °C and more specifically up to at least 70 °C and the concentrated mixture is heated up to at least 40 °C and more specifically up to at least 50 °C;
a concentrate liquid mixture recirculation step in which the control unit (24) sets the concentrate recirculation path (Z6, X5) and recirculates the concentrated liquid mixture to homogenize.

In a 210^{th} aspect according to the previous aspect, the dry concentrate dissolution procedure includes a dissolution liquid fill step comprising a first dissolution liquid fill step, wherein the first dissolution liquid fill step comprises the control unit (24) driving the plurality of valves to set a first dissolution liquid fill path (Z4) and driving the pump (8) to fill a fraction of an expected total dissolution liquid volume in the primary concentrate container (2), the plurality of valves being configured to allow a dissolution liquid to enter into the primary concentrate container (2), the first dissolution liquid fill path (Z4) comprising the at least one portion of the main fluid line (4) on which the pump (8) is configured to operate, a primary concentrate container inlet line (31), the first port (6) of the primary concentrate container (2), the primary concentrate container (2), the second port (7) of the primary concentrate container (2), a portion of a primary concentrate container outlet line (32), optionally a de-aeration chamber (33), and a vent (34), optionally a water inlet valve (VWI) placed on the main fluid line (4), a primary concentrate container inlet valve (VGI) on the primary concentrate container inlet line (31), and a gas valve (VGA) on the vent (34) being open, in particular the other valves of the plurality of valves configured to operate on the fluid circuit (14) that are configurable to let fluid enter into or exit from the first dissolution liquid fill path (Z4) being closed, optionally all the other valves of the plurality of valves configured to operate on the fluid circuit (14) different from the water inlet valve (VWI), the primary concentrate container inlet valve (VGI), and the gas valve (VGA) being closed.

In a 211^{th} aspect according to any one of the previous two aspects, during the concentrate liquid mixture recirculation step the control unit (24) is configured to control the plurality of valves to set the concentrate recirculation path (Z6) and to control the pump (8) to re-circulate the concentrate liquid mixture to dissolve the dry concentrate and homogenously mix the concentrate liquid mixture, wherein the control unit (24) is configured to perform a concentration test step to measure a concentration of the concentrate mixture and to compare the measured concentration with an expected concentration of the concentrate mixture, the control unit (24) being configured to measure said concentration during the concentrate mixture recirculation step.

In a 212^{th} aspect according to any one of the previous aspects, at least one of:
a primary concentrate container (2) in fluid communication with the first primary container access (43, 44); and
an auxiliary concentrate container (3) in fluid communication with the first auxiliary container access (45, 46);
   houses a diluted liquid mixture (DM′), wherein the diluted liquid mixture (DM′) is a liquid concentrate (LC′; LC″) previously diluted with a dilution liquid (DLL), e.g., water,
   wherein the control unit (24) is configured to run a liquid concentrate mixing procedure including a first filling step (FS′) of first diluted mixture (DM′), the control unit (24) being configured to control the valves of the plurality of valves to set an auxiliary concentrate filling path (F1) to fill the mixing container (11) with the diluted liquid mixture (DM′), particularly according to a pre-fixed nominal volume, during the first filling step (FS′) of first diluted liquid mixture (DM′) the control unit (24) being further configured to control the pump (8) to move the diluted liquid mixture (DM′) from the corresponding primary concentrate container (2), or auxiliary concentrate container (3) to the mixing container (11).

In a 213^{th} aspect according to the previous aspect, the filling step (FS′) of the first diluted mixture (DM′) is followed by at least one filling step of water (FSW) wherein the control unit (24) is configured to control the valves of the plurality of valves to set a water filling path (W1) to fill the mixing container (11), at least partially, with a pre-fixed volume of water, during the filling step of water the control unit (24) being further configured to control the pump (8) to flow the water arriving from the inlet point (5) of the fluid circuit (14) to the mixing container (11), wherein the water filling path (FSW) comprises at least one portion of the main fluid line (4) in which the pump (8) is configured to operate, developing between the inlet point (5) of the fluid circuit (14) and the sixth junction point (j6), the mixing container inlet line (15), the inlet (12) of the mixing container (11) and the mixing container (11), in particular wherein during the filling step of water, the control unit (24) being configured to operate the heater (23) on the main fluid line (4) to heat the water flowing through the filling path of water up to a temperature suitable for (subsequent) conductivity sensing e.g., 37 °C.

In a 214^{th} aspect according to the previous aspect, the control unit (24) is configured to run a first recirculation step (RS′) of the first diluted liquid mixture (DM′), during the first recirculation step (RS′) the control unit (24) being configured to control the valves of the plurality of valves to set a mixing recirculation path (R1) to mix the first diluted liquid mixture (DM′) and the water filled into the mixing container (11), during the first recirculation filling step (RS′) of the first diluted liquid mixture (DM′) added with water the control unit (24) being further configured to control the pump (8) to flow the first diluted liquid mixture (DM′) and the water through the mixing recirculation path (R1), wherein the control unit (24) is configured to perform a main fluid line concentration test step (MF-CTS′) to detect, by means of at least one conductivity meter (25, 26), conductivity variances of the first diluted liquid mixture (DM′) added with water flowing through the mixing recirculation path (R1), the control unit (24) being configured to compare the conductivity variances detected in the first diluted liquid mixture (DM′) added with water flowing through the mixing recirculation path (R1) with a pre-fixed threshold, and to interrupt the first recirculation step (RS′) of the first diluted liquid mixture (DM′) when the conductivity variance is below the pre-fixed threshold.

In a 215^{th} aspect according to any one of the previous two aspects, the other of the primary concentrate container (2) and the auxiliary concentrate container (3) houses a primary liquid concentrate (LC″), wherein the liquid concentrate mixing procedure includes a second filling step (FS″) of the primary liquid concentrate (LC″) to form a dialysis mixture (DiaM), wherein the control unit (24) is configured to execute the second filling step (FS″) of the primary liquid concentrate (LC″) after the first filling step (FS′) and the control unit (24) is configured to control the valves of the plurality of valves to set a primary concentrate filling path (F2) to fill the mixing container (11) with the primary liquid concentrate (LC″), particularly according to a pre-fixed nominal volume, during the filling step of the primary liquid concentrate the control unit (24) being configured to control the pump (8) to move the primary liquid concentrate (LC″) to the mixing container (11), in particular wherein during the second filling step (FS″) of the primary liquid concentrate (LC″), the control unit (24) is configured to operate a heater (23) on the main fluid line (4) to heat the primary liquid concentrate (LC″) flowing through the primary concentrate filling path up to a temperature suitable for conductivity sensing e.g., 37 °C.

In a 216^{th} aspect according to the previous aspect, the second filling step of the primary liquid concentrate (LC″) is followed by at least one second recirculation filling step (RS″) of the dialysis mixture (DiaM) formed by adding the primary liquid concentrate (LC″) to the first diluted liquid mixture (DM″) added with water, during the second the recirculation step (RS″) of the dialysis mixture (DiaM) the control unit (24) being configured to control the valves of the plurality of valves to set the mixing recirculation path (R1) and to mix the dialysis mixture (DiaM) into the mixing container (11) and form the dialysis fluid (FD), during the second recirculation step (RS″) of the dialysis mixture (DiaM) the control unit (24) being further configured to control the pump (8) to flow the dialysis mixture (DiaM) through the mixing recirculation path, in particular the control unit (24) is configured to perform an additional main fluid line concentration test step (MF-CTS″) to detect, by means of at least one conductivity meter (25, 26), conductivity variances of the dialysis mixture (DiaM) flowing through the mixing recirculation path (R1), the control unit (24) being configured to compare the conductivity variances detected in the dialysis mixture (DiaM) with a pre-selected threshold, and to interrupt the second recirculation step (RS″) of the dialysis mixture (DiaM) when the conductivity variance is below the pre-selected threshold.

In the summary section and in the description:
the mixing container outlet junction point (j1) is also referred to as first junction point;
the auxiliary concentrate container inlet junction point (j2) is also referred to as second junction point;
the auxiliary concentrate container outlet junction point (j3) is also referred to as third junction point;
the primary container inlet junction point (j4) is also referred to as fourth junction point;
the primary container outlet junction point (j5) is also referred to as fifth junction point;
the mixing container inlet junction point (j6) is also referred to as sixth junction point;
wherein the primary container junction point comprises both the primary container inlet junction point (j4) and the primary container outlet junction point (j5); the auxiliary concentrate container junction point comprises the auxiliary concentrate container inlet junction point (j2) and the auxiliary concentrate container outlet junction point (j3).

### Dry Concentrate Bag Aspects

The dry concentrate bag of any of the following aspects may be used in the medical fluid generation apparatus according to any of the previous aspects in any combination. The dry concentrate bag of any of the following aspects may be alternatively used in different medical fluid generation apparatus such as on-line/in-line medical fluid preparation apparatus or any other apparatus for preparing a dialysis fluid starting from at least one dry concentrate bag.

A 1st independent aspect is directed to a dry concentrate bag (100) for preparing a dialysis fluid comprising:
two plastic films (101, 102) welded together along welding lines (130) to define a housing space (103) for the dry concentrate;
a dry concentrate (104) housed within the housing space (103);
an inlet portion (110) comprising an inlet port (111) for receiving a fluid, the inlet port (111) being placed in correspondence of a bottom area in use conditions of the dry concentrate bag (100);
an outlet portion (120) comprising an outlet port (121) for letting the fluid mixed with the dry concentrate (104) to leave the housing space (103), the outlet port (121) being placed in correspondence of a top area in use conditions of the dry concentrate bag (100);
wherein the inlet portion (110) comprises a first inclined welding line (131) and a second inclined welding line (132) both emerging from the inlet port (111) and defining a lower cone portion (112) of the housing space (103), the inlet port (111) being placed at the lowest point of the lower cone portion (112),
wherein the outlet portion (120) comprises a third inclined welding line (133) and a fourth inclined welding line (134) both converging to the outlet port (121) and defining an upper cone portion (113) of the housing space (103), the outlet port (121) being placed at the highest point of the upper cone portion (113).

A 2^{nd} aspect is directed to a dry concentrate bag (100) for preparing a dialysis fluid comprising:
two plastic films (101, 102) welded together along welding lines (130) to define a housing space (103) for the dry concentrate;
a dry concentrate (104) housed within the housing space (103);
an inlet portion (110) comprising an inlet port (111) for receiving a fluid, the inlet port (111) being placed in correspondence of a bottom area in use conditions of the dry concentrate bag (100);
an outlet portion (120) comprising an outlet port (121) for letting the fluid mixed with the dry concentrate (104) to leave the housing space (103), the outlet port (121) being placed in correspondence of a top area in use conditions of the dry concentrate bag (100);
wherein the inlet portion (110) comprises a first inclined welding line (131) and a second inclined welding line (132) both emerging from the inlet port (111) and defining a lower cone portion (112) of the housing space (103), the inlet port (111) being placed at the lowest point of the lower cone portion (112).

In a 3^{rd} aspect according to the preceding aspect, the outlet portion (120) comprises a third inclined welding line (133) and a fourth inclined welding line (134) both converging to the outlet port (121) and defining an upper cone portion (113) of the housing space (103), the outlet port (121) being placed at the highest point of the upper cone portion (113).

A 4^{th} aspect is directed to a dry concentrate bag (100) for preparing a dialysis fluid comprising:
a body comprising a housing space (103) for the dry concentrate;
a dry concentrate (104) housed within the housing space (103);
an inlet portion (110) comprising an inlet port (111) for receiving a fluid, the inlet port (111) being placed in correspondence of a bottom area in use conditions of the dry concentrate bag (100);
an outlet portion (120) comprising an outlet port (121) for letting the fluid mixed with the dry concentrate (104) to leave the housing space (103), the outlet port (121) being placed in correspondence of a top area (122) in use conditions of the dry concentrate bag (100);
wherein the housing space (103) at the inlet portion (110) defines a lower cone portion (112), the inlet port (111) being placed at the lowest point of the lower cone portion (112).

In a 5^{th} aspect according to the preceding aspect, the housing space (103) at the outlet portion (120) defines an upper cone portion (113), the outlet port (121) being placed at the highest point of the upper cone portion (113).

A 6^{th} aspect is directed to a dry concentrate bag (100) for preparing a dialysis fluid comprising:
a plastic film defining two layers (101, 102) welded together along welding lines (130) to define a housing space (103) for the dry concentrate;
a dry concentrate (104) housed within the housing space (103);
an inlet portion (110) comprising an inlet port (111) for receiving a fluid, the inlet port (111) being placed in correspondence of a bottom area in use conditions of the dry concentrate bag (100);
an outlet portion (120) comprising an outlet port (121) for letting the fluid mixed with the dry concentrate (104) to leave the housing space (103), the outlet port (121) being placed in correspondence of a top area (122) in use conditions of the dry concentrate bag (100);
wherein the inlet portion (110) comprises a first inclined welding line (131) and a second inclined welding line (132) both emerging from the inlet port (111) and defining a lower cone portion (112) of the housing space (103), the inlet port (111) being placed at the lowest point of the lower cone portion (112).

In a 7^{th} aspect according to the preceding aspect, the outlet portion (120) comprises a third inclined welding line (133) and a fourth inclined welding line (134) both converging to the outlet port (121) and defining an upper cone portion (113) of the housing space (103), the outlet port (121) being placed at the highest point of the upper cone portion (113).

An 8^{th} aspect is directed to a manufacturing process for making a dry concentrate bag (100) comprising at least the following steps:
providing one or two plastic films defining plastic layers facing each other;
defining a first inclined welding line (131) and a second inclined welding line (132) both emerging from an inlet port (111) of the dry concentrate bag (100), said first inclined welding line (131) and said second inclined welding line (132) defining a lower cone portion (112) of a housing space (103) of the dry concentrate bag (100),
defining a third inclined welding line (133) and a fourth inclined welding line (134) both emerging from an outlet port (121) of the dry concentrate bag (100), said third inclined welding line (133) and said fourth inclined welding line (134) defining an upper cone portion (113) of said housing space (103) of the dry concentrate bag (100),
wherein said first, second, third and fourth inclined welding lines (131, 132, 133, 134) are made by a heat welding process wherein the two plastic layers are heated up to cause mutual welding,
in particular said dry concentrate bag being according any one of the preceding aspects.

In a 9^{th} aspect according to the preceding aspect, the steps of defining the first, second, third and fourth inclined welding lines (131, 132, 133, 134) comprise a step of pressing the two plastic films (101, 102) or layers one onto the other by a heat-sealing bar.

In a 10^{th} aspect according to any one of the preceding aspects, the two plastic film layers comprise:
a first film (101) and a second film (102) distinct and separated each other defining said two plastic film layers (101, 102); or
a single plastic film folded back on itself to define said two plastic film layers (101, 102).

In an 11^{th} aspect according to any one of the preceding aspects, the dry concentrate bag (100) further comprises a central portion (140) connecting the inlet portion (110) with the outlet portion (120).

In a 12^{th} aspect according to the preceding aspect, the central portion (140) comprises a fifth welding line (135) and a sixth welding line (136) opposed one to the other with respect to the housing space (103).

In a 13^{th} aspect according to the preceding aspect, the fifth welding line (135) is connected to the first inclined welding line (131) of the lower cone portion (112) and to the third inclined welding line (133) of the upper cone portion (113).

In a 14^{th} aspect according to any one of the preceding two aspects, the sixth welding line (136) is connected to the second inclined welding line (132) of the lower cone portion (112) and to the fourth inclined welding line (134) of the upper cone portion (113).

In a 15^{th} aspect according to any one of the preceding aspects, the first, second, third, fourth, fifth and sixth welding lines delimit and define the housing space (103).

In a 16^{th} aspect according to any one of the preceding aspects from 12, the fifth welding line (135) and the sixth welding line (136) are substantially parallel one another to define a rectangular portion of the housing space (103).

In a 17^{th} aspect according to any one of the preceding aspects from 11, the central portion is substantially rectangular or square shaped.

In an 18^{th} aspect according to any one of the preceding aspects, the dry concentrate bag (100) is substantially symmetric with respect to a longitudinal axis (A) passing through the inlet port (111) and the outlet port (121).

In a 19^{th} aspect according to any one of the preceding aspects, the dry concentrate bag extends in length along a longitudinal axis (A), and in width along a transversal axis (B) orthogonal to the longitudinal axis (A).

In a 20^{th} aspect according to any one of the preceding aspects, the first inclined welding line (131) defines an angle (α) with respect to a longitudinal axis (A) passing through the inlet port (111) and the outlet port (121), said angle (α) being smaller than a corresponding angle (B) defined by the third inclined welding line (133) of the upper cone portion (113) with respect to the longitudinal axis (A).

In a 21^{st} aspect according to any one of the preceding aspects, the second inclined welding line (132) defines an angle (α′) with respect to a longitudinal axis (A) passing through the inlet port (111) and the outlet port (121), said angle (α′) being smaller than a corresponding angle (β′) defined by the fourth inclined welding line (134) of the upper cone portion (113) with respect to the longitudinal axis (A).

In a 22^{nd} aspect according to any one of the preceding aspects, the first inclined welding line (131) defines an angle (α) with respect to a longitudinal axis (A) passing through the inlet port (111) and the outlet port (121), said angle (α) being smaller than a corresponding angle (β′) defined by the fourth inclined welding line of the upper cone portion (113) with respect to the longitudinal axis (A).

In a 23^{rd} aspect according to any one of the preceding aspects, the second inclined welding line (132) defines an angle (α′) with respect to a longitudinal axis (A) passing through the inlet port (111) and the outlet port (121), said angle (α′) being smaller than a corresponding angle (β) defined by the third inclined welding line (133) of the upper cone portion (113).

In a 24^{th} aspect according to any one of the preceding aspects, the first inclined welding line (131) and/or the second inclined welding line (132) define an angle (α, a′) with respect to a longitudinal axis (A) passing through the inlet port (111) and the outlet port (121), said angle being smaller than 60°, in particular smaller than 45° and even in more detail smaller than 30°.

In a 25^{th} aspect according to any one of the preceding aspects, the third inclined welding line (133) and/or the fourth inclined welding line (134) define an angle with respect to a longitudinal axis (A) passing through the inlet port (111) and the outlet port (121), said angle being larger than 30°, in particular larger than 45° and even in more detail larger than 60°.

In a 26^{th} aspect according to any one of the preceding aspects, the lower cone portion (112) does not include dead volumes near the inlet port (111).

In a 27^{th} aspect according to any one of the preceding aspects, the upper cone portion (113) does not include dead volumes near the outlet port (121).

In a 28^{th} aspect according to any one of the preceding aspects, the housing space (103) does not include dead volumes.

In a 28^{th} bis aspect according to any one of the preceding aspects, the dry concentrate bag (100) is configured to be hanged, for example to a hook.

In a 29^{th} aspect according to any one of the preceding aspects, the dry concentrate bag (100) comprises one or more hanging elements (125) placed on the upper cone portion (113), such as holes for hooks or hangers, in particular wherein said one or more hanging elements (125) are configured to hang the dry concentrate bag (100).

In a 30^{th} aspect according to any one of the preceding aspects, the plastic films are configured to withstand temperatures of at least 70 °C, in particular to withstand temperatures of 80-90 °C.

In a 31^{st} aspect according to any one of the preceding aspects, the housing space (103) is defined exclusively by the two plastic films that are flat films.

In a 32^{nd} aspect according to any one of the preceding aspects, the housing space (103) is configured to house at least 1000 ml of fluid and optionally at least 1250 ml received from the inlet port.

In a 33^{rd} aspect according to any one of the preceding aspects, the dry concentrate bag (100) further comprises a first filter (150) placed to filter (all) fluid exiting from the outlet port (121), said first filter (150) being in particular configured to withhold the dry concentrate (104) in solid form, optionally said filter being placed on an outlet tube connected to the outlet port.

In a 34^{th} aspect according to any one of the preceding aspects, the dry concentrate bag (100) further comprises a second filter (alternatively a bottom powder or granulate stop) (151) placed in correspondence of the inlet port (111) to prevent powder to pass through the inlet port (111).

In a 35^{th} aspect according to any one of the preceding aspects, the dry concentrate (104) housed in the housing space (103) is in the form of a powder or a granulate.

In a 36^{th} aspect according to any one of the preceding aspects, the dry concentrate (104) includes an osmotic agent, particularly glucose monohydrate.

In a 37^{th} aspect according to any one of the preceding aspects, the dry concentrate comprises at least 80% of glucose monohydrate and particularly 100% of glucose monohydrate.

In a 38^{th} aspect according to any one of the preceding aspects, the housing space (103) and a dry concentrate (104) quantity housed in the housing space in dry form are such to allow, when in use, a dilution with water to achieve a concentration in the liquid concentrate between 15% and 70% w/v.

In a 39^{th} aspect according to any one of the preceding aspects, the first inclined welding line (131) and the second inclined welding line (132) are fluid tight.

In a 40^{th} aspect according to any one of the preceding aspects, the second inclined welding line (132) and the third inclined welding line (133) are fluid tight.

In a 41^{st} aspect according to any one of the preceding aspects, the fifth welding line (135) and the sixth welding line (136) are fluid tight.

In a 42^{nd} aspect according to any one of the preceding aspects, the welding lines (130) are fluid tight.

In a 43^{rd} aspect according to any one of the preceding aspects, the housing space (103) communicates with external environment only through the inlet port (111) and through the outlet port (121).

In a 44^{th} aspect according to any one of the preceding aspects, the housing space (103) at the lower cone portion (112) is “V” shaped, in particular in a condition wherein the housing space (103) is substantially empty.

In a 45^{th} aspect according to any one of the preceding aspects, the housing space (103) at the upper cone portion (113) is “V” shaped, in particular in a condition wherein the housing space (103) is substantially empty.

In a 46^{th} aspect according to any one of the preceding aspects, the housing space (103) at the lower cone portion (112) is “cone” shaped, in particular in a condition wherein the housing space (103) is filled with the dry concentrate.

In a 47^{th} aspect according to any one of the preceding aspects, the housing space (103) at the upper cone portion (113) is “cone” shaped, in particular in a condition wherein the housing space (103) is filled with the dry concentrate (104) and optionally with medical fluid.

In a 48^{th} aspect according to any one of the preceding aspects, the upper cone portion (113) is three dimensional.

In a 49^{th} aspect according to any one of the preceding aspects, the lower cone portion (112) is tridimensional.

In a 50^{th} aspect according to any one of the preceding aspects, the dry concentrate bag is made of flexible plastic film.

In a 51^{st} aspect according to any one of the preceding aspects, the housing space (103) is configured to increase a volume thereof between a condition wherein the housing space (103) is empty, and a condition wherein the housing space (103) houses the dry concentrate (104) and/or a fluid.

In a 52^{nd} aspect according to any one of the preceding aspects, the first inclined welding line (131) is straight.

In a 53^{rd} aspect according to any one of the preceding aspects, the second inclined welding line (132) is straight.

In a 54^{th} aspect according to any one of the preceding aspects, the third inclined welding line (133) is straight.

In a 55^{th} aspect according to any one of the preceding aspects, the fourth inclined welding line (134) is straight.

In a 56^{th} aspect according to any one of the preceding aspects, the fifth welding line

(135) is straight.

In a 57^{th} aspect according to any one of the preceding aspects, the sixth welding line (136) is straight.

In a 58^{th} aspect according to any one of the preceding aspects, the smallest sizes of the housing space (103) are located at the lower cone portion (112) and at the upper cone portion (113).

In a 59^{th} aspect according to any one of the preceding aspects, the dry concentrate bag (100) further comprises a central portion (140) connecting the inlet portion (110) with the outlet portion (120), the central portion (140) comprising a fifth welding line (135) and a folding line (137) opposed one to the other with respect to the housing space (103), the fifth welding line (135) being connected to the first inclined welding line (131) of the lower cone portion (112) and to the third inclined welding line (133) of the upper cone portion (113), the folding line (137) being connected to the second inclined welding line (132) of the lower cone portion (112) and to the fourth inclined welding line (134) of the upper cone portion (113), in particular the first, second, third, fourth, fifth welding lines and the folding line (137) delimiting and defining the housing space (103).

In a 60^{th} aspect according to any one of the preceding aspects, the fifth welding line (135), and the folding line (137) are substantially parallel to one another to define a rectangular portion of the housing space (103).

In a 61^{st} aspect according to any one of the preceding aspects, the dry concentrate (104) comprises buffer and electrolyte, in particular said dry concentrate being directed to peritoneal dialysis or to hemo-dialysis.

In a 62^{nd} aspect according to any one of the preceding aspects, the dry concentrate (104) comprises at least one substance in the group between NaCl, NaLa, CaCl₂), MgCl₂, in particular wherein at least the 90%, in particular at least the 95%, of the dry concentrate consists of NaCl and a buffer, such as sodium lactate (NaLa).

In a 62^{nd} bis aspect according to any one of the preceding two aspects, the housing space (103) and a dry concentrate (104) quantity housed in the housing space in dry form are such to allow, when in use, a dilution with water to achieve a concentration in the liquid concentrate between 10% to 30% w/v.

In a 63^{rd} aspect according to any one of the preceding aspects, the dry concentrate (104) comprises NaCl and NaLa.

In a 64^{th} aspect according to any one of the preceding aspects, the dry concentrate (104) comprises NaCl in a concentration higher than 45%, in particular higher than 50%, in particular comprised between 50% and 55%.

In a 65^{th} aspect according to any one of the preceding aspects, the dry concentrate (104) comprises NaLa in a concentration higher than 35%, in particular higher than 40%, in particular comprised between 40% and 50%.

In a 66^{th} aspect according to any one of the preceding aspects, in case all powders are anhydrous, the dry concentrate (104) comprises the following substance concentrations:

| Component | Weight percentage |
|---|---|
| NaCl | 53.65% |
| CaCl₂ | 1.38% |
| MgCl₂ | 0.24% |
| NaLa | 44.73% |

Additionally, any of the above aspects, or portions thereof, and/or any of the features, functionality, and alternatives described in connection with any one or more of FIGS. 1A to 1Q may be combined with any of the features, functionality and alternatives described in connection with any other of FIGS. 2 to 4.

It is accordingly an advantage of the present disclosure to provide a system that makes fresh dialysis fluid that may be housed or stored in the mixing container and used later on.

It is another advantage of the present disclosure to provide a system that makes fresh dialysis fluid for a patient, for example for home dialysis, only requiring the use of liquid and/or dry concentrates thereby reducing the medical fluid bag handling.

It is a further advantage of the present disclosure to provide a system that makes fresh dialysis fluid with a simple hydraulic circuit requiring only a single pump working on a main fluid line and capable of managing the various medical fluid production steps.

It is yet a further advantage of the present disclosure to provide a system that makes fresh dialysis fluid which may be placed in containers for later use.

Moreover, it is an advantage to make fluids, such as PD fluids, CRRT fluids, lactated ringers and the like closer in time and distance to a point of use for the patient, which lessens the amount of supplies that have to be stored, e.g., at a patient's home.

It is still a further advantage to prepare PD fluid using one or more concentrates.

Another advantage of the present disclosure is to provide a durable fluid circuit that is therefore reusable and reduces the efforts on the patient when preparing the medical fluid as e.g., there is no need to set up disposable lines and/or dress the medical apparatus with fluid flow paths. Further, less material waste is generated.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1 is a schematic view of a fluid circuit of a medical fluid generation apparatus according to a first embodiment of the present invention;

FIG. 1A is a schematic view of the fluid circuit shown in FIG. 1, with an air evacuation flow path properly highlighted;

FIG. 1B is a schematic view of the fluid circuit shown in FIG. 1, with a main fluid line prime path properly highlighted;

FIG. 1C is a schematic view of the fluid circuit shown in FIG. 1, with a recirculation priming path properly highlighted;

FIG. 1D is a schematic view of the fluid circuit shown in FIG. 1, with a first dissolution liquid fill path properly highlighted;

FIG. 1E is a schematic view of the fluid circuit shown in FIG. 1, with a second dissolution liquid fill path properly highlighted;

FIG. 1F is a schematic view of the fluid circuit shown in FIG. 1, with a first concentrate recirculation path properly highlighted;

FIG. 1G is a schematic view of the fluid circuit shown in FIG. 1, with an auxiliary air evacuation properly highlighted;

FIG. 1H is a schematic view of the fluid circuit shown in FIG. 1, with a first auxiliary recirculation priming path properly highlighted;

FIG. 1I is a schematic view of the fluid circuit shown in FIG. 1, with a first auxiliary dissolution liquid fill path properly highlighted;

FIG. 1J is a schematic view of the fluid circuit shown in FIG. 1, with a second auxiliary dissolution liquid fill path properly highlighted;

FIG. 1K is a schematic view of the liquid circuit shown in FIG. 1, with a second concentrate recirculation path properly highlighted;

FIG. 1L is a schematic view of the liquid circuit shown in FIG. 1, with a mixing container drain path properly highlighted;

FIG. 1M is a schematic view of the liquid circuit shown in FIG. 1, with a mixing container filling path properly highlighted;

FIG. 1N is a schematic view of the liquid circuit shown in FIG. 1, with an auxiliary concentrate filling path properly highlighted;

FIG. 1O is a schematic view of the liquid circuit shown in FIG. 1, with a water filling path properly highlighted;

FIG. 1P is a schematic view of the liquid circuit shown in FIG. 1, with a mixing recirculation path properly highlighted;

FIG. 1Q is a schematic view of the liquid circuit shown in FIG. 1, with a primary concentrate filling path properly highlighted.

FIG. 2 is a schematic view of a fluid circuit of a medical fluid generation apparatus according to a second embodiment of the present invention;

FIG. 3 is a schematic view of a fluid circuit of a medical fluid generation apparatus according to a third embodiment of the present invention;

FIG. 4 is a schematic view of a fluid circuit of a medical fluid generation apparatus according to a fourth embodiment of the present invention;

FIG. 4A is a schematic view of a fluid circuit of a medical fluid generation apparatus according to a fifth embodiment of the present invention;

FIG. 4B is a schematic view of a fluid circuit of a medical fluid generation apparatus according to a sixth embodiment of the present invention;

4C is a schematic view of a fluid circuit of a medical fluid generation apparatus according to a seventh embodiment of the present invention

FIG. 5 is a generic block diagram concerning a dry concentrate dissolution procedure which may be performed with the medical fluid generation apparatus shown in FIGS. 1, 2, 3 and 4;

FIG. 6 is a detailed diagram block concerning the dry concentrate dissolution procedure of FIG. 5 which may be performed with the medical fluid generation apparatus shown in FIGS. 1, 2, 3 and 4;

FIG. 7 is a generic block diagram concerning a liquid concentrate mixing procedure which may be performed with the medical fluid generation apparatus shown in FIGS. 1, 2, 3 and 4;

FIG. 8 is a second generic diagram block concerning the liquid concentrate mixing procedure of FIG. 7 which may be performed with the medical fluid generation apparatus shown in FIGS. 1, 2, 3 and 4;

FIG. 9 is a detailed diagram block concerning the liquid concentrate mixing procedure of FIGS. 6 and 7 which may be performed with the medical fluid generation apparatus shown in FIGS. 1, 2, 3 and 4;

FIGS. 10, 11 and 14 show a dry bag concentrate according to an embodiment of the present description;

FIG. 10A shows an alternative embodiment of a dry bag concentrate bag;

FIGS. 12 and 13 show respectively a laterally and a longitudinally sectioned dry bag of FIGS. 10 and 11;

FIG. 15 shows a dry bag concentrate according to a further embodiment of the present description;

FIGS. 16 and 17 show respectively a laterally and a longitudinally sectioned dry bag of FIG. 15;

FIGS. 18A-18C illustrate a dissolving sequence of the dry concentrate;

FIG. 19 shows a dry bag concentrate according to a further embodiment of the present description;

FIGS. 20 and 21 show respectively a laterally and a longitudinally sectioned dry bag of FIG. 19.

### DETAILED DESCRIPTION

### Medical Fluid Generation Apparatus

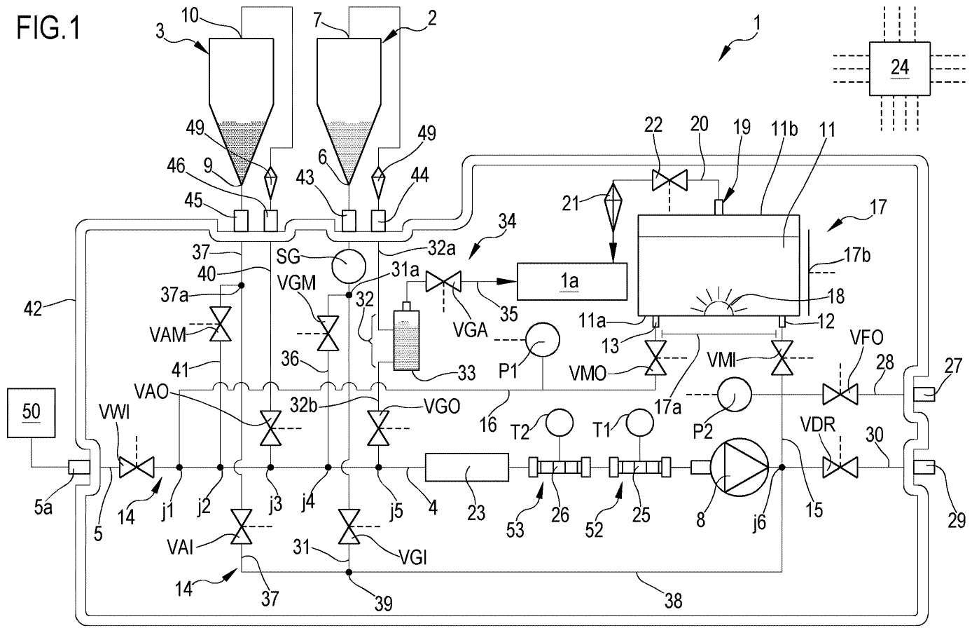

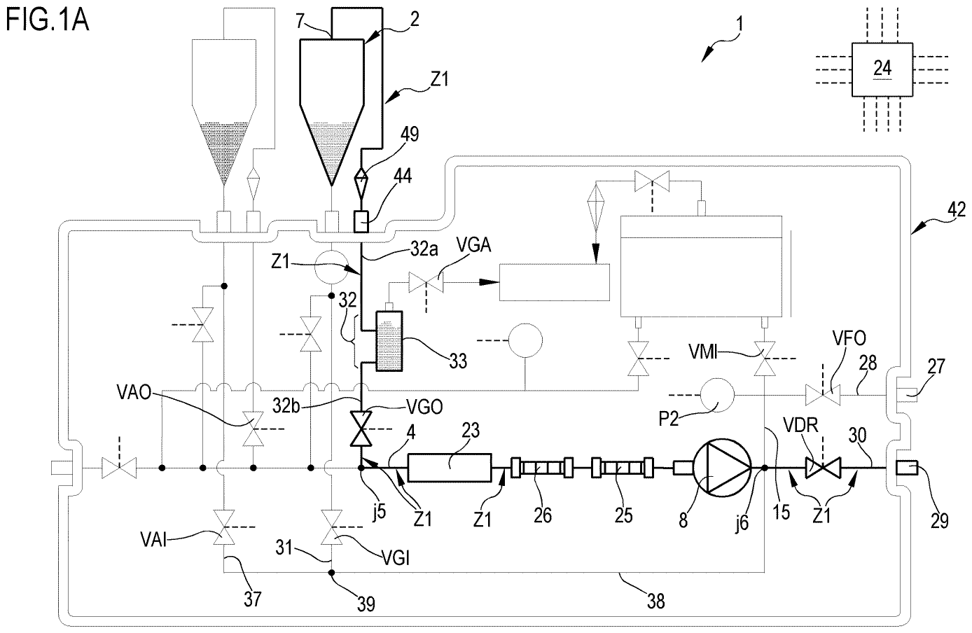

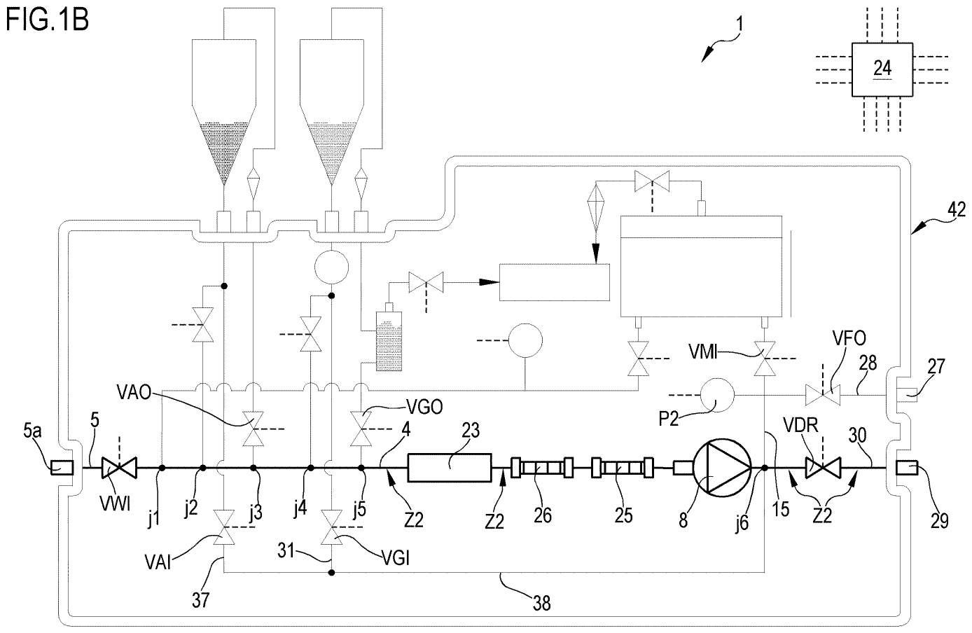

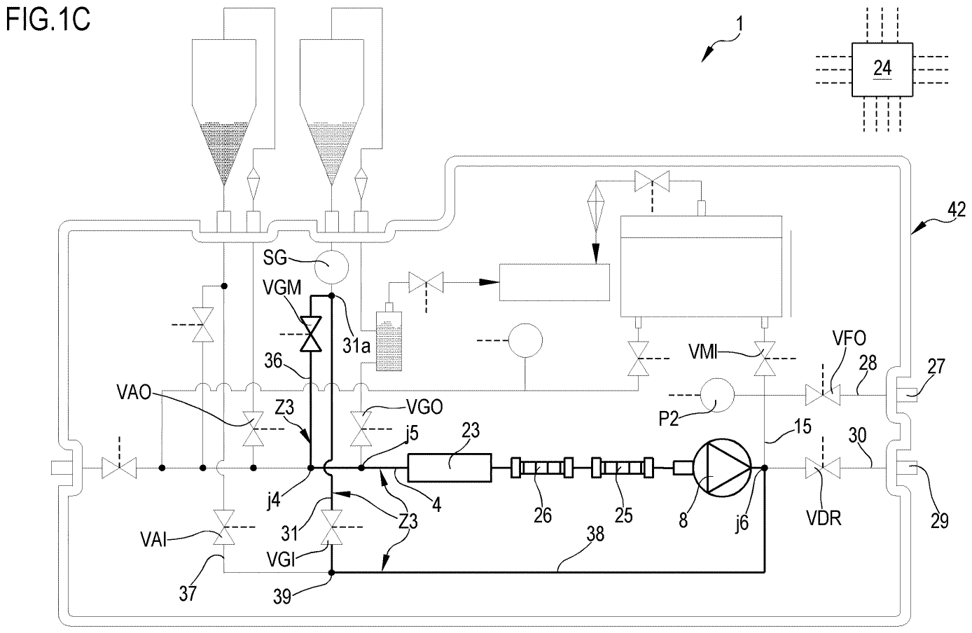

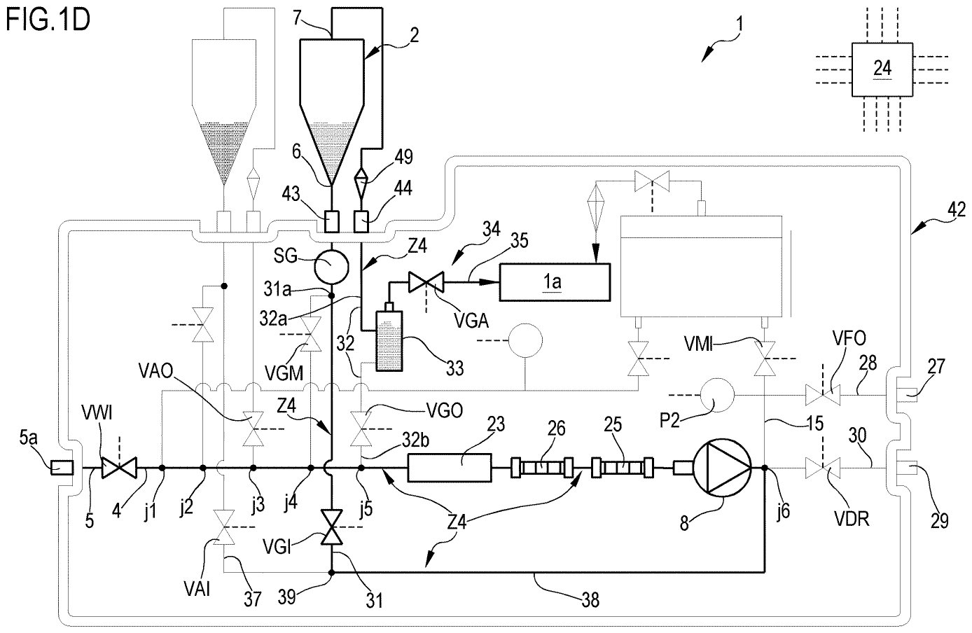

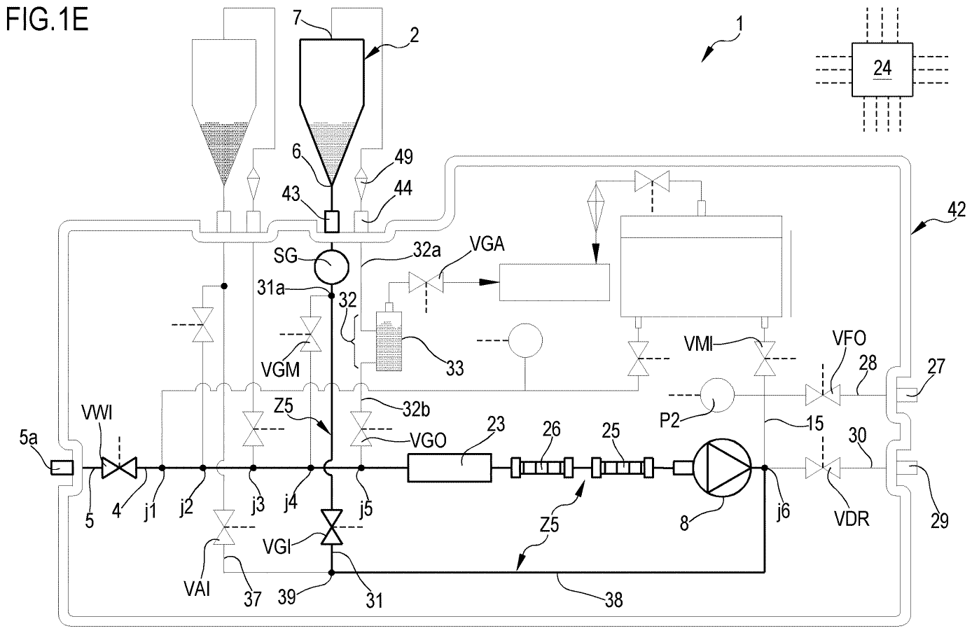

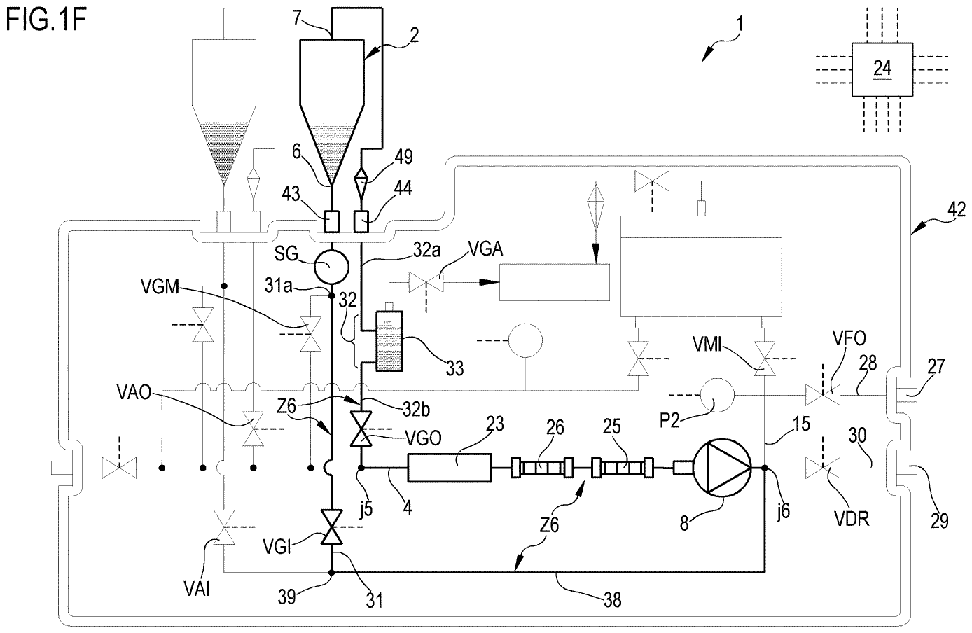

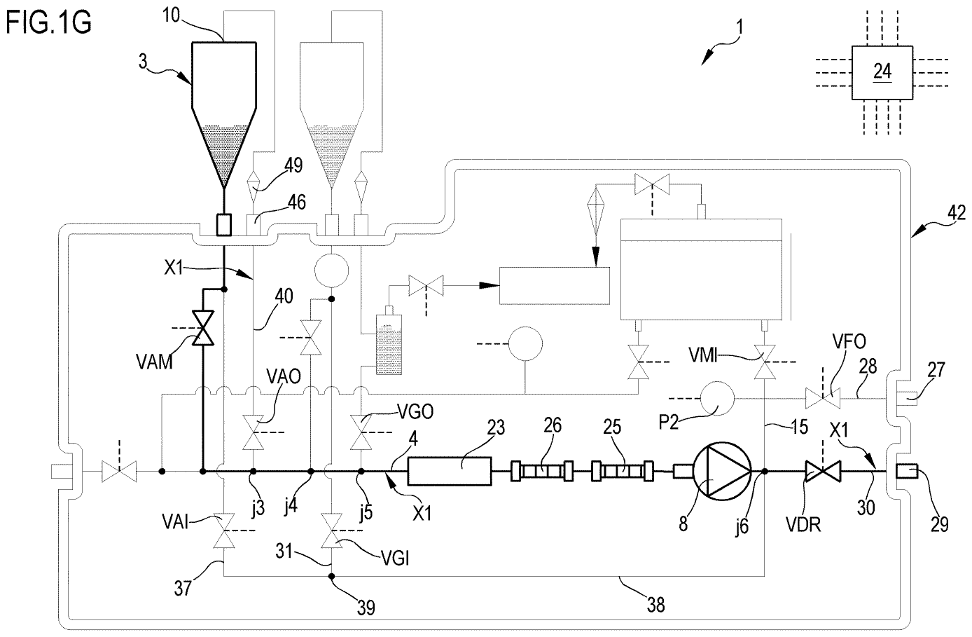

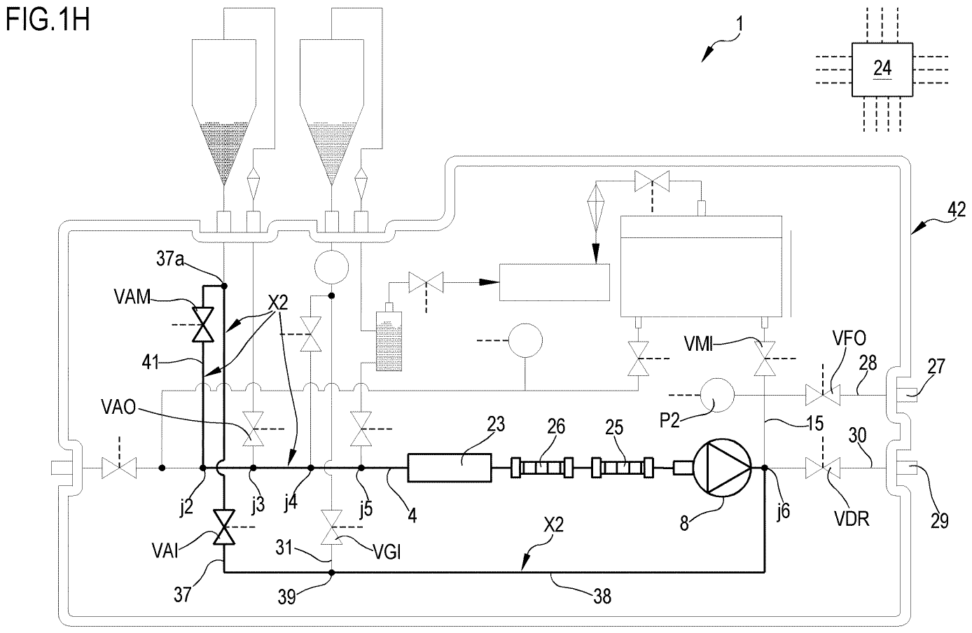

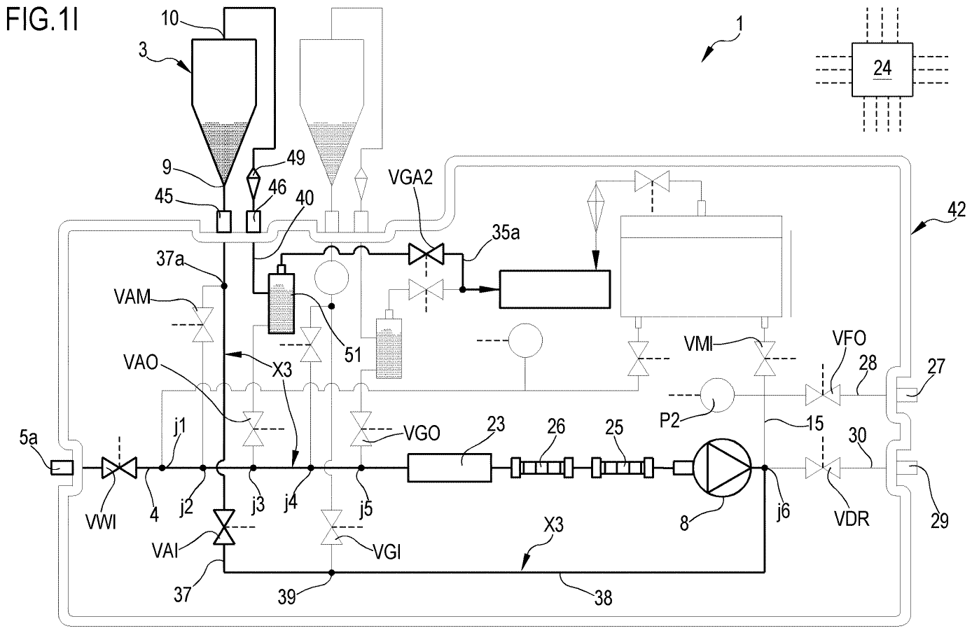

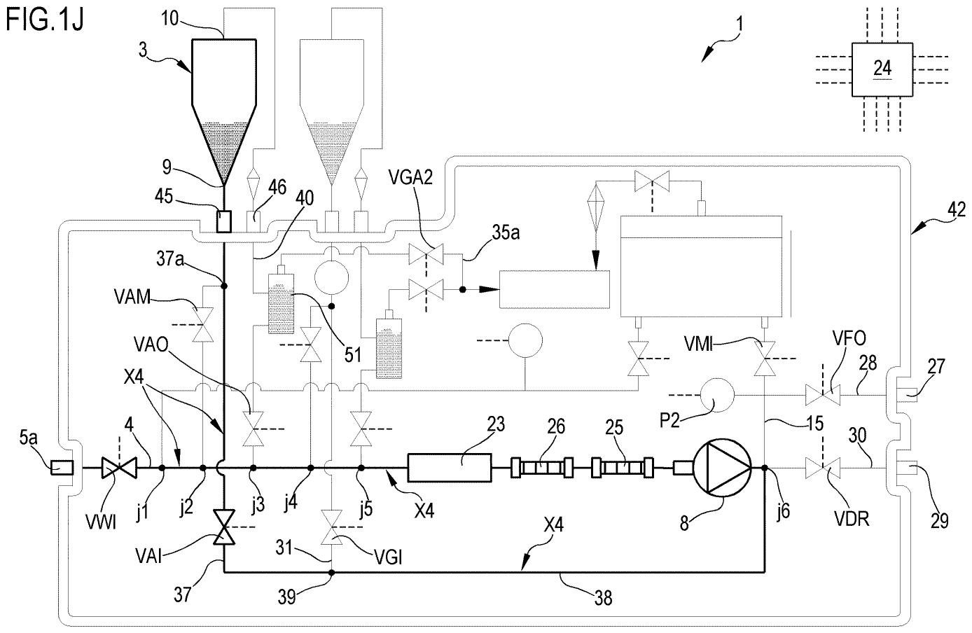

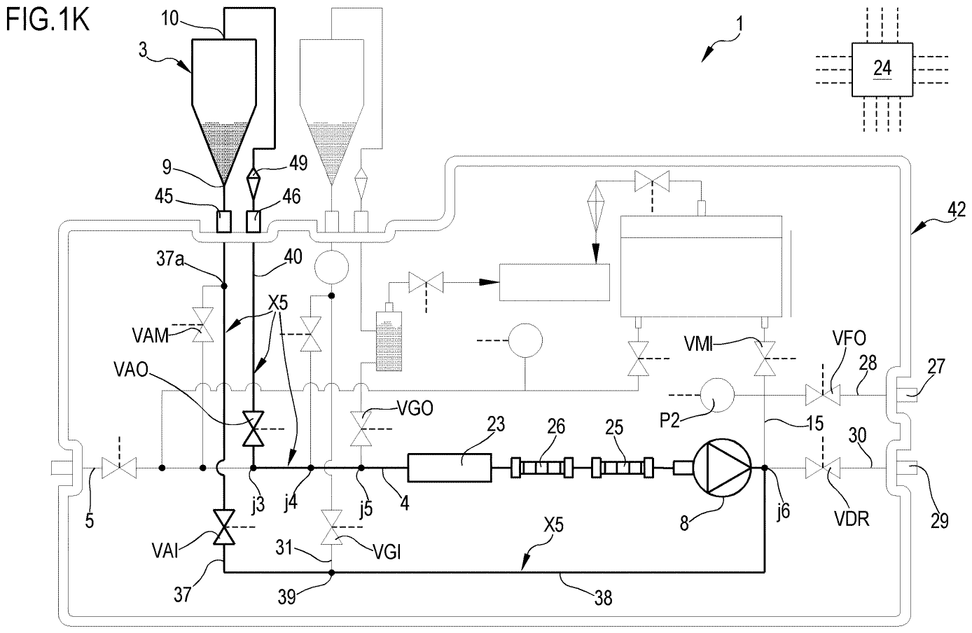

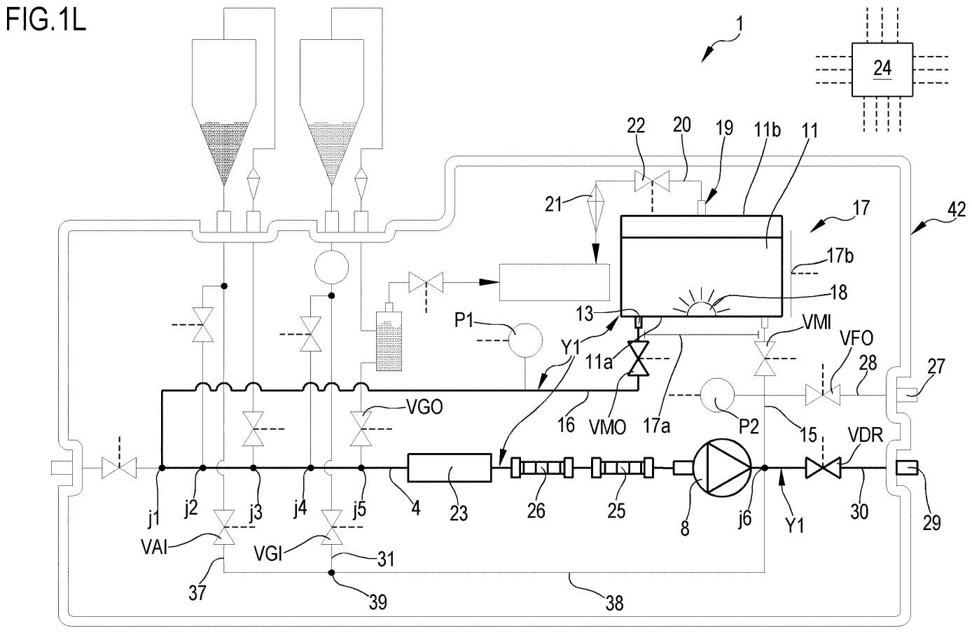

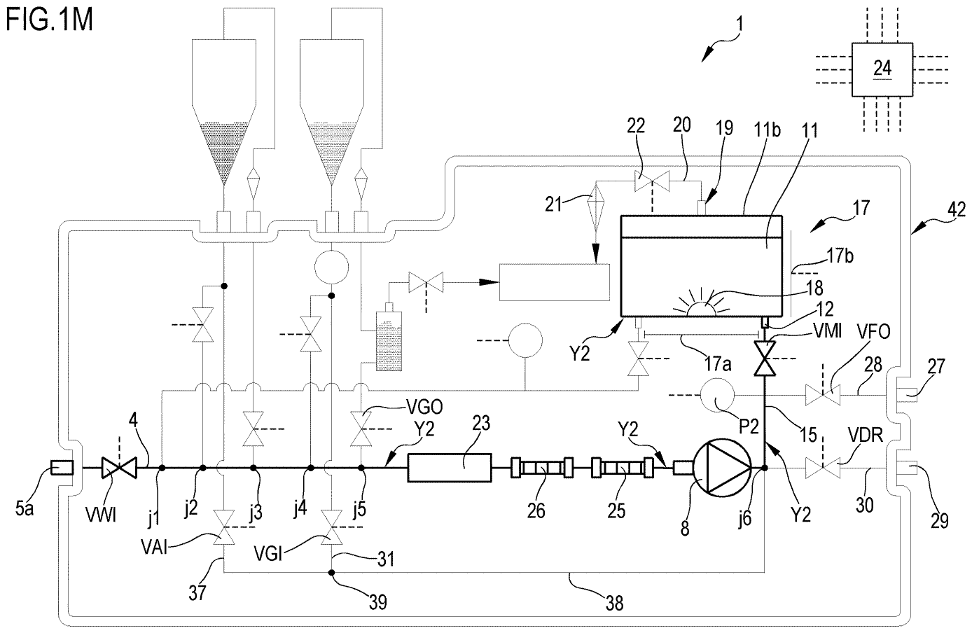

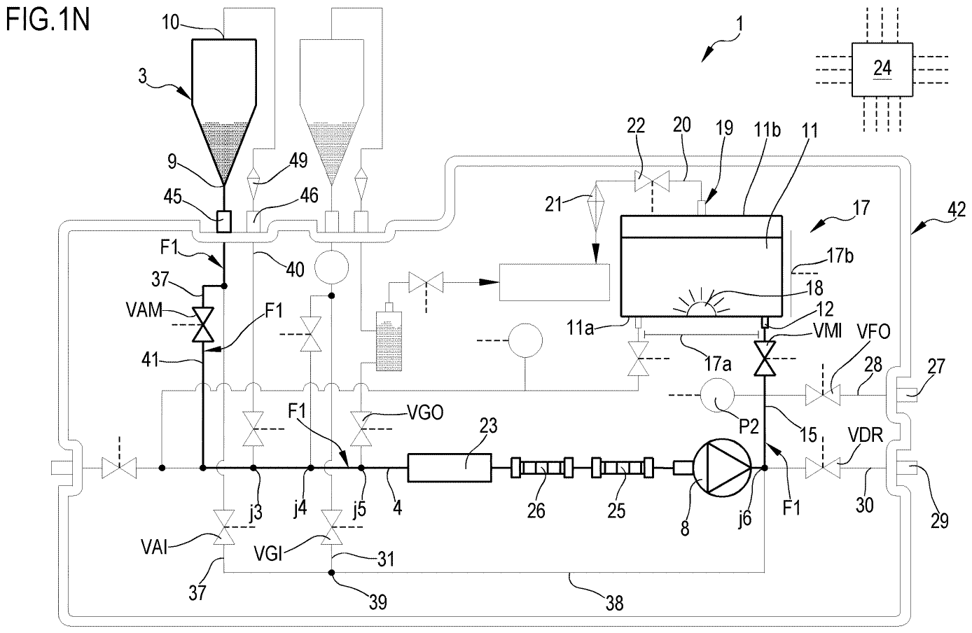

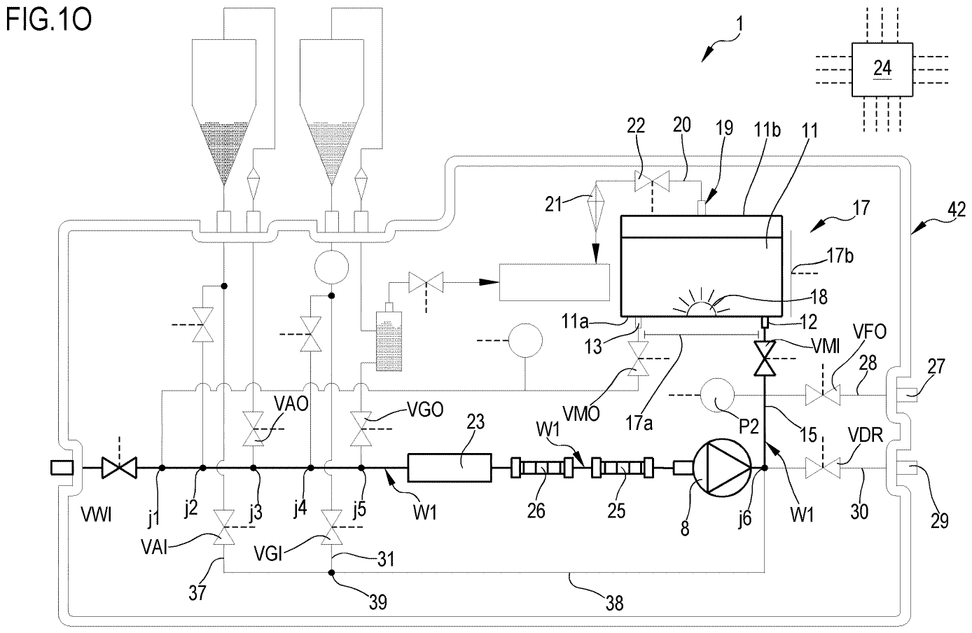

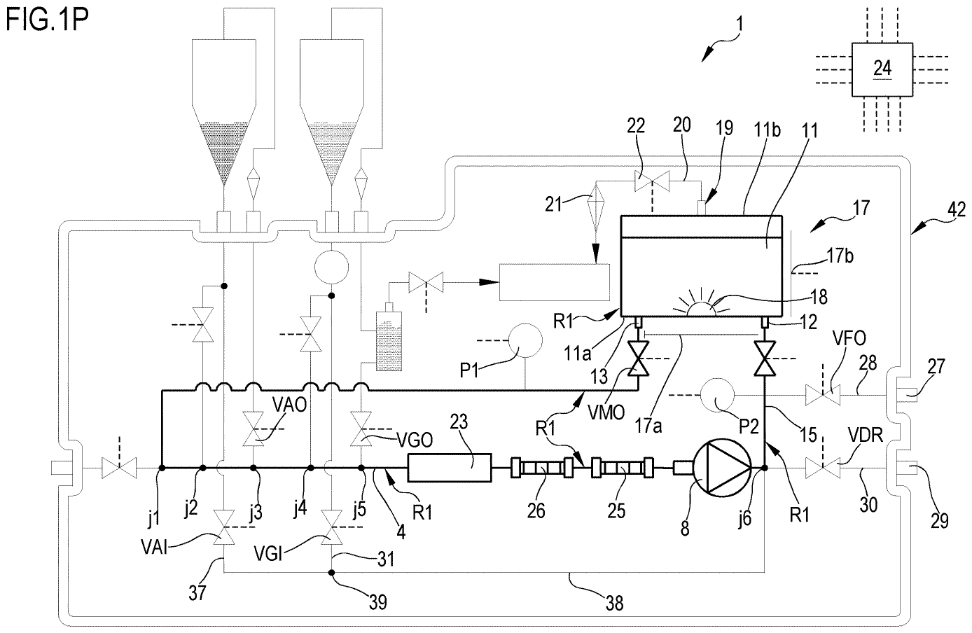

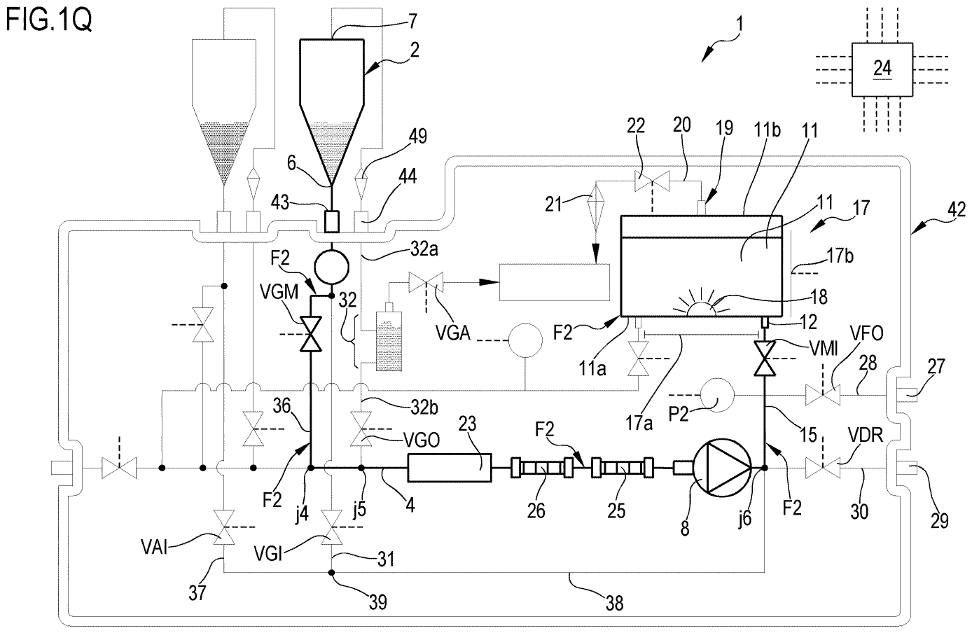

Considering the embodiments shown in FIGS. 1 to 4C, a medical fluid generation apparatus 1 for preparing a medical fluid such as dialysis fluid, and in particular PD fluid or HD fluid, is schematically outlined.

As it is possible to see from FIGS. 1 to 4, the medical fluid apparatus 1 comprises a supporting structure 42 defining a housing for containing different inner parts and/or inner elements and/or inner components of the medical fluid apparatus 1. Moreover, the supporting structure 42 provides different external connecting areas which allow one or more external elements and/or external components of the medical fluid apparatus 1 to operatively connect to the aforementioned inner parts and/or inner elements and/or inner components of medical fluid apparatus 1 and interact with them in order to perform different processes and functions which will be described throughout of this description.

Always referring to the embodiments shown in FIGS. 1 to 4, the supporting structure 42 houses a fluid circuit 14 which includes a main fluid line 4 provided with an inlet point 5 for receiving water. The supporting structure encloses the fluid circuit 14, valves connected to the fluid circuit, pump and other internal components (as further detailed below) thereby protecting them from direct access by the user. Further, the fluid circuit 14 is durable (i.e., non-disposable) and is used each time it is necessary to produce medical fluid, in case after proper disinfection. Of course, in a different and alternative embodiment, the fluid circuit may be a disposable circuit.

The supporting structure 42 has an entrance 5a for connection to a water source. Depending on the specific embodiment of the medical fluid generation apparatus 1, either already pure water is fed to the apparatus through the entrance 5a or water that needs to be purified is provided. Indeed, the medical fluid generation apparatus 1 may or may not be provided with a purification device 50.

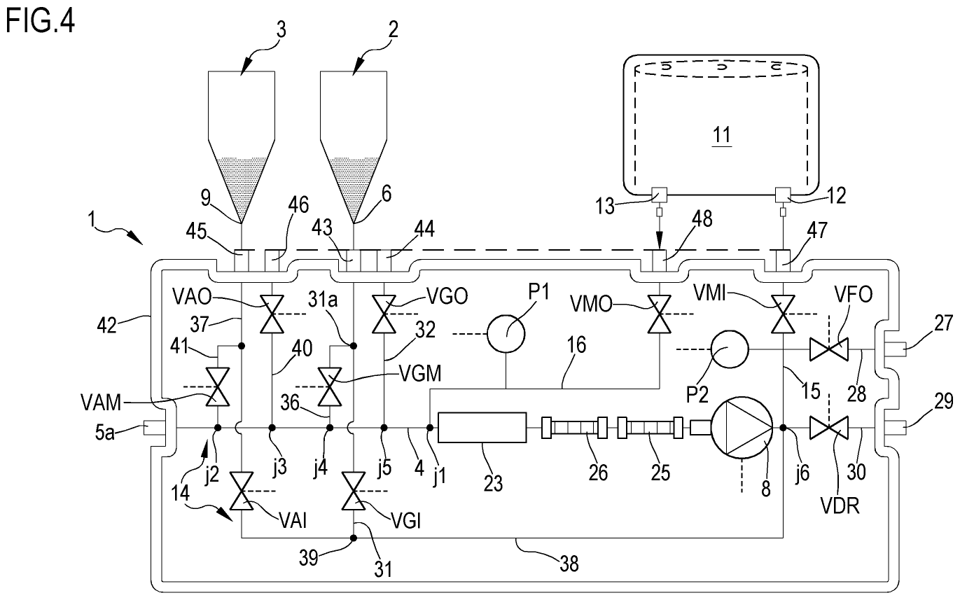

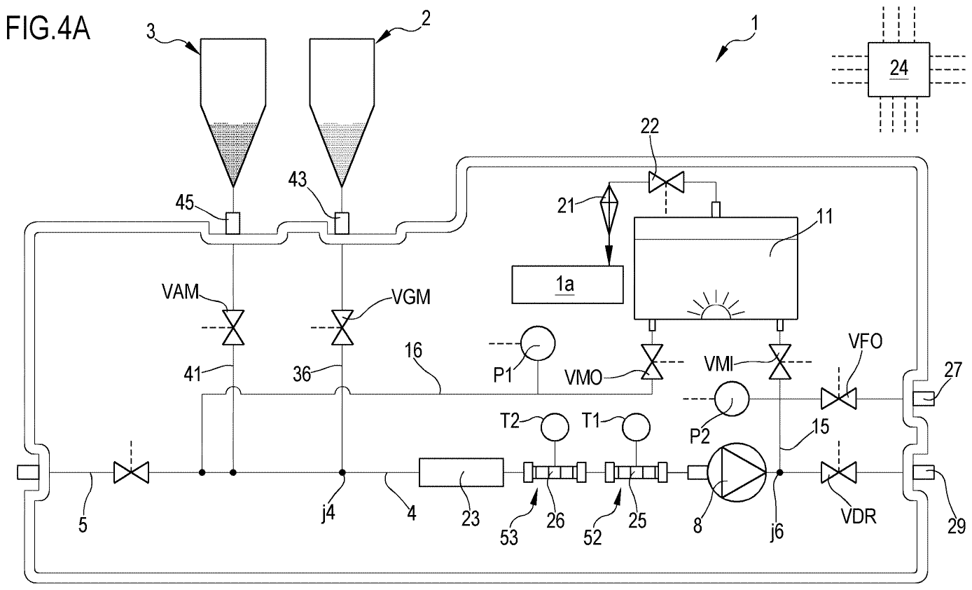

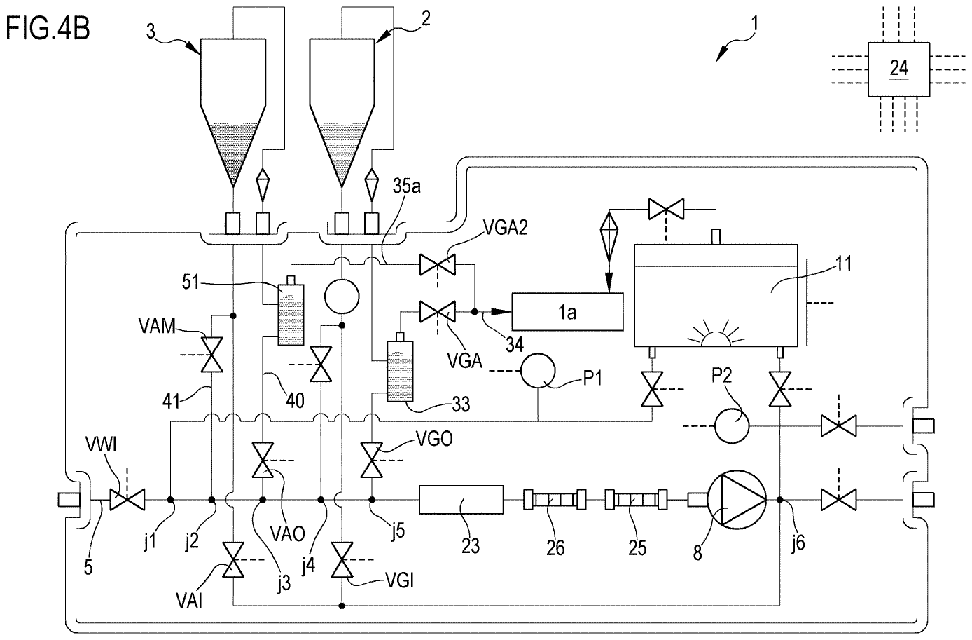

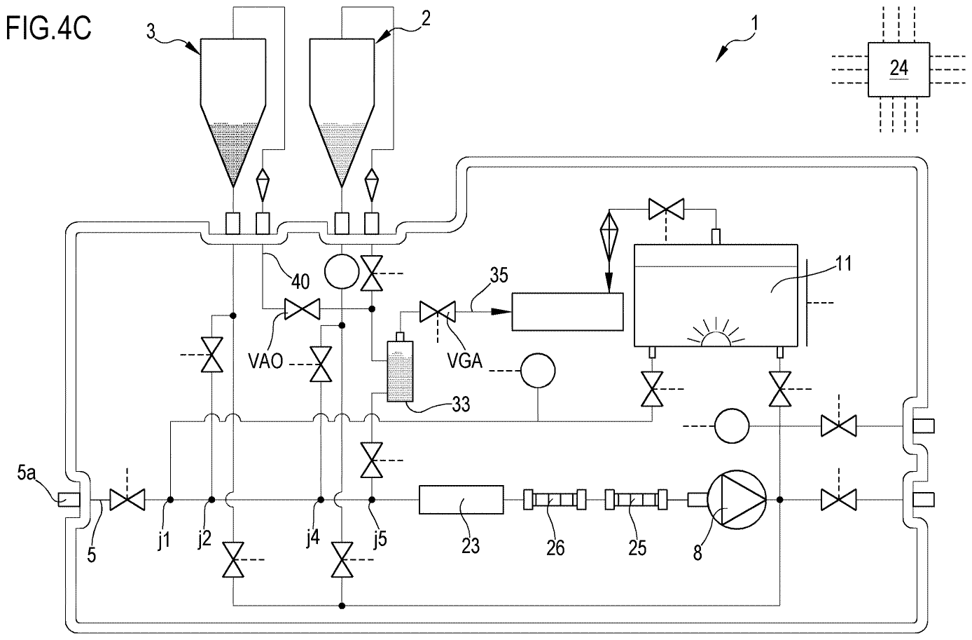

Independent of the configuration, the inlet point 5 of the main fluid line 4 receives pure water, e.g., water that has been treated to remove impurities, contaminants, chemicals, and dangerous microorganisms, as well as solutes. Pure water may be obtained by distillation, deionization, reverse osmosis, or other suitable process and that meets the definition of pure water in the relevant Pharmacopeia. For example, pure water may be produced with a known reverse osmosis device. In one example (see FIG. 4A), a purification device 50 (e.g., the mentioned reverse osmosis device) may be included inside the supporting structure 42, upstream the inlet point 5. Water, such as tap water, enters the supporting structure through an entrance 5a for water and reaches the purification device 50. Water is pure and sent towards the inlet point 5.

In an alternative embodiment (see for example FIG. 1), the pure water arrives directly at entrance 5a, in particular from an external supply source (not shown in the attached figures) that may be chosen among different known supply sources. Preferably, the external supply source may comprise a water purification equipment 50 configured to provide pure water to the entrance 5a and consequently to the inlet point 5 for receiving pure water. The water purification equipment includes a central water purification station configured to feed the entrance 5a and the inlet point 5 or a standalone water purifier configured to feed the entrance 5a and the inlet point 5. In order to allow the external supply source to feed the inlet point 5 with the pure water, the supporting structure 42 is externally provided with the proper entrance connection. In the embodiment of FIG. 1, entrance 5a and inlet point 5 may be coincident. Notably, also embodiments of FIGS. 1, 2, 3, 4, 4B, and 4C may include a purification device 50 downstream the entrance 5a as an alternative design.

In all the embodiments, the supporting structure 42 also houses a pump 8, preferably a volumetric pump 8, configured to operate on the main fluid line 4 to circulate at least one fluid, at least in the main fluid line 4 of the fluid circuit 14. As apparent in the following description, one single pump 8 may be sufficient for the working of the apparatus. More in detail, such pump 8 always moves the fluid in the same direction (i.e., is never operated in reverse pumping).

The supporting structure 42 houses a plurality of valves configured to operate on the fluid circuit 14 to set different fluid paths for the fluid inside the fluid circuit 14, in particular according to different processes and operations to be executed by the medical fluid generation apparatus 1. In general a valve has an open condition allowing fluid to pass through and a closed condition preventing any fluid passage. Any kind of valve suitable for the task may be used, such as an on/off valve, a pinch valve, a cassette valve, a proportional valve, etc.,

The medical fluid generation apparatus 1 also comprises a control unit 24 configured to operate at least the pump 8 and/or the plurality of valves in order to command and manage the configuration of the plurality of valves and establish the fluid paths to be followed by the fluid or the fluids inside the fluid circuit 14. Advantageously, the control unit 24 is electrically and/or electronically connected to the plurality of valves and the other parts and/or elements and/or components of the medical generation apparatus 1, through a plurality of known electrically and/or electronically connections not shown in the attached figures. Alternatively, the control unit 24 may be connected to the plurality of valves and the other parts and/or elements and/or components of the medical generation apparatus 1, through a wireless connection system that needs at least one operatively interposed unit able to emit and receives one or more signals by using different communication protocols that are known in the art.

As shown in FIGS. 1 to 4 and 4B/4C, the supporting structure 42 is provided with four container connectors/accesses 43, 44, 45, 46 in fluid communication with the inner fluid circuit 14 in order to allow one or more fluids running along the fluid circuit 14 to be delivered to at least one external portion of the fluid circuit 14; the container connectors/accesses 43, 44, 45, 46 may also serve to deliver fluid from a connected component to the inside fluid circuit.

In particular, the supporting structure 42 has a first primary container access 43 and a second primary container access 44 that are designed for connection to a primary concentrate container 2. Both the first primary container access 43 and the second primary container access 44 are in fluid communication with the main fluid line 4 of the fluid circuit 14. If the primary concentrate container 2 has just one inlet/outlet, for example because it contains a liquid concentrate, exclusively one of the two accesses 43, 44 is used (see FIGS. 4 and 4A). If, vice versa, the primary concentrate container 2 has one inlet and one outlet, both the first primary container access 43 and the second primary container access 44 are used (see e.g., FIGS. 1-3 and 4B/4C).

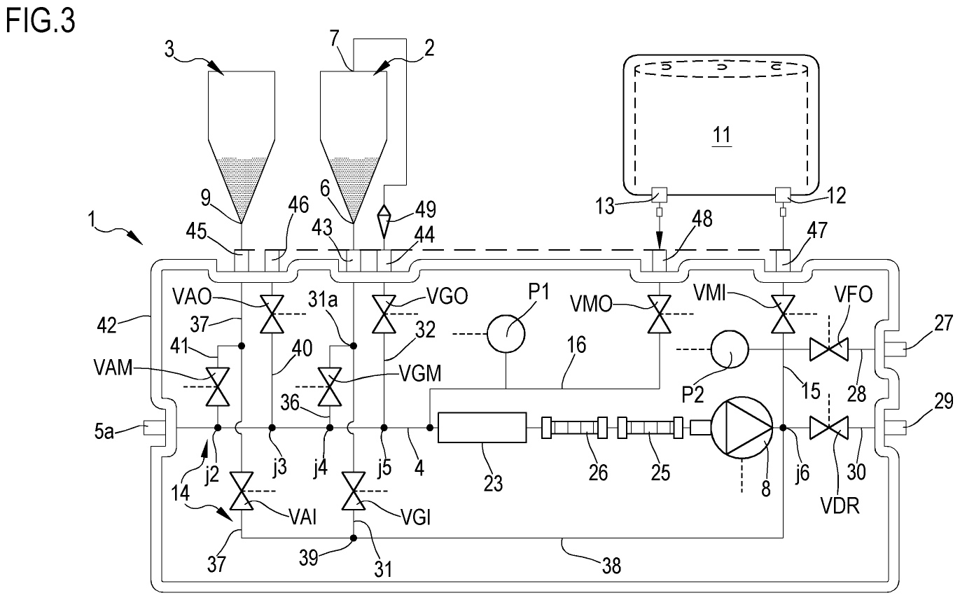

The supporting structure 42 also has a first auxiliary container access 45 and a second auxiliary container access 46 that are designed for connection to an auxiliary concentrate container 3. Both the first auxiliary container access 45 and the second auxiliary container access 46 are in fluid communication with the main fluid line 4 of the fluid circuit 14. If the auxiliary concentrate container 3 has just one inlet/outlet, for example because it contains a liquid concentrate, exclusively one of the two accesses 45, 46 is used (see FIGS. 3-4 and 4A). If, vice versa, the auxiliary concentrate container 3 has one inlet and one outlet, both the first auxiliary container access 45 and the second auxiliary container access 46 are used (see e.g., FIGS. 1-2 and 4B/4C).

As mentioned, the medical fluid generation apparatus 1 is provided with at least one concentrate container and preferably two concentrate containers 2, 3 having each at least one port 6, 7, 9, 10 that may be or is put in fluid communication with the main fluid line 4 of the fluid circuit 14 via the corresponding container access 43, 44, 45, 46.

Preferably, the medical fluid generation apparatus 1 comprises a primary concentrate container 2 having at least one port 6, 7 that may be or is put in fluid communication with the main fluid line 4 of the fluid circuit 14 and an auxiliary concentrate container 3 having at least one port 9, 10 that may be or is put in fluid communication with the main fluid line 4 of the fluid circuit 14. Each of the primary concentrate container 2 and the auxiliary concentrate container 3 defines, at least partially, a corresponding external additional part or extension of the fluid circuit 14 which may be connected to the corresponding first primary container access 43 and the corresponding first auxiliary container access 45.

Considering the embodiments shown in FIGS. 1, 2 and 3 the primary concentrate container 2 has a first port 6 in fluid communication with the main fluid line 4 of the fluid circuit 14 through the corresponding first primary container access 43 and the second port 7 in fluid communication with the main fluid line 4 of the fluid circuit 14 through the corresponding second primary container access 44. Further, a filter 49 is present in the line connecting the second port 7 to the second primary container access 44 to prevent foreign elements (e.g., granulate) from reaching internal components of the apparatus.

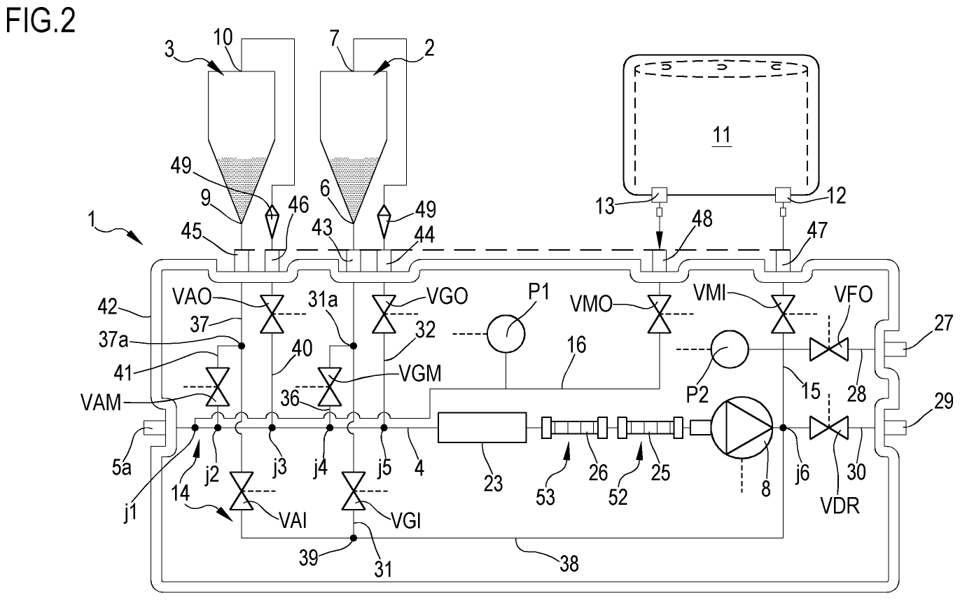

With reference to the embodiments shown in FIGS. 4 and 4A, the primary concentrate container 2 has only one port 6, the first one, in fluid communication with the main fluid line 4 of the fluid circuit 14, preferably through the first primary container access 43. Generally, when the primary concentrate container 2 has only the first port 6 to be connected to the first primary container access 43, it houses a primary liquid concentrate LC″ ready to be mixed with at least another liquid (such as water) or another liquid concentrate still ready to be mixed or obtained through a corresponding dissolution procedure. Considering the embodiments shown in FIGS. 1 and 2 the auxiliary concentrate container 3 has a first port 9 in fluid communication with the main fluid line 4 through the first auxiliary container access 45 of the fluid circuit 14 and a second port 10 in fluid communication with the main fluid line 4 of the fluid circuit 14 through the second auxiliary container access 46. Further, a filter 49 is present in the line connecting the second port 10 to the second auxiliary container access 46 to prevent foreign elements (e.g., granulate) from reaching internal components of the apparatus.

Referring to the embodiments shown in FIGS. 3 and 4, the auxiliary concentrate container 3 has only one port 9, the first one, in fluid communication with the main fluid line 4 of the fluid circuit 14 preferably through the first auxiliary container access 45. Generally, when the auxiliary concentrate container 3 has only the first port 9 to be connected to the first auxiliary container access 45, it houses an auxiliary liquid concentrate LC′ ready to be mixed with at least another liquid or liquid concentrate still ready to be mixed or obtained through a corresponding dissolution procedure.

Of course, the supporting structure 42 may present exclusively three accesses 43, 44 and 45 in case only one, two or three accesses are required at maximum; for example one primary concentrate 2 may include two ports 6, 7 that have to be connected to the supporting structure 42, thereby requiring two respective accesses e.g., 43, 44, and one auxiliary container 3 may include just one port 9 to be connected to e.g., access 45. In this case, access 46 of FIG. 3 may be absent. Correspondingly, the supporting structure 42 may have only two accesses 45, 46 in case both primary and auxiliary concentrates 2, 3 have one respective port 6, 9 to be connected to the respective access 43, 45 (see FIG. 4A—accesses 44 and 46 are not provided/necessary in this embodiment).

Regardless the specific embodiment to which it refers, the ports 6, 7, 9, 10 of the respective concentrate containers 2, 3 may be or are put in fluid communication with the main fluid line 4 of the fluid circuit 14 via the corresponding container accesses 43, 44, 45, 46 provided on the supporting structure 42. Therefore, the container accesses 43, 44, 45, 46 of the supporting structure 42 allow the medical generation apparatus 1 to provide different configurations of the concentrate containers 2, 3 to be used, which may house dry concentrates DC to be dissolved and/or diluted and/or liquid concentrates LC′; LC″ to be diluted. In particular, it has to be noted that in the embodiments shown in FIGS. 1, 2, 4B, and 4C, the auxiliary concentrate container 3 houses a dry concentrate DC to be dissolved and/or diluted, whereas in the embodiments shown in FIGS. 3, 4, and 4A, the auxiliary concentrate container 3 houses an auxiliary liquid concentrate LC′ to be diluted and mixed with another liquid concentrate.

With reference to FIGS. 1, 2, 3, 4, 4A, 4B, and 4C the medical fluid generation apparatus 1 has a mixing container 11 having an inlet 12 and an outlet 13 in fluid communication with the main fluid line 4 of the fluid circuit 14. According to the embodiment shown in FIG. 1, the mixing container 11 is operatively housed inside the supporting structure 42 without being easily accessible from the outside unless the supporting structure 42 is appropriately open. When the mixing container 11 is operatively placed inside the supporting structure 42, as shown in FIG. 1, the medical fluid generation apparatus 1 is provided with a sensor 17 for producing and giving information relating to the volume of the fluid or the fluids contained in the mixing container 11 itself.

The sensor 17 comprises at least one weight scale 17a and/or at least one level sensor 17b to produce an output signal relating to a significant volume value data inside the mixing container 11. Sensors different to the ones just mentioned may be alternatively used in order to detect the volume/level of fluid inside the mixing container 11. For example, the mixing container 11 may be filled with the vent 22 closed and the head space is determined from a pressure increase inside the container (at least initial pressure, final pressure, and pumped volume should be known). Preferably, the mixing container 11 comprises a substantially closed container (apart from the inlet and outlet for liquid and the vent).

When the mixing container 11 is operatively placed inside the supporting structure 42 as shown FIG. 1, it is preferred that the medical fluid generation apparatus 1 is provided with at least one sterilization device 18 configured to operate on the fluid volume contained inside the mixing container 11. In particular, the sterilization device 18 comprises a UV sterilization device which is placed inside the mixing container 11, for example located on a bottom wall 11a of this latter. Generally, the mixing container 11 which is operatively placed inside the supporting structure 42, is a non-removable and/or non-disposable container.

Additional (or alternative) sterilization devices may also be used. For example, one or more sterilization devices (e.g., UV devices) may be placed on other portions of the fluid circuit 14, such as the main fluid line 4.

According to the embodiment shown in FIG. 1, the mixing container comprises a vent 19 which includes a vent line 20 to selectively expel excess air contained inside. Preferably, the vent 19 is placed on a top portion 11b of the mixing container 11, more preferably the vent 19 is placed on a highest upper wall of the mixing container 11. Optionally, the vent 19 is provided with a filter 21 which is able to filter any air passing through the vent line 20 and/or an air valve 22 to selectively block air passage through the vent line 20. The valve 22 of the vent 19 is placed along the vent line 20, in particular between the mixing container 11 and the filter 21. Moreover, at an end of the vent line 20, downstream the filter 21 and the air valve 22, according to a position opposite the mixing container 11, the vent line 20, and therefore the vent 19, are connected to an air drain 1a of the medical generation apparatus 1. Note that the air drain 1a may open to external environment or may be connected to a drain outlet line 30 of the apparatus, wherein fluid to be discharged is conveyed and drained (as below explained in more detail).

Referring to the embodiments shown in FIGS. 2, 3 and 4, the mixing container 11 is positioned outside the support structure 42, being both visible and directly accessible from the outside. Advantageously, when the mixing container 11 is placed outside the supporting structure 42, the mixing container 11 may comprise a bag, preferably flexible, particularly transparent. Since the mixing container 11 is a transparent flexible bag, the volume of fluid contained therein may be immediately verified through the transparent structure of the flexible bag.

A sensor 17 able to verify the volume of fluid inside the mixing container 11 when this latter is placed inside the supporting structure 42 may be also used when such a mixing container 11 is operatively and/or physically placed outside the supporting structure 42.

It is preferred that when the mixing container 11 is operatively and/or physically placed outside the supporting structure 42 it is a removable and/or a disposable container. In order to be removable from the supporting structure 42, according to the embodiments shown in FIGS. 2, 3 and 4, the supporting structure 42 has a mixing container access port 47 of the fluid circuit 14 and a mixing container exit port 48 of the fluid circuit 14 for fluid communication with the inlet 12 of the mixing container 11 and the outlet 13 of the mixing container 11 respectively.

In particular, the mixing container access port 47 and the mixing container exit port 48 are both in fluid communication with the main fluid line 4 of the fluid circuit 14, to put this latter in fluid communication with the inlet 12 and the outlet 13 of the mixing container 11. Advantageously, both the inlet 12 and the outlet 13 of the mixing container 11 include self-sealing valves to prevent fluid from exiting the mixing container 11 once separated from the medical fluid generation apparatus 1 when an exchange of the used mixing container 11 needs to be performed with another one mixing container 11 not in use.

Detailing more the medical fluid generation apparatus 1 according to FIG. 1, the main fluid line 4 develops from the inlet point 5 towards the pump 8 reaching, in order, a first junction point j1, a second junction point j2, a third junction point j3, a fourth junction point j4, a fifth junction point j5, a heater 23 configured to heat the fluid circulating in the medical fluid generation apparatus 1 and at least one sensor 52 and particularly two sensors 52, 53 placed in series with each other. Either one or both sensors 52, 53 are configured to measure a property of the fluid flowing in the main fluid line 4. In general, the sensor/s 52, 53 may measure any one of a conductivity of the fluid, a concentration of a substance in the fluid, a concentration dependent physical properties, such as sound velocity, viscosity, density, a temperature of the fluid, and an optical property of the fluid flowing in the main fluid line 4 (and combinations thereof). For example, sensor 52 and/or sensor 53 may be a conductivity sensor, a concentration sensor, a temperature sensor, an optical sensor, or a sound sensor.

In a specific embodiment, sensor 52 is a conductivity meter 25, and sensor 53 is a conductivity meter 26, too; preferably the two conductivity meters 25, 26 are positioned in series with each other.

The conductivity meter 25 is configured to determine a conductivity of the fluid or the fluids circulating in the medical fluid generation apparatus 1. The second conductivity meter 26 is configured to independently determine the conductivity of the fluid or the fluids circulating in the medical fluid generation apparatus 1. Both the conductivity meter 25 and the second conductivity meter 26 determine and detect the conductivity of the same fluid flowing along the main fluid line 4 to provide a back-up measure.

A temperature sensor T1 is also present in the main fluid line 4; usually, but not necessarily, the temperature sensor T1 is incorporated in the conductivity meter 25 and provides a temperature signal to the control unit 24. The temperature signal is used to control the heater 23 and the heating power. Further, the temperature signal is also used to check that the conductivity meter 25 is working in its optimal temperature range. Since two conductivity meters are provided 25, 26, two temperature sensors T1 and T2 are also provided. The T sensors are shown in FIGS. 1 and 4A only. Of course, temperature sensor/s T1 and/or T2 may be sensor/s separated from other components (i.e., independent components measuring the temperature of the fluid flowing in the main fluid line 4 at different locations, usually downstream the fifth junction point j5 and upstream the sixth junction point j6.

The heater 23 and the conductivity meters 25, 26 (and the temperature sensors T1 and/or T2) are controlled by the control unit 24 which is properly set for managing the signals and data sent to and received from the heater 23 and the conductivity meters 25, 26.

Each of the elements just mentioned, among the first five junction points j1, j2, j3, j4, j5, the heater 23 and the conductivity meters 25, 26 will be described in detail throughout this description.

As it is possible to see in FIG. 1, the first junction point j1, the second junction point j2, the third junction point j3, the fourth junction point j4 and the fifth junction point j5 are positioned in series one after the other from the inlet point 5 to the pump 8, i.e. the first junction point j1 is downstream the inlet point 5, the second junction point j2 is downstream the first one, the third junction point j3 is downstream the second one, the fourth junction element j4 is downstream the third one, the fifth junction element j5 is downstream the fourth one.

The heater 23 is positioned downstream the fifth junction point j5 and the conductivity meters 25, 26 are located downstream the heater 23 and upstream the pump 8.

Therefore, the first junction point j1, the second junction point j2, the third junction point j3, the fourth junction point j4 the fifth junction point j5, the heater 23 and the conductivity meters 25, 26 are upstream the pump 8.

The conductivity meter 25 is interposed between the heater 23 and the pump 8, and the second conductivity meter 26 is interposed between the heater 23 and the first conductivity meter 25. Of course, the placement of the conductivity meters 25 and 26 on the main fluid line 4 may be reversed. Further, the heater 23 is generally placed upstream the conductivity meter/s 25, 26 for improved temperature control (e.g., to 37 °C) at the site of the conductivity meters 25, 26. A different configuration with the heater 23 placed downstream one or both the conductivity meter/s 25, 26 may work since, as below explained, a closed recirculation loop is used to mix and homogenize fluids.

Unlike the elements just mentioned which are placed upstream the pump 8, a sixth junction element j6, provided on the main fluid line 4, is positioned downstream the pump 8. Each junction point j1, j2, j3, j4, j5, j6 provides a connection between the main fluid line 4 of the fluid circuit 14 and at least one line, duct or branch of the fluid circuit 14 different to the main fluid line 4.

According to the embodiments shown in FIGS. 3 and 4, the position of the second junction point j2, the third junction point j3, the fourth junction point j4 and the fifth junction point j5, as well as the position of the heater 23, the conductivity meters 25, 26 and the sixth junction point j6 are the same as for the embodiment shown in FIG. 1. However, the position of the first junction point j1 changes. In particular, according to the embodiments shown in FIGS. 3 and 4, the first junction point j1 is placed on the main fluid line 4 between the fifth junction point j5 and the heater 23.

Therefore, according to the embodiments shown in FIGS. 3 and 4, the second junction point j2 is placed upstream the third one, the third junction point j3 is placed upstream the fourth one, the fourth junction point j4 is placed upstream the fifth one, the fifth junction point j5 is placed upstream the first one, the first junction point j1 is placed upstream the heater 23, which is placed upstream the conductivity meters 25, 26 which are placed upstream the pump 8, which is placed upstream the sixth junction point j6. This is a less preferable, but working, option.

The position of the first junction point j1 may change on the main fluid line 4 of the fluid circuit 14 with respect to the first four junction points j2, j3, j4, j5, but it cannot change with respect to the heater 23, the conductivity meters 25, 26 and the sixth junction point j6, in the sense that the first junction point j1 must be placed upstream the sixth junction point j6. The described relative position of junction points, heater, and meters is relevant for the proper working of the apparatus during recirculation and mixing. Indeed, the temperature of the recirculated fluid needs to be controlled by the heater 23 and its conductivity needs to be measured during the recirculation. Consequently, as apparent from the following description of the process for dissolving concentrates and/or mixing fluids, the heater 23 and the conductivity meters 25, 26 have to be included in a recirculation path R1. Furthermore, the position of the first junction point j1 is normally upstream all the other junction points j2, j3, j4, j5 so that any fluid coming from mixing chamber 11 may flush all tracts of the main fluid line 4 where a fluid injection is provided. However such positioning is only recommended, but not strictly necessary.

Going back to the embodiment shown in FIG. 1, the plurality of valves comprises a water inlet valve VWI placed on the main fluid line 4 between the inlet point 5 and the first junction point j1. When the valve VWI is open, the fluid arriving from the entrance 5a of the supporting structure 42 and the inlet point 5 of the main fluid line 4 of the fluid circuit 14 may flow along the main fluid line 4 towards the first five junction points j1, j2, j3, j4, j5, the heater 23, the conductivity meters, 25, 26 the pump 8 and the sixth junction point J6. When the valve VWI is closed, the fluid passage is blocked upstream the first junction point j1.

According to the embodiments shown in FIGS. 2, 3 and 4, the inlet point 5 may also be directly connected to the main fluid line 4. In this case, the main fluid line 4 is always fed by the entrance 5a and the inlet point 5.

Going on with the detailed description of the fluid circuit 14 common to all the embodiments, the fluid circuit 14 comprises a mixing container inlet line 15 connecting the sixth junction point j6 placed on the main fluid line 4 to the inlet 12 of the mixing container 11.

The plurality of valves comprises a mixing container inlet valve VMI configured to operate on the mixing container inlet line 15. The sixth junction j6 is operatively interposed between the pump 8 and the container inlet valve VMI. The mixing container inlet line 15 is directly connected to the inlet 12 of the mixing container 11, whereas with reference to the embodiments shown in FIGS. 2, 3 and 4, the mixing container inlet line 15 is connected to the inlet 12 of the mixing container 11 through the mixing container access port 47.

Advantageously, the medical fluid generation apparatus 1 comprises a pressure sensor P2 configured for sensing a pressure occurring on the main fluid line 4 downstream the pump 8. The pressure sensor P2 may be placed at any location of the fluid circuit experiencing the same pressure that is present in the main fluid line 4 downstream the pump. In the described embodiment, the pressure sensor P2 is placed on the mixing container inlet line 15, optionally between the sixth junction point j6 and the mixing container inlet valve VMI.

Referring to FIGS. 1, 2, 3, 4, 4A, 4B and 4C, the fluid circuit 14 further comprises a medical fluid outlet 27 to feed an external medical apparatus (not shown in any figure) like, for example, a dialysis apparatus, such as a peritoneal cycler or a hemodialysis apparatus, with the fluid arriving from the sixth junction point j6. In particular, the fluid outlet 27 is connected to the main fluid line 4 via a medical fluid outlet line 28 which is directly connected to the mixing container inlet line 15, downstream the sixth junction point j6 and the pump 8, and upstream the mixing container inlet valve VMI. The fluid flowing along the main fluid line 4 runs from the mixing container inlet line 15 towards the mixing container inlet valve VMI and the medical fluid outlet line 28.

Along the medical fluid outlet line 28, the plurality of valves comprises a medical fluid outlet valve VFO configured to operate on such medical fluid outlet line 28 to block the fluid upstream the fluid outlet 27 or allow such fluid to get out of the medical generation apparatus 1 towards the external medical apparatus via the fluid outlet 27.

The fluid circuit 14 further comprises a drain port 29 to discard an amount of fluid arriving from the sixth junction j6. In particular, the drain port 29 is connected to the main fluid line 4 through a drain outlet line 30 and the sixth junction point j6. In this case, the plurality of valves further comprises a drain valve VDR configured to operate on the drain outlet line 30 to block the fluid coming from the main fluid line 4 and the sixth junction point j6 upstream the drain port 29 or allow such a fluid to get out the medical generation apparatus 1 via the drain port 29.

In all the embodiments, the fluid circuit 14 comprises a mixing container outlet line 16 connecting the outlet 13 of the mixing container 11 to the first junction point j1 on the main fluid line 4. The plurality of valves comprises a mixing container outlet valve VMO configured to operate on the mixing container outlet line 16 to block the fluid coming from the mixing container 11 upstream the first junction point j1 or allow such a fluid to flow to this latter and the main fluid line 4.

The medical fluid generation apparatus 1 further comprises an auxiliary pressure sensor P1 placed on the mixing container outlet line 16, preferably between the first junction element j1 and the mixing container outlet valve VMO.

According to the embodiment shown in FIG. 1, the mixing container outlet line 16 is directly connected to the outlet 13 of the mixing container 11, whereas referring to the embodiments shown in FIGS. 2, 3 and 4, the mixing container outlet line 16 is connected to the outlet 13 of the mixing container 11 through the mixing container exit port 48.

According to the embodiments shown in FIGS. 2, 3 and 4, the fluid enters and exits from the mixing container 11 respectively through the mixing container access port 47 and the mixing container exit port 48.

As previously mentioned, in addition or alternatively to described embodiments, one or more sterilization devices (e.g., UV devices) may be placed at the mixing container inlet line 15 and/or the mixing container outlet line 16.

In all the embodiments, at least one mixing recirculation path R1 (marked up in FIG. 1P) is provided. In this case, the plurality of valves and the control unit 24 are configured to allow a recirculation of at least one fluid contained in the mixing container 11 through the outlet 13, at least one portion of the main fluid line 4 on which the pump 8 is configured to operate, and the inlet 12. In particular, the mixing recirculation path R1 comprises the mixing container 11, the outlet 13, the mixing container outlet line 16 connecting the outlet 13 of the mixing container 11 to the first junction point j1 on the main fluid line 4, at least one portion of the main fluid line 4 on which the pump 8 is configured to operate from the first junction point j1 to the sixth junction point j6, the mixing container inlet line 15 connecting the sixth junction point j6 on the main fluid line 4 to the inlet 12 of the mixing container 11, and the inlet 12. More specifically, the mixing recirculation path R1 comprises the mixing container outlet valve VMO configured to operate on the mixing container outlet line 16 and the mixing container inlet valve VMI configured to operate on the mixing container inlet line 15. In order to set the mixing recirculation path R1, the mixing container outlet valve VMO and the mixing container inlet valve VMI are maintained open to let any fluid recirculating in the mixing recirculation path R1, while all of the other valves of the plurality of valves of the medical generation apparatus 1 configured to operate on the fluid circuit 14, which are different to the mixing container outlet valve VMO and the mixing container inlet valve VMI, are maintained close.

In all the embodiments, the fluid circuit 14 comprises a primary concentrate container inlet line 31 connecting the sixth junction point j6 on the main fluid line 4 to the first primary container access 43 of the supporting structure 42. The first primary container access 43 may be connected to the first port 6 of the primary concentrate container 2 as shown in the enclosed embodiments. As explained in the following, first port 6 may be used as inlet to feed fluid to the primary concentrate container 2 or as an outlet to remove fluid from the same.

The plurality of valves comprises a primary concentrate container inlet valve VGI configured to operate on the primary concentrate container inlet line 31, particularly placed between the first primary container access 43 of the supporting structure 42 and the sixth junction point j6. The fluid circuit 14 comprises a primary concentrate container outlet line 32 connecting the second primary container access 44 of the supporting structure 42 to the fifth junction point j5 on the main fluid line 4. The second primary container access 44 of the supporting structure 42 may be connected to the second port 7 of the primary concentrate container 2 as shown in the embodiments of FIGS. 1, 2, 3, 4B, and 4C.

In all embodiments, but the simplified embodiment of FIG. 4A, the valves of the plurality of valves comprise a primary concentrate container outlet valve VGO configured to operate on the primary concentrate container outlet line 32, preferably between the second primary container access 44 and the fifth junction point j5.

Considering particularly the embodiment shown in FIG. 1, the medical fluid generation apparatus 1 further comprises a concentration sensor SG configured for sensing a concentration of at least one substance to be detected in the fluid flowing towards the first primary container access 43, especially when this latter is connected to the first port 6 of the primary concentrate container 2. More in particular, the concentration sensor SG is preferably placed on the primary concentrate container inlet line 31, optionally between the primary concentrate container inlet valve VGI and the first primary container access 43. Although the concentration sensor SG is only shown with reference to the embodiment of FIG. 1, such kind of concentrate sensors may be applied in other parts or lines of the fluid circuit 14 in order to check the concentration of at least one substance to be detected in the fluid running along the fluid circuit 14 and, in particular towards the concentrate containers 2, 3. Moreover, any concentration sensor SG provided on the fluid circuit 14 according to the embodiment shown in FIG. 1, may also be provided on the fluid circuit 14 according to the embodiments shown in FIGS. 2, 3, 4, 4A, 4B, and 4C. The concentration sensor SG may be a glucose concentration sensor.

The medical fluid apparatus 1 may further comprise at least one de-aeration chamber 33. One or more de-aeration chambers may be useful for procedures requiring to expel air from the fluid circuit 14 or the containers 2, 3, for example in case a container includes dry concentrate to be dissolved. Of course, in case only liquid concentrates are used, any de-aeration chamber may be optional. This will be apparent from the description of the implemented procedures described below. According to the embodiment of FIG. 1, the de-aeration chamber 33 is placed on the primary concentrate container outlet line 32, optionally between the second primary container access 44 of the supporting structure 42 and the primary concentrate container outlet valve VGO. The de-aeration chamber 33 separates the primary concentrate container outlet line 32 into a first portion 32a extending between the second primary container access 44 and an inlet to the de-aeration chamber 33 and a second portion 32b extending between an outlet of the de-aeration chamber 33 and the fifth junction point j5. In particular, the de-aeration chamber 33 comprises a vent 34 including a vent line 35 to selectively expel excess air inside such de-aeration chamber 33. Optionally, a gas valve VGA is placed on the vent line 35 to selectively block air passage through such line. In particular, the gas valve VGA is placed on the vent line 35 according to a position interposed between the primary concentrate container 2 and a drain 1a and, more in particularly, between the de-aeration chamber 33 and the drain 1a.

Furthermore, the medical fluid generation apparatus 1 according to the embodiment shown in FIG. 1, comprises a level sensor associated with the de-aeration chamber 33 to check a fluid level inside this latter. Advantageously, the drain 1a for draining the excess air arriving from the de-aeration chamber 33 may be the same drain 1a used for expelling the excess air from the mixing container 11. Also in this case, the de-aeration chamber 33, the vent 34, the vent line 35 and the gas valve VGA, shown only with reference to the embodiment of FIG. 1, may be applied to the primary concentrate container outlet line 32 of the fluid circuit 14 of the medical generation apparatus 1 according to the embodiments shown in FIGS. 2, 3, 4, 4B, and 4C.

The fluid circuit 14 comprises a first concentrate mixing line 36 connecting the fourth junction point j4 placed on the main fluid line 4 to the first primary access port 43, particularly through an intermediate point 31a of the primary concentrate container inlet line 31, which is preferably placed proximate to the first primary container access 43, optionally upstream the concentrate sensor SG. In this case, the plurality of valves comprises a first concentrate mixing valve VGM configured to operate on the first concentrate mixing line 36. The fluid circuit 14 comprises an auxiliary concentrate container inlet line 37 connecting the sixth junction point j6 on the main fluid line 4 to the first auxiliary container access 45. The first auxiliary container access 45 may be connected to the first port 9 of the auxiliary concentrate container 3 as shown in the embodiments.

In all the embodiments, the plurality of valves comprises a second concentrate container inlet valve VAI configured to operate on the auxiliary concentrate container inlet line 37, particularly placed between the first auxiliary container access 45 of the supporting structure 42 and the sixth junction point j6.

The fluid circuit 14 comprises a second concentrate container outlet line 40 connecting the second auxiliary container access 46 of the supporting structure 42 to the third junction point j3 on the main fluid line 4. The second auxiliary container access 46 of the supporting structure 42 may be connected to the second port 10 of the auxiliary concentrate container 3 as shown in the embodiments of FIGS. 1, 2, 4B, and 4C.

The difference between the embodiment of FIG. 1 and the embodiment of FIGS. 4B and 4C is in the outline of the second concentrate outlet line 40. Indeed, the configuration of FIGS. 4B and 4C further provides a deaeration chamber placed along the second concentrate outlet line 40. A more detailed description of the two variants is provided in the following description.

When the first auxiliary container access 45 is connected to the first port 9 of the auxiliary concentrate container 3 as shown in FIGS. 3, 4, and 4A, the second auxiliary container access 46 is not connected to any port 9, 10 of the auxiliary concentrate container 3 for which the auxiliary concentrate container outlet line 40 becomes a sort of a dead-end line, not useful for supplying any fluid to the auxiliary concentrate container 3. This kind of connection is normally effected when the auxiliary concentrate container 3 houses a liquid concentrate or a liquid concentrate mixture ready to be used without requiring any dissolution procedure of a dry concentrate substance whereby the first port 9 is directly connected to the first auxiliary access 45 of the supporting structure 42.

According to the embodiments of FIGS. 1, 2, 3, 4, 4B and 4C, the plurality of valves further comprises a second concentrate container outlet valve VAO configured to operate on the second concentrate container outlet line 40, preferably between the second auxiliary container access 46 and the third junction point j3. The fluid circuit 14 comprises a second concentrate mixing line 41 connecting the second junction point j2 placed on the main fluid line 4 to the first auxiliary container access 45, particularly through an intermediate point 37a of the auxiliary concentrate container inlet line 37, which is preferably placed proximate to the first auxiliary container access 45. In this case, the valves of the plurality of valves comprise a second concentrate mixing valve VAM configured to operate on the second concentrate mixing line 41.

Still considering all the embodiments, but the embodiment of FIG. 4A, the auxiliary concentrate container inlet line 37 comprises a common tract 38 with the primary concentrate container inlet line 31. The common tract 38 develops between the sixth junction point j6 and a branch 39 where the primary concentrate container inlet line 31 and the auxiliary concentrate container inlet line 37 separate each from the other. In particular, the common tract 38 does not include any valve. The primary concentrate container inlet valve VGI is configured to operate on the primary concentrate container inlet line 31 downstream the branch 39 and the auxiliary concentrate container inlet valve VAI is configured to operate on the auxiliary concentrate container inlet line 37 downstream the branch 39.

To summarize, the main differences between the embodiment shown in FIG. 2 and the embodiment shown in FIG. 1, are: the absence, in FIG. 2, of any de-aeration chamber, any vent line and any gas valve; and, the provision of a disposable mixing container or bag 11 external to the supporting structure 42.

Concerning the difference just mentioned first, FIG. 2 is a simplified schematic view of the fluid medical generation apparatus 1 shown in FIG. 1. The apparatus 1 shown in FIG. 2 may be provided with a de-aeration chamber, a corresponding vent line and a corresponding gas valve for one or both concentrate outlet lines 32, 40. In this regard, FIG. 4B shows the fluid circuit 14 that includes both de-aeration chamber 33 and auxiliary de-aeration chamber 51 placed on the respective outlet lines 32 and 40. Both de-aeration chambers 33, 51 share a common tract of the vent line 35. A gas valve VGA selectively allows gas passage from the de-aeration chamber 33 towards the air drain 1a (expelling gas in the external environment or conveying the gas to the drain outlet line 30). The auxiliary gas valve VGA2 selectively allows gas passage from the auxiliary de-aeration chamber 51 towards the air drain 1a.

Alternatively, in order to allow the air inside the concentrate containers 2, 3 to drain out, it is possible to provide a common de-aeration chamber and/or a common vent line with a common gas valve connected to the concentrate outlet lines 32, 40 of both concentrate containers 2, 3. The embodiment is shown in FIG. 4C, wherein the auxiliary concentrate container outlet line 40 is directed to the first portion of the primary concentrate container outlet line 32a and thereafter to the de-aeration chamber 33. The auxiliary concentrate container outlet valve VAO selectively opens and closes the auxiliary concentrate container outlet line 40 depending on the operating condition. In the embodiment of FIG. 4C, the second portion 32b of the primary concentrate container outlet line 32 is in common between the outlet lines 32 and 40 from the primary and auxiliary concentrate containers 2, 3. The injection point into the main fluid line 4 is in common as well (fifth injection point j5). The third injection point j3 is not present in the embodiment of FIG. 4C.

Considering the embodiment shown in FIG. 3, only the primary concentrate container 2 may house a dry concentrate DC.

Also in this case, the embodiment shown in FIG. 3 lack providing the de-aeration chamber, the vent line and the gas valve shown in FIG. 1 with reference to the primary concentrate outlet line 32, necessary to allow the air inside the primary container 2 to drain out. However, as already explained in relation to the embodiment shown in FIG. 2, also FIG. 3 refers to a simplified version of the apparatus 1 shown in FIG. 1. The primary concentrate outlet line 32 according to the embodiment shown in FIG. 3 may be provided with a de-aeration chamber, an auxiliary de-aeration chamber, a vent line, and a gas valve, useful for draining the air contained in the primary container 2. In other terms any of the embodiments of FIG. 1, 4B, and 4C with regard to the de-aeration chamber/s, valve/s, and line/s may also be implemented in the embodiment of FIG. 3.

Additionally, in FIG. 3, the position of the first junction point j1 is between the fifth junction point j5 and the heater 23. This is to show that the first junction point j1 could be placed in different positions (in any of the disclosed embodiments), namely downstream the second junction point j2 and upstream the third junction point j3, downstream the third junction point j3 and upstream the fourth junction point j4, downstream the fourth junction point j4 and upstream the fifth junction point j5, or as shown in FIG. 3. Moreover, the different position of the first junction point j1 in the embodiment shown in FIG. 3 with respect to its position in the embodiment shown in FIG. 1 does not change the operation of the apparatus 1 because the first junction point j1 concerns the successive mixing operations without interfering with the dissolution and recirculation of the liquid mixtures ML obtained. Therefore, all the steps of the dry concentrate dissolution procedures explained in the following description by using FIGS. 1A to 1K will be the same for the embodiment shown in FIG. 3 as well.

According to FIGS. 1F, 1K which refer to the embodiment shown in FIG. 1, the control unit 24 is configured to interact with the pump 8 and the plurality of valves to set at least one concentrate recirculation path Z6, X5 wherein the valves of the plurality of valves are configured to allow a recirculation of the fluid contained and/or the fluid produced into in one of the primary concentrate container 2 and/or the auxiliary concentrate container 3 through: the first port 6, 9 at least one portion of the main fluid line 4 on which the pump 8 is configured to operate, and the second port 7, 10.

In particular, considering FIG. 1F, the concentrate recirculation path comprises a first concentrate recirculation path Z6 which includes the primary concentrate container 2, the second port 7, the second primary container access 44, the first portion 32a of the primary concentrate container outlet line 32, the de-aeration chamber 33, the second portion 32b of the primary concentrate outlet line 32, a fifth junction point j5 on the main fluid line 4, the corresponding portion of the main fluid line 4 on which the pump 8 is configured to operate from the fifth junction point j5 to a sixth junction point j6, the primary concentrate container inlet line 31 connecting the sixth junction point j6 on the main fluid line 4 to the first port 6 of the primary concentrate container 2 via the first primary container access 43, and the first port 6.

In this case, the primary concentrate container outlet valve VGO is configured to operate on second portion 32b of the primary concentrate container outlet line 32 and the primary concentrate container inlet valve VGI is configured to operate on the primary concentrate container inlet line 31. When the control unit 24 controls the plurality of valves to set the first concentrate recirculation path Z6, the primary concentrate container outlet valve VGO and the primary concentrate container inlet valve VGI are maintained open to let the fluid recirculating in the first concentrate recirculation path Z6, while the other valves of the plurality of valves configured to operate on the fluid circuit 14 are maintained closed to keep the fluid running along the first concentrate recirculation path Z6.

When the primary concentrate container 2, as in the embodiment shown in FIG. 4, houses a fluid ready to be mixed and/or used, such primary container 2 does not have the outlet port 7 and is properly connected, through the inlet port 6, directly to the first primary container access 43. In this case, the first concentrate recirculation path Z6 is never set because the fluid inside the primary concentrate container 2 may only flow out towards the main fluid line 4. When the first concentrate mixing valve VGM is open and the pump 8 is activated, the fluid placed inside the primary concentrate container 2 is sucked by the pump 8 to flow into the fluid circuit 14, running along the portion of the main fluid 4 extending between the fourth junction point j4 and the sixth junction point j6, to arrive in the mixing container inlet line 15.

Considering FIG. 1K, the concentrate recirculation path comprises a second concentrate recirculation path X5 which includes the auxiliary concentrate container 3, the second port 10, the second auxiliary container access 46, the auxiliary concentrate container outlet line 40, the third junction point j3 on the main fluid line 4, the corresponding portion of the main fluid line 4 on which the pump 8 is configured to operate from the third junction point j3 to the sixth junction point j6, the auxiliary concentrate container inlet line 37 connecting the sixth junction point j6 to the first port 9 of the auxiliary concentrate container 3 via the first auxiliary container access 45.

As shown in FIGS. 4B and 4C, the auxiliary concentrate container outlet line 40 may be provided with an auxiliary de-aeration chamber 51 (FIG. 4B) or connected to the de-aeration chamber 33 (FIG. 4C); a vent 34, a vent line 35a and an auxiliary gas valve VGA2 as the one configured to operate on the primary concentrate container outlet line 32 may be provided (FIG. 4B) or a line may be connected to the de-aeration chamber 33 of the primary concentrate container outlet line 32, by means of the connections shown in FIG. 4C.

The auxiliary concentrate container outlet valve VAO is configured to operate on the auxiliary concentrate container outlet line 40 and the auxiliary concentrate container inlet valve VAI is configured to operate on the auxiliary concentrate container inlet line 37. When the control unit (24) controls the plurality of valves to set the second concentrate recirculation path X5, the auxiliary concentrate container outlet valve VAO and the auxiliary concentrate container inlet valve VAI are maintained open to let the fluid recirculating in the second concentrate recirculation path X5, while the other valves of the plurality of valves configured to operate on the fluid circuit 14 are maintained closed to keep the fluid running along the second concentrate recirculation path X5.

When the auxiliary concentrate container 3, as in the embodiment shown in FIGS. 3, 4, and 4A, houses a fluid ready to be mixed and/or used, such auxiliary concentrate container 3 is not provided with the outlet port 10 and is properly connected, through the inlet port 9 directly to the first auxiliary container access 45. In this particular case, the second concentrate recirculation path X5 cannot be set because the fluid inside the auxiliary concentrate container 3 may only flow out towards the main fluid line 4. When the second concentrate mixing valve VAM is open and the pump 8 is activated, the fluid placed inside the auxiliary concentrate container 3 is sucked by the pump 8 to flow into the fluid circuit 14, running along the portion of the main fluid 4 extending between the second junction point j2 and the sixth junction point j6, to arrive in the mixing container inlet line 15.

When the concentrate containers 2, 3 house a concentrate substance, both in the form of a powder/granulate and in the form of a liquid, to be dissolved and/or diluted, the control unit 24 is configured and/or programmed to first set at least one recirculation path Z6, X5, preferably both, and subsequently to set the mixing recirculation path R1.

### Dry Concentrate Dissolution Procedure

Considering at least one of the concentrate containers 2, 3, in particular the primary concentrate container 2, housing a dry concentrate DC like a powder or a granulate, the control unit 24 is configured to run a dry concentrate dissolution procedure DCDP (FIG. 5) which includes sending a dissolution liquid DL, e.g., water, to such a concentrate container 2, 3 in order to dissolve the DC and form a concentrated mixture CM, which is a liquid or a liquid concentrate.

Advantageously, the DCDP includes heating the DL and/or the CM by the heater 23. Preferably, the DL is heated up to a temperature sufficient to achieve complete dissolution of the specific dry concentrate in a certain time period. In the following, the temperature values are provided for glucose for illustrative purposes only. For a different dry concentrate, for example for the buffer/electrolyte powder, some heating is expected to be required, too, but not at as high temperatures as for glucose. In other words, the disclosed temperatures were tested for glucose. For buffer concentrate the required temperatures are expected to be lower.

For glucose, the DL is heated up to at least 60 °C and more specifically up to at least 70 °C and the CM is heated up to at least 40 °C and more specifically up to at least 50 °C.

The DCDP includes at least one concentrate mixture recirculation step CMRS, CMRS-Aux in which the control unit 24 sets the corresponding concentrate recirculation path Z6, X5 and recirculates the CM to homogenize.

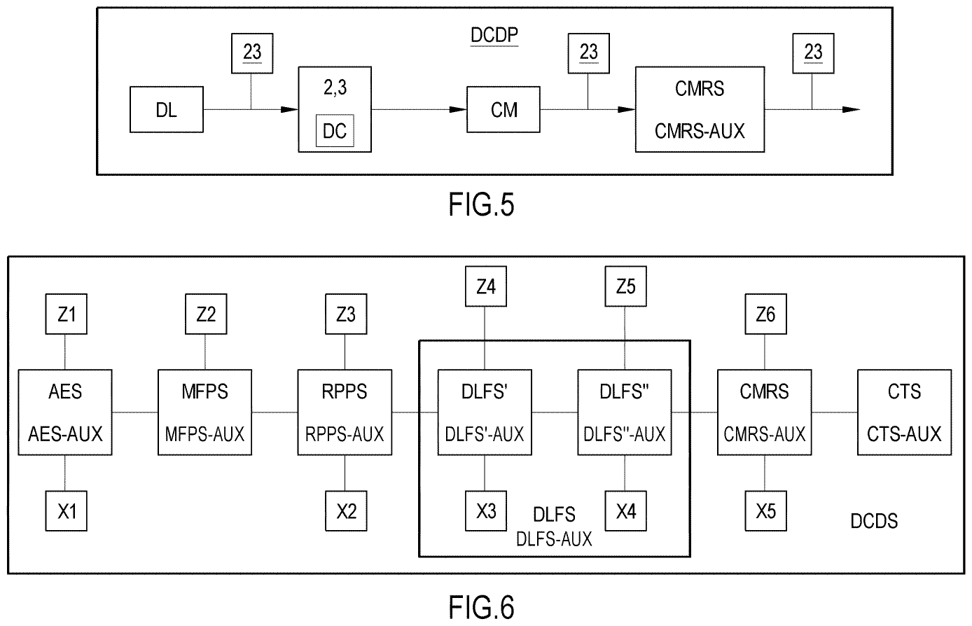

As schematically shown in FIG. 6, the DCDP includes an air evacuation step AES, a priming step of the main fluid line MFPS, a recirculation path priming step RPPS, a dissolution liquid fill step DLFS, a concentrate mixture recirculation step CMRS, and an optional concentration test step CTS. The DLFS may include two (or more) different steps, namely a first dissolution liquid fill step DLFS' and a second dissolution liquid fill step DLFS″. Optionally, in the DCDP, at least the air evacuation step AES, the dissolution liquid fill step DLFS (including the DLFS' and the DLFS″) and the concentrate mixture recirculation step CMRS, are executed by the control unit 24, preferably in time sequence.

The dry concentrate dissolution procedure will be described in more detail with reference to the figure sequence 1A to 1F.

The AES has the aim to evacuate air from the dry powder container (primary concentrate container 2 and/or auxiliary concentrate container 3) to fill the respective de-aeration chamber 33; 51 with air. This is performed to ensure that the air volume doesn't contribute to the water dead volume. The air evacuation fluid path Z1 is shown in FIG. 1A; the control unit 24 commands the plurality of valves to set the air evacuation flow path Z1 to evacuate air from the primary concentrate container 2 and commands the pump 8 to remove air from such primary concentrate container 2. In the air evacuation flow path Z1, the plurality of valves is configured to allow the air contained in the primary concentrate container 2 to flow into the primary concentrate container outlet line 32 and into a de-aeration chamber 33 when the pump 8 is active. The primary concentrate container outlet valve VGO acting on the primary concentrate container outlet line 32 and the drain valve VDR acting on the drain outlet line 30 are open, while all the other valves of the plurality of valves configured to operate on the fluid circuit 14 are closed. Advantageously, during the AES the control unit 24 operates the pump 8 at a pre-set pumping rate for a pre-fixed time period and stops the pump 8 after the pre-fixed time period has lapsed. During AES pumping is performed until low level in the deaeration chamber is ensured. In an illustrative embodiment for testing purposes, the pre-set pumping rate was set to about 300 ml/min and the pre-fixed time period was set to 20s.

Moving to FIG. 1B, the priming step of the main fluid line MFPS has the aim to prime the main fluid path (j5-j6) after the air evacuation; the control unit 24 commands the plurality of valves to set a main fluid line prime path Z2 and activates the pump 8 to prime the main fluid line 4, particularly after the AES. In particular, in the main fluid line prime path Z2 the valves of the plurality of valves are configured to allow a dissolution liquid DL, e.g., water, to enter into the main fluid line 4 and flow towards a drain outlet line 30 under the action of the pump 8. In this case, the water inlet valve VWI placed on the main fluid line 4 and the drain valve VDR acting on the drain outlet line 30 are open, while the other valves of the plurality of valves, different from the water inlet valve VWI and the drain valve VDR, are maintained closed.

During the MFPS the control unit 24 operates the pump 8 at a pre-set pumping rate up to a pre-fixed pumping volume and stops the pump 8 after the pre-fixed pumping volume has been reached. The pump 8 is operated until the pre-fixed pumping volume greater that the main fluid line volume from water inlet valve VWI to sixth junction point j6 is achieved. Optionally, a safety factor z (e.g. 1.5-2) is considered to the main fluid line volume to ensure that no air remains between the fourth junction point j4 and the sixth junction point j6. In the illustrative embodiment for testing purposes, the pre-set pumping rate was about 300 ml/min and the pre-fixed pumping volume was at least 100 ml.

Moving to FIG. 1C, the RPPS has the aim to prime the recirculation fluid path upstream the bag (namely the fluid path between sixth junction point j6 and fourth junction point j4) and direct any present air to the main fluid path (included between fourth junction point j4 and sixth junction point j6). The control unit 24 commands the valves of the plurality of valves to set a recirculation priming path Z3 and activates the pump 8 to prime the recirculation priming path Z3, particularly after the AES and the MFPS. Considering the recirculation priming path Z3, the valves of the plurality of valves are configured to allow fluid circulation from the sixth junction point j6 on the main fluid line 4 towards the primary concentrate container inlet line 31, into the first concentrate mixing line 36 to the fourth junction point j4 on the main fluid line 4, in at least a portion of the main fluid line 4 on which the pump 8 is active, to the sixth junction point j6.

In particular, the valves of the plurality of valves are configured to allow fluid circulation through the primary concentrate container inlet valve VGI acting on the primary concentrate container inlet line 31 and the first concentrate mixing valve VGM acting on the first concentrate mixing line 36 when such valves VGI, VGM are maintained open, while the other valves of the plurality of valves different from the primary concentrate container inlet valve VGI and the first concentrate mixing valve VGM and configured to operate on the fluid circuit 14, are maintained closed.

During the RPPS the control unit 24 operates the pump 8 at a pre-set pumping rate up to a pre-fixed pumping volume and stops the pump 8 after the pre-fixed pumping volume has been reached. The pre-fixed pumping volume to pump is lower than the main fluid line volume between fourth junction point j4 and sixth junction point j6, but also higher than the volume in the rest of the recirculation priming path Z3 (i.e., total volume of the recirculation priming path minus the main fluid line volume between fourth junction point j4 and sixth junction point j6). In the illustrative embodiment for testing purposes, the pre-set pumping rate was about 300 ml/min and the pre-fixed pumping volume was at least 100 ml.

The recirculation path priming step allows to relocate air that may be potentially housed in the lines to the main fluid line 4. After air relocation, the main fluid line is again primed by repeating the MFPS (see FIG. 1B). The purpose of this additional priming step is to re-prime the main fluid line from j5 to j6 after the air relocation. This step ensures that the water delivery fluid path to the concentrate container (j5-pump 8-VGI-j4) is primed so that its dead volume can be subtracted from the nominal water volume to add.

The DLFS' has the aim to fill a fraction of the expected total water volume and meanwhile establish and save a reference pressure value (hereafter called P2_base) indicative of a median-filtered pressure P2 value (average pressure) present in the line during the first dissolution liquid fill step. The reference pressure value (P2_base) will act as a base pressure value when further filling is performed with pressure feedback from sensor P2. Since gas valve VGA is open, air can freely pass out via the de-aeration chamber 33 as the fluid level rises in the primary concentrate container 2. The control unit 24 commands the valves of the plurality of valves to set a first dissolution liquid fill path Z4 (FIG. 1D) and commands the pump 8 to fill a fraction of an expected total dissolution liquid volume in the primary concentrate container 2, particularly after the AES, after the MFPS and after the RPPS. More specifically, in the first dissolution liquid fill path Z4, the valves of the plurality of valves are configured to allow dissolution liquid DL, e.g., water, to enter the primary concentrate container 2.

Preferably, the first dissolution liquid fill path Z4 comprises the main fluid line 4, the primary concentrate container inlet line 31, the first primary container access 43, the first port 6 of the primary concentrate container 2, the primary concentrate container 2, the second port 7 of the primary concentrate container 2, the first portion 32a of the primary concentrate container outlet line 32, the de-aeration chamber 33 and the vent 34 of the de-aeration chamber. In order to set the first dissolution liquid fill path Z4, the water inlet valve VWI placed on the main fluid line 4, the primary concentrate container inlet valve VGI on the primary concentrate container inlet line 31 and the gas valve VGA on the vent 34 are maintained open, while the other valves of the plurality of valves configured to operate on the fluid circuit 14 and different from the water inlet valve VWI, the primary concentrate container inlet valve VGI, and the gas valve VGA, are maintained closed.

During the DLFS' the control unit 24 operates the pump 8 at a pre-set pumping rate and ends the first dissolution liquid fill step after a liquid level in a de-aeration chamber 33 reaches a pre-set liquid high level. The purpose is to maintain the water filling until all air in the primary concentrate container 2 has been pushed out of the bag and the de-aeration chamber 33. Notably, the control unit 24 is configured to receive pressure data of a pressure in the first dissolution liquid fill path Z4 and to determine the reference pressure value P2_base indicative of the average pressure during at least a part of the first dissolution liquid fill step. Pressure is read through pressure sensor P2 on the mixing container inlet line experiencing the same pressure existing in the first dissolution liquid fill path Z4. Advantageously, the pressure data and/or the reference pressure value P2_base are stored in a memory associated with the control unit 24; as mentioned the reference pressure value P2_base is the reference pressure then used for further filling of the concentrate container.

During the DLFS' step the control unit 24 operates the heater 23 on the main fluid line 4 to heat the dissolution liquid up to a temperature sufficient to support efficient dissolution in later steps. In the illustrative embodiment for testing purposes, temperature was at least 70 °C and optionally up to at least 80 °C.

Moving to FIG. 1E, the DLFS″ has the purpose to maintain the water filling until a certain overpressure has been built up in the primary concentrate container 2. This ensures that the top outlet site does not collapse during recirculation and enables passive de-aeration during the recirculation. The control unit 24 commands the plurality of valves to set a second dissolution liquid fill path Z5 and commands the pump 8 to fill up to an expected total dissolution liquid volume in the primary concentrate container 2, particularly after the DLFS′. In the second dissolution liquid fill path Z5 the valves of the plurality of valves are configured to allow dissolution liquid DL, i.e., water arriving from the inlet point 5, to enter the primary concentrate container 2.

In particular, the second dissolution liquid fill path Z5 comprises the main fluid line 4, the sixth junction point j6, the primary concentrate container inlet line 31, the first primary container access 43, the first port 6 of the primary concentrate container 2, the primary concentrate container 2, the second port 7 of the primary concentrate container 2, the second primary container access 44, the first portion 32a of the concentrate container outlet line 32, the de-aeration chamber 33 and a part of the second portion 32b of the concentrate container outlet line 32 upstream the primary concentrate container outlet valve VGO. To set the second dissolution liquid fill path Z5, the water inlet valve VWI placed on the main fluid line 4 and the primary concentrate container inlet valve VGI placed on the primary concentrate container inlet line 31 are open, while the other valves of the plurality of valves different from the water inlet valve VWI and the primary concentrate container inlet valve VGI are closed.

In particular, during the DLFS″ the control unit 24 operates the pump 8 at a pre-set pumping rate up to a pre-fixed pressure in the second dissolution liquid fill path Z5 and stops the pump 8 after the pre-fixed pressure has been reached (the pre-fixed pressure is in particular the reference pressure value (P2_base), in the second dissolution liquid fill path Z5 plus an overpressure value); in the illustrative embodiment for testing purposes, the pre-set pumping rate was about 300 ml/min and the overpressure value was 40 mmHg.

Furthermore, during the DLFS″ the control unit 24 operates a heater 23 on the main fluid line 4 to heat the dissolution liquid up to at least 70 °C and optionally up to at least 80 °C.

In the step illustrated in FIG. 1F, the CMRS has the aim to completely dissolve the dry (e.g., glucose) powder and mix the concentrate to a uniform composition. In the recirculation process, functionality exists to de-aerate the de-aeration chamber 33 from accumulated air. If a low liquid level is detected in the de-aeration chamber 33, the recirculation is stopped and the gas valve VGA is opened to let air be pushed out by the over pressure in the primary concentrate container 2 until high liquid level is detected in the de-aeration chamber 33. The control unit 24 commands the plurality of valves to set a concentrate recirculation path Z6 and commands the pump 8 to re-circulate the CM to dissolve the DC and homogenously mix the concentrated mixture CM.

The CMRS is particularly performed after the DLFS' and after the DLFS″. During the CMRS the control unit 24 operates the pump 8 at a pre-set pumping rate for a pre-fixed time period and stops the pump 8 after the pre-fixed time period has lapsed. Furthermore, during the CMRS the control unit 24 operates a heater 23 on the main fluid line 4 to heat the concentrated mixture CM. The combination of time, flow rate, and temperature is chosen to effectively dissolve the powder and homogenize the solution. For example in the illustrative embodiment for testing purposes, the pre-set pumping rate was about 300 ml/min, the pre-fixed time period was 600s and the temperature was set up to at least 50 °C and optionally up to at least 60 °C.

Advantageously, the control unit 24 is configured to perform an optional concentration test step CTS to measure a concentration of the CM and to compare the measured concentration with an expected concentration of the concentrated mixture CM. The control unit 24 is configured to measure the concentration during the CMRS. The purpose is to test the concentration of the (glucose) concentrate by comparing its conductivity-decreasing effect with expected conductivity when a well-defined glucose concentrate volume is added to a well-defined electrolyte plus water solution.

### Dry Concentrate Dissolution Procedure Starting from the Auxiliary Container

In the place of starting from the primary concentrate container 2, the DCDP may begin from the auxiliary concentrate container 3 housing a dry concentrate like a powder or a granulate.

In this case, the control unit 24 is configured to run a dry concentrate dissolution procedure DCDP by determining a dissolution liquid DL, e.g., water, be sent to the auxiliary concentrate container 3 to dissolve the corresponding dry concentrate DC and form a corresponding CM, which is liquid.

Also in this specific case, the DCDP includes heating the DL and/or the CM by the heater 23. Preferably, the DL is heated up to at least 60 °C and more specifically up to at least 70 °C and the concentrated mixture is heated up to at least 40 °C and more specifically up to at least 50 °C.

The DCDP includes at least one auxiliary concentrate mixture recirculation step CMRS-Aux in which the control unit 24 sets the corresponding concentrate recirculation path X5 and recirculates the CM to homogenize.

Considering the detailed schematic block shown in FIG. 6, the DCDP includes one or more of an auxiliary air evacuation step AES-Aux, an auxiliary priming step of the main fluid line MFPS-Aux an auxiliary recirculation path priming step RPPS-Aux, an auxiliary dissolution liquid fill step DLFS-Aux, an auxiliary concentrate mixture recirculation step CMRS-Aux, and an auxiliary concentration test step CTS-Aux.

The DLFS-Aux may include two different steps, namely an auxiliary first dissolution liquid fill step DLFS′-Aux and an auxiliary second dissolution liquid fill step DLFS″-Aux.

Optionally, in the DCDP, at least the AES-Aux, the DLFS-Aux (including the DLFS′-Aux and the DLFS″-Aux) and the CMRS-Aux, are executed, in time sequence, by the control unit 24.

The dry concentrate dissolution procedure of the dry concentrate in the auxiliary concentrate container 3 will be described in more detail with reference to the figure sequence 1G to 1K.

With reference to the AES-Aux shown in FIG. 1G, the control unit 24 commands the plurality of valves to set an auxiliary air evacuation flow path X1 to evacuate air from the auxiliary concentrate container 3 and commands the pump 8 to remove air from such auxiliary concentrate container 3.

In the auxiliary air evacuation flow path X1, the plurality of valves is configured to allow the air contained in the auxiliary concentrate container 3 to flow into the auxiliary concentrate container outlet line 40 and into a de-aeration chamber (see FIG. 4B) or into the de-aeration chamber 33 (see FIG. 4C) when the pump 8 is configured to operate. The auxiliary concentrate container outlet valve VAO acting on the auxiliary concentrate container outlet line 40 and the drain valve VDR acting on the drain outlet line 30 are open, while the other valves of the plurality of valves configured to operate on the fluid circuit 14 are closed.

Advantageously, during the AES-Aux the control unit 24 operates the pump 8 at a pre-set pumping rate for a pre-fixed time period and stops the pump 8 after the pre-fixed time period has lapsed. During AES-Aux pumping is performed until low level in the deaeration chamber is ensured. In the illustrative embodiment for testing purposes, the pre-set pumping rate was about 300 ml/min and the pre-fixed time period was 20s.

The MFPS-Aux is controlled by the control unit 24 which commands the plurality of valves to set an auxiliary main fluid line prime path and commands the pump 8 to prime the main fluid line 4, particularly after the auxiliary air evacuation step AES-Aux.

Since the auxiliary main fluid line prime path is identical to the main fluid line prime path Z2 shown in FIG. 1B, reference Z2, as well as, FIG. 1B are used for identifying the auxiliary main fluid line prime path EAS-Aux. In particular, in the auxiliary main fluid line prime path the valves of the plurality of valves are configured to allow a dissolution liquid DL, e.g., water, to enter into the main fluid line 4 and flow towards a drain outlet line 30 under the action of the pump 8.

In this case, the water inlet valve VWI placed on the main fluid line 4 and the drain valve VDR acting on the drain outlet line 30 are open, while the other valves of the plurality of valves, different from the water inlet valve VWI and the drain valve VDR, are maintained closed.

During the MFPS-Aux the control unit 24 operates the pump 8 at a pre-set pumping rate up to a pre-fixed pumping volume and stops the pump 8 after the pre-fixed pumping volume has been reached. The pump 8 is operated until the pre-fixed pumping volume greater that the main fluid line volume from water inlet valve VWI to sixth junction point j6 is achieved.

Moving to FIG. 1H, the RPPS-Aux is controlled by the control unit 24 which commands the valves of the plurality of valves to set an auxiliary recirculation priming path X2 and commands the pump 8 to prime the auxiliary recirculation priming path X2, particularly after the AES-Aux and after the MFPS-Aux.

In the auxiliary recirculation priming path X2 the valves of the plurality of valves are configured to allow fluid circulation from the sixth junction point j6 on the main fluid line 4 towards the auxiliary concentrate container inlet line 37, into an auxiliary first concentrate mixing line 41 to a second junction point j2 on the main fluid line 4, in at least a portion of the main fluid line 4 on which the pump 8 is active, to the sixth junction point j6. In particular, the valves of the plurality of valve are configured to allow fluid circulation through the auxiliary concentrate container inlet valve VAI acting on the auxiliary concentrate container inlet line 37 and the second concentrate mixing valve VAM acting on the second concentrate mixing line 41 when such valves VAI, VAM are maintained open, while the other valves of the plurality of valves different from the auxiliary concentrate container inlet valve VAI and the second concentrate mixing valve VAM and configured to operate on the fluid circuit 14, are maintained closed.

During the RPPS-Aux the control unit 24 operates the pump 8 at a pre-set pumping rate up to a pre-fixed pumping volume and stops the pump 8 after the pre-fixed pumping volume has been reached. The pre-fixed pumping volume to pump is lower than the main fluid line volume between fourth junction point j4 and sixth junction point j6, but also higher than the volume in the rest of the recirculation priming path Z3 (i.e., total volume of the recirculation priming path minus the main fluid line volume between fourth junction point j4 and sixth junction point j6).

As shown in FIG. 1I, the DLFS′-Aux is controlled by the control unit 24 which commands the valves of the plurality of valves to set the auxiliary first dissolution liquid fill path X3 and commands the pump 8 to fill a fraction of an expected total dissolution liquid volume in the auxiliary concentrate container 3, particularly after the AES-Aux, after the MFPS-Aux and after the RPPS-Aux. More specifically, in the auxiliary first dissolution liquid fill path X3 the valves of the plurality of valves are configured to allow DL to enter the auxiliary concentrate container 3.

Preferably, the auxiliary first dissolution liquid fill path X3 comprises the main fluid line 4, the auxiliary concentrate container inlet line 37, the first auxiliary container access 45, the first port 9 of the auxiliary concentrate container 3, the auxiliary concentrate container 3, the second port 10 of the auxiliary concentrate container 3, the auxiliary concentrate container outlet line 40, the auxiliary de-aeration chamber 51 not shown in FIG. 1I (but shown in FIG. 4B) and the corresponding vent line, or the de-aeration chamber 33 and the vent 34 of such de-aeration chamber (shown in FIG. 4C). In order to set the auxiliary first dissolution liquid fill path X3, the water inlet valve VWI placed on the main fluid line 4, the auxiliary concentrate container inlet valve VAI on the auxiliary concentrate container inlet line 37, the auxiliary gas valve VGA2 corresponding to the de-aeration chamber 51 (see FIG. 4B) or to the de-aeration chamber 33 (see FIG. 4C—also valve VAO is open in such configuration), are maintained open, while the other valves of the plurality of valves configured to operate on the fluid circuit 14 and different from the valves just mentioned are maintained closed.

During the DLFS′-Aux the control unit 24 operates the pump 8 at a pre-set pumping rate up to a pre-defined pressure and/or a pre-fixed pumping volume. Notably, the DLFS′-Aux includes two steps: in a first water fill step the control unit 24 is configured to stop the pump when said pre-fixed pumping volume has been reached; in a second water fill step the control unit 24 is configured to receive pressure data of a pressure in the first dissolution liquid fill path and to stop the pump when the pressure reaches a pre-defined reference pressure value P2_base. The control unit 24 is configured to end the DLFS′-Aux after a liquid level in the auxiliary de-aeration chamber 51 or in the de-aeration chamber 33 reaches a pre-set liquid high level. For example, in the test embodiment, the pre-set pumping rate was about 300 ml/min and the pre-fixed pumping volume was at least 600 ml, then water was pumped until reaching the pre-defined reference pressure P2_base.

During the DLFS′-Aux, the control unit 24 operates the heater 23 on the main fluid line 4 to heat the DL up to a temperature sufficient to support efficient dissolution in later steps.

Moreover, during the DLFS′-Aux the control unit 24 receives pressure data of a pressure in the auxiliary first dissolution liquid fill path X3 from the pressure sensor P2. Advantageously, the pressure data is stored in a memory associated with the control unit 24. In particular a reference pressure value (P2_base) is stored in such memory.

Moving to FIG. 1J, the DLFS″-Aux is controlled by the control unit 24 which commands the plurality of valves to set an auxiliary second dissolution liquid fill path X4 and commands the pump 8 to fill up to an expected total dissolution liquid volume in the auxiliary concentrate container 3, after the DLFS′-Aux.

In the auxiliary second dissolution liquid fill path X4, the valves of the plurality of valves are configured to allow dissolution liquid DL, i.e., water arriving from the inlet point 5, to enter the auxiliary concentrate container 3. In particular, the auxiliary second dissolution liquid fill path X4 comprises the main fluid line 4, the sixth junction point j6, the auxiliary concentrate container inlet line 37, the first auxiliary container access 45, the first port 9 of the auxiliary concentrate container 3, the auxiliary concentrate container 3, the second port 10 of the auxiliary concentrate container 3, the second auxiliary container access 46, the auxiliary concentrate container outlet line 40, the auxiliary de-aeration chamber 51 not shown in FIG. 1J (see FIG. 4B) or the de-aeration chamber 33 (see FIG. 4C), and a part of the auxiliary concentrate container outlet line 40 upstream the auxiliary concentrate container outlet valve VAO. To set the auxiliary second dissolution liquid fill path X4, the water inlet valve VWI placed on the main fluid line 4 and the auxiliary concentrate container inlet valve VAI placed on the auxiliary concentrate container inlet line 37 are open, while the other valves of the plurality of valves different from the water inlet valve VWI and the auxiliary concentrate container inlet valve VAI are closed.

In particular, during the DLFS″-Aux the control unit 24 operates the pump 8 at a pre-set pumping rate up to a pre-fixed pressure in the auxiliary second dissolution liquid fill path X4 and stops the pump 8 after the pre-fixed pressure has been reached; optionally the pre-fixed pressure is a reference pressure value (P2_base) in the auxiliary second dissolution liquid fill path X4 plus an overpressure value of e.g., 40 mmHg.

Furthermore, during the DLFS″-Aux the control unit 24 operates a heater 23 on the main fluid line 4 to heat the dissolution liquid up to e.g., at least 70 °C and optionally up to at least 80 °C.

As shown in FIG. 1K, the CMRS-Aux is controlled by the control unit 24 which commands the plurality of valves to set an auxiliary concentrate recirculation path X5 and commands the pump 8 to re-circulate the CM to dissolve the DC and homogenously mix the concentrated mixture CM. The CMRS-Aux is performed after the DLFS′-Aux and after the DLFS″-Aux. During the CMRS-Aux the control unit 24 operates the pump 8 at a pre-set pumping rate for a pre-fixed time period and stops the pump 8 after the pre-fixed time period has lapsed.

Furthermore, during the CMRS-Aux the control unit 24 operates a heater 23 on the main fluid line 4 to heat the CM up to a temperature sufficient to support efficient dissolution in later steps.

Advantageously, the control unit 24 is configured to perform the auxiliary concentration test step CTS-Aux to measure a concentration of the CM and to compare the measured concentration with an expected concentration of the CM. Optionally, the control unit 24 is configured to measure the concentration during the CMRS-Aux.

### Dry Concentrate Dissolution Procedure Performed on Both Concentrate Containers in Series

Should the DCDP for dissolving and/or diluting a dry concentrate be performed in each of the concentrate containers 2, 3 of the fluid medical generation apparatus 1 according to the embodiment shown in FIG. 1, the DCDP would firstly be run, by the control unit 24, on a single concentrate container 2, 3, and, secondly, on the other concentrate container 2, 3, interposing one or more emptying and/or rinsing and/or priming steps on at least of the main fluid line 4 of the fluid circuit 14.

In particular, considering a starting situation wherein a dry concentrate is housed both in the primary concentrate container 2 and in the auxiliary concentrate container 3, the DCDP is run either according to the DCDP aforementioned in relation to the primary concentrate container 2 or according to the DCDP aforementioned in relation to the auxiliary concentrate container 3 to form an auxiliary liquid concentrate. Then, at least one interposed step, like emptying and/or rinsing and/or priming at least the main fluid line 4 of the fluid circuit 14, occurs.

Generally, to achieve it, the water inlet valve VWI and the drain valve VDR are moved, by the control unit 24, to the open condition, while all the other valves of the plurality of valves of the fluid circuit 14 are maintained closed, and the pump 8 is activated to provide the water flow from the inlet point 5 to the drain port 29. Once the interposed step is ended, the DCDP may be run again on the concentrate container 2, 3 not yet involved in the DCDP to form the primary liquid concentrate. A further interposed step, like the previous one, may be performed to prepare to a subsequent liquid concentrate mixing procedure LCMP.

The DCDP is necessary every time a DC is housed inside a concentrate container 2, 3, even in a starting situation where one concentrate container 2, 3 houses a DC like in the embodiment shown in FIG. 3, and the other one houses an auxiliary liquid concentrate LC′ which does not require any dissolution operation. According to this starting situation the DCDP is run only in relation to the concentrate container 2, 3 containing the DC because the liquid concentrate housed in the other concentrate container 2, 3 is ready to be diluted and/or mixed during the successive liquid concentrate mixing procedure LCMP.

### Liquid Concentrate Mixing Procedure

Mixing to obtain the final PD fluid is always performed from liquid concentrates LC, either pre-manufactured or prepared from dry concentrates DC per above.

In summary, the mixing sequence is a three-step method where first the correct mixture of liquid concentrate (main electrolytes and buffer) from auxiliary concentrate container 3 added with water is established during two steps with conductivity feedback and then the other liquid concentrate (glucose) from the primary concentrate container 2 is added volumetrically in the final step.

The actual concentration of the concentrates needs to be known to enable volumetric dosing in the mixing sequence. To test the concentration of the glucose concentrate and the electrolyte concentrate, the following sequence may be used:
1. Fill a volume of water to the mixing container;
2. Fill a known fraction of the water (W) volume of electrolyte concentrate (E) to the mixing container;
3. Mix the solution by recirculation and, as homogeneity is detected (e.g., conductivity variance below threshold), sense the conductivity;
4. Calculate the actual concentration of the electrolyte concentrate based on the sensed conductivity and the E to W volume ratio;
5. Add a known fraction of the W+E volume of glucose concentrate to the mixing container;
6. Mix the solution by recirculation and, as homogeneity is detected (e.g., conductivity variance below threshold), sense the conductivity;
7. Calculate the actual concentration of the glucose concentrate based on the sensed conductivity and the conductivity and volume before the glucose addition.

An initial recipe with nominal volumes of water and the concentrates is calculated based on the desired batch volume and glucose concentration set by the user. Also, nominal conductivities for the different mixing steps are calculated at this point. The nominal volume of A concentrate is then added to the mixing container along with 80% of the nominal water volume. After recirculation mixing to homogeneity, the residual water and glucose concentrate volumes to add are recalculated based on the conductivity's deviation from nominal. This method makes the mixing system robust towards deviations in the A concentrate concentration.

A principal difference between the flexible mixing container/bag 11 (see FIGS. 2-4) and a rigid mixing container 11 (see FIG. 1) is that the flexible mixing container/bag can be emptied from fluid without letting air in. The rigid mixing container however requires that the emptied fluid is replaced with air. This means that after complete drain of the flexible mixing bag 11, the recirculation fluid path will contain a certain water volume. This dead volume must be accounted for by subtracting it from the first water volume to add during mixing. Note that when a new mixing bag that contains air is attached, it needs to be emptied from air and the air that becomes present downstream the mixing bag must be replaced with water. In the rigid tank system, air is replacing the drained fluid and it is hence possible to completely drain the recirculation fluid path. The dead volume will in this case be close to zero and may not need to be accounted for.

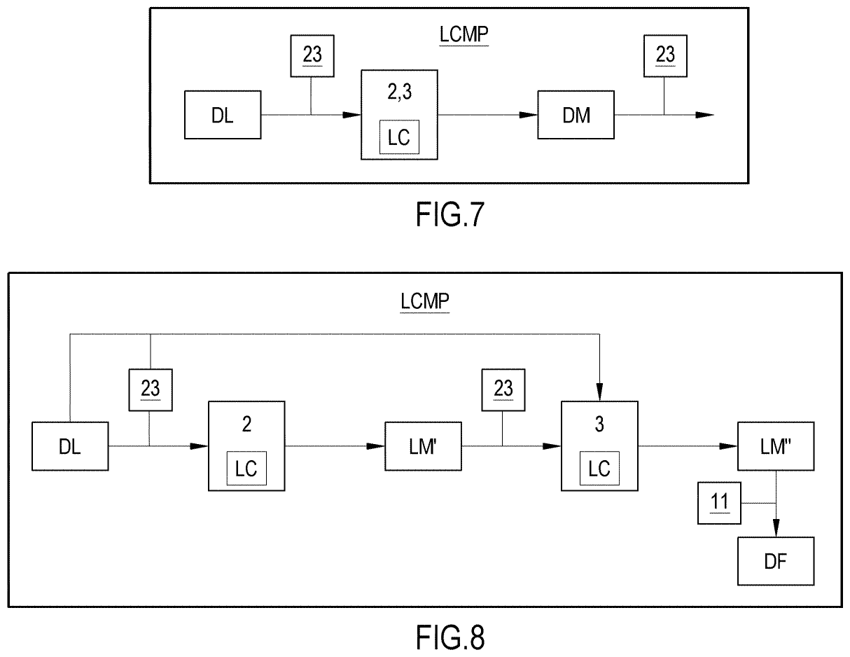

Moving back to the figures and the procedure, as schematically shown in FIG. 7, the control unit 24 is also configured to run a liquid concentrate mixing procedure LCMP which includes sending an auxiliary liquid concentrate LC′ from either the primary concentrate container 2 or the auxiliary concentrate container 3 to the mixing container 11. The liquid concentrate mixing procedure LCMP further comprises sending a dissolution liquid DL, e.g., water, to the mixing container 11 to dilute the liquid concentrate to a desired dilution value and then to send a primary liquid concentrate LC″ from the other one of the primary concentrate container 2 and the auxiliary concentrate container 3. After properly mixing the liquids a dialysis fluid is obtained.

In general, the liquid concentrate mixing procedure LCMP includes heating the dilution liquid DL (namely water) and/or the liquid mixture DM by the heater 23. Advantageously, the DL is heated up to at least 30 °C and more specifically up to at least 37 °C and the diluted liquid mixture DM are heated up to at least 30 °C and more specifically up to at least 37 °C.

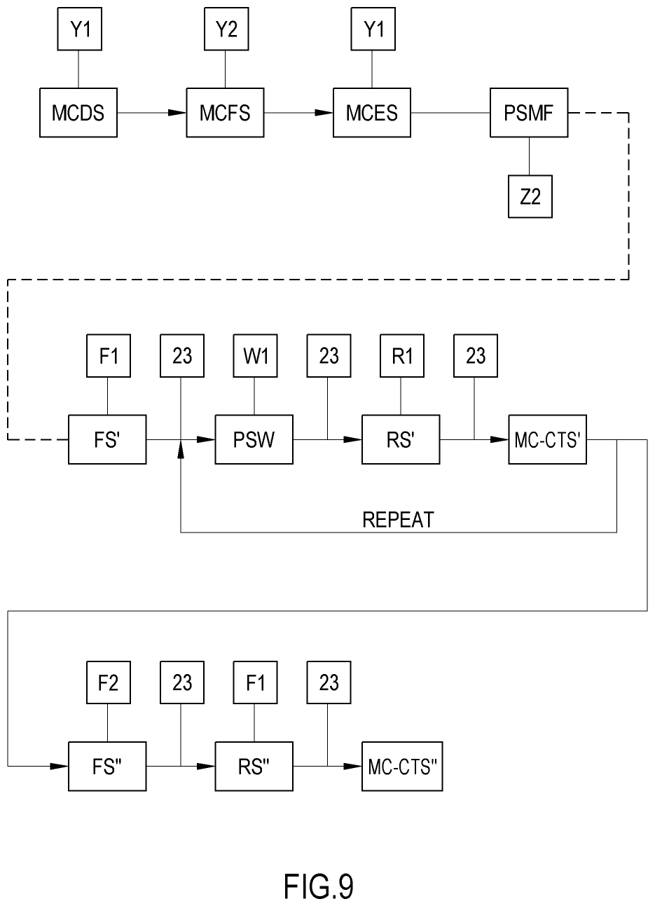

More specifically, as schematically shown in FIG. 9, the liquid concentrate mixing procedure LCMP includes at least one diluted mixture recirculation step RS in which the control unit 24 sets a mixing recirculation path R1 and recirculates the corresponding diluted liquid mixture DM to homogenize.

In particular, the liquid concentrate mixing procedure LCMP includes one or more of the following steps:
a mixing container drain step MCDS;
a mixing container filling step for rinse MCFS;
a priming step of the main fluid line PSMF;
a first filling step FL′ of an auxiliary liquid concentrate LC′ (or of a first diluted mixture DM′) arriving from the auxiliary concentrate container 3 into the mixing container 11, the first filling step further including sending water to the mixing container to dilute the auxiliary liquid concentrate LC′;
a first recirculation step RS' of the first diluted liquid mixture DM′ (auxiliary liquid concentrate LC′ added with water);
a second filling step FS″ of a primary liquid concentrate LC″ arriving from the primary concentrate container 2, into the mixing container 11 to form a dialysis mixture DiaM;
a second recirculation step RS″ of the dialysis mixture DiaM to form the (homogeneous) dialysis fluid DF.

Reference to the sequence of figures from 1L to 1Q is now made.

As shown in FIG. 1L, the MCDS is controlled by the control unit 24 which commands the valves of the plurality of valves to set a mixing container drain path Y1 to allow the drain of the mixing container 11 and commands the pump 8 to move any fluid inside the mixing container 11 through the outlet 13, at least one portion of the main fluid line 4 on which the pump 8 is configured to operate, the drain outlet line 30 and the drain port 29. The mixing container drain path Y1 (FIG. 1L) comprises the mixing container 11, the outlet 13, the mixing container outlet line 16 connecting the outlet 13 of the mixing container 11 to a first junction point j1 on the main fluid line 4, the first junction j1, the at least one portion of the main fluid line 4 on which the pump 8 is active from the first junction point j1 to a sixth junction point j6, the drain outlet line 30 from the sixth junction point j6 to a drain port 29 and such a drain port 29. Furthermore, the mixing container drain path Y1 comprises the mixing container outlet valve VMO configured to operate on the mixing container outlet line 16 and the drain valve VDR configured to operate on the drain outlet line 30. When the control unit (24) controls the plurality of valves to set the mixing container drain path Y1, the mixing container outlet valve VMO and the drain valve VDR are open to let the fluid flow from the mixing container 11 to the drain port 29, while the other valves of the plurality of valves configured to operate on the fluid circuit 14 different from the mixing container outlet valve VMO and the drain valve VDR are closed. Preferably, the control unit 24 is configured to first set the mixing container drain path Y1 prior setting the mentioned concentrate recirculation path Z6, X5.

In particular, the mixing container drain path Y1 comprising the MCDS, is set prior every other path, when the medical fluid generation apparatus 1 needs to start a new cycle for producing a dialysis fluid DF. This occurs because at the beginning of each producing cycle, it is necessary to completely drain the mixing container 11 to perform at least one successive cleaning procedure which aims to have the mixing container 11 configured for receiving the auxiliary liquid concentrate LC′, water and/or the diluted liquid mixtures DM′, to be mixed for producing the desired dialysis fluid DF.

During the MCDS the control unit 24 operates to achieve emptying of the mixing container 11. In case of a flexible mixing container 11, the control unit 24 operates the pump 8 at a pre-set pumping rate and stops the pump 8 when a pre-fixed pressure in the mixing container drain path Y1 has been reached. In the illustrative embodiment for testing purposes, the pre-set pumping rate was set to about 300 ml/min and the pre-fixed pressure was equal to or lower than-100 mmHg. Differently, in case a rigid mixing container 11 with open air vent is used, the conductivity meter 25 may be used to detect when air enters it. In this situation, the conductivity falls drastically to near zero. Alternatively, MCDS starts with the air vent closed and using the gas law the head space may be calculated. Knowing the total volume of mixing container 11 allows to calculate how much volume to remove for emptying.

Moving to FIG. 1M, the MCFS is part of a cleaning procedure of the mixing container 11. In particular, the MCFS comprises the control unit 24 driving the valves of the plurality of valves to set a mixing container filling path Y2 to rinse at least the mixing container inlet line 15 and the mixing container 11. The MCFS also comprises the control unit 24 driving the pump 8 to move the fluid arriving from at least one portion of the main fluid line 4 on which the pump 8 is active to the mixing container inlet line 15 and the mixing container 11, in particular from the inlet point 5 of the main fluid line 4 of the fluid circuit 14 to the mixing container inlet line 15 and the mixing container 11.

As highlighted in FIG. 1M, the mixing container filling path Y2 comprises at least one portion of the main fluid line 4 of the fluid circuit 14, particularly the whole main fluid line 4 of the fluid circuit 14, on which the pump 8 is active, the sixth junction point j6, the mixing container inlet line 15 and the mixing container 11. Moreover, the mixing container filling path Y2 comprises the water inlet valve VWI configured to operate on the main fluid line 4 and the mixing container inlet valve VMI configured to operate on the mixing inlet line 15. When the control unit 24 controls the plurality of valves to set the mixing container filling path Y2, at least the water inlet valve VWI and the mixing container inlet valve VMI are maintained open to let the fluid flow from the fluid main line 4 to the mixing container 11. The other valves of the plurality of valves configured to operate on the fluid circuit 14 that may let fluid enter or exit from the mixing container filling path Y2, are closed. In case a rigid mixing container 11 is used, also air valve 22 should be left open. Preferably all the other valves of the plurality of valves configured to operate on the fluid circuit 14 different from the water inlet valve VWI, the mixing container inlet valve VMI, and the air valve 22 are closed. In case a flexible mixing container 11 no air vent is necessary and therefore all the other valves of the plurality of valves configured to operate on the fluid circuit 14 different from the water inlet valve VWI and the mixing container inlet valve VMI are closed.

During the mixing container filling step for rinse MCFS, the control unit 24 operates the pump 8 at a pre-set pumping rate up to a pre-fixed pumping volume and stops the pump 8 after the pre-fixed pumping volume has been reached. The pre-fixed pumping volume is sufficient to achieve proper rinse of the main fluid line (4) and the mixing container (11). In the illustrative embodiment for testing purposes, the pre-set pumping rate was about 330 ml/min and the pre-fixed pumping volume was at least 100 ml.

As shown in FIG. 1L, the MCFS comprises a mixing container emptying step MCES wherein the control unit 24 commands the valves of the plurality of valves to set a mixing container emptying path (which corresponds to the mixing container drain path Y1 highlighted in FIG. 1L) to rinse at least the mixing container 11 and mixing container outlet line 16. In particular, the mixing container emptying step MCES also comprises the control unit 24 configuring the pump 8 active on the main fluid line 4 to move the fluid arriving from the mixing container 11 to the drain outlet line 30 and the drain port 29. The mixing container emptying path is localized downstream the mixing container filling path Y2 and comprises the mixing container outlet line 16, the first junction point j1 on the main fluid line 4, at least one portion of the main fluid line 4, the sixth junction point j6, the drain outlet line 30 and the drain port 29.

The mixing container emptying path further comprises the mixing container outlet valve VMO configured to operate on the mixing container outlet line 16 and the drain valve VDR configured to operate on the drain outlet line 30. When the control unit 24 controls the plurality of valves to set the mixing container emptying path, at least the mixing container outlet valve VMO and the drain valve VDR are maintained open to let the fluid flow from the mixing container 11 to the drain port 29. The other valves of the plurality of valves configured to operate on the fluid circuit 14 that may let fluid enter into or exit from the mixing container emptying path are maintained closed.

The mixing container emptying step MCES is controlled, preferably by the control unit 24, after the MCFS. The mixing container emptying step MCES allow the cleaning procedure to close its own cycle. Both the MCFS and the mixing container emptying step MCES are preferably performed at the beginning of the dialysis fluid preparation cycle to clean the mixing inlet line 15, the mixing container 11, the mixing outlet line 16 and the main fluid line 4.

During the mixing container draining step, the control unit 24 operates to achieve emptying of the mixing container 11. In case of a flexible mixing container 11, the control unit 24 operates the pump 8 at a pre-set pumping rate and stops the pump 8 when a pre-fixed pressure in the mixing container emptying path has been reached. In the illustrative embodiment for testing purposes, the pre-set pumping rate was about 330 ml/min and the pre-fixed pressure, particularly detected on the mixing outlet line 16, was equal to or lower than-100 mmHg. Differently, in case a rigid mixing container 11 with open air vent is used, the conductivity meter 25 may be used to detect when air enters it. In this situation, the conductivity falls drastically to near zero. Alternatively, MCDS starts with the air vent closed and using the gas law the head space may be calculated. Knowing the total volume of mixing container 11 allows to calculate how much volume to remove for emptying.

The priming step of the main fluid line PSMF comprises the control unit 24 driving the valves of the plurality of valves to set a main fluid priming path (which corresponds to the main fluid path Z2 highlighted in FIG. 1B) to rinse the main fluid line 4. The PSMF also comprises the control unit 24 driving the pump 8 to move the fluid arriving from the inlet point 5 at least to the drain outlet line 30. In particular, the main fluid priming path comprises at least one portion of the main fluid line 4, particularly the whole main fluid line 4 of the fluid circuit 14, on which the pump 8 is configured to operate. Preferably, the main fluid priming path develops between the inlet point 5 and the outlet drain line 30, specifically between the inlet point 5 and the drain port 29.

More in detail, the main fluid priming path comprises the water inlet valve VWI configured to operate on the main fluid line 4 and the drain valve VDR configured to operate on the outlet drain line 30. When the control unit 24 controls the plurality of valves to set the main fluid priming path, at least the water inlet valve VWI and the drain valve VDR are maintained open to let the fluid flow from the fluid main line 4 to the drain outlet line 30. The other valves of the plurality of valves configured to operate on the fluid circuit 14 are maintained closed.

During the PSMF, the control unit 24 operates the pump 8 at a pre-set pumping rate and until a pre-fixed time period has lapsed. The pump 8 is run at least until after a volume contained between first junction point j1 and sixth junction point j6 has been pumped with the aim that any residual conductivity is low enough to not significantly impact the composition accuracy in the following steps. In the experimental embodiment for the test, the pre-set pumping rate was about 330 ml/min and the pre-fixed time period was 30s. Furthermore, during the PSMF, the control unit 24 optionally operates the heater 23 on the main fluid line 4 to heat the fluid flowing through the main fluid priming path up to a value allowing to achieve a precise conductivity measurement, for example 37 °C.

During the PSMF, the control unit 24 is configured to perform a main fluid line concentration test step MF-CTS to measure a concentration value of the fluid flowing through the main fluid priming path. Optionally, the control unit 24 is configured to compare the concentration value measured in the fluid flowing through the main fluid priming path and a concentration reference value to ensure that only water is present in the fluid path; particularly the measured concentration value should not exceed 0.20 mS/cm, preferably not exceed 0.15 mS/cm.

The PSMF may be performed just after the mixing container emptying step MCES and/or the MCFS or may correspond to the MFPS performed in relation to the mentioned DCDP and/or the any dilution procedure of a liquid concentrate CM.

Moving to FIG. 1N, the mentioned FL′ of the first diluted liquid mixture DM′, comprises the control unit 24 controlling the valves of the plurality of valves to set an auxiliary concentrate filling path F1 to fill the mixing container 11 with the auxiliary liquid concentrate LC′, particularly according to a pre-fixed nominal volume. The FS' of first diluted mixture DM′ also comprises the control unit 24 driving the pump 8 to move such liquid concentrate from the corresponding concentrate container 2, 3, particularly from the auxiliary concentrate container 3, to the mixing container 11.

More specifically, the auxiliary concentrate filling path F1 (FIG. 1N) comprises the second concentrate container inlet line 37, the second concentrate mixing line 41, the second junction point j2, at least one portion of the main fluid line 4 in which the pump 8 is active, the sixth junction point j6, the mixing container mixing line 15, the inlet 12 of the mixing container 11 and the mixing container 11. Furthermore, the auxiliary concentrate filling path F1 comprises the second concentrate mixing valve VAM configured to operate on the second concentrate mixing line 41 and the mixing container inlet valve VMI configured to operate on the mixing inlet line 15. When the control unit 24 controls the plurality of valves to set the auxiliary concentrate filling path F1, the second concentrate mixing valve VAM and the mixing container inlet valve VMI are maintained open to let the first diluted liquid mixture DM′ arriving from the auxiliary concentrate container 3 to move to the mixing container 11. All the other valves of the plurality of valves configured to operate on the fluid circuit 14 that may let fluid enter into or exit from the auxiliary concentrate filling path F1 are maintained close.

Alternatively, second auxiliary container access 46 and auxiliary concentrate container outlet line 40 up to third junction point j3 may be used as part of the auxiliary concentrate filling path F1 instead of second concentrate container inlet line 37, the second concentrate mixing line 41 and second junction point j2. In this case, first port 9 of auxiliary concentrate container 3 is to be directly connected to second auxiliary container access 46 to avoid air to enter into the lines.

During the FS' of the first diluted mixture DM′, the control unit 24 operates the pump 8 at a pre-set pumping rate up to a pre-fixed pumping volume and stops the pump 8 after the pre-fixed pumping volume has been reached. Optionally, the pre-set pumping rate being about 220 ml/min and the pre-fixed pumping volume is equal to or higher than the pre-fixed nominal volume (from the recipe) of the auxiliary liquid concentrate LC′. Further during the FS' of the first diluted mixture DM′, the control unit 24 operates the heater 23 on the main fluid line 4 to heat the auxiliary liquid concentrate LC′ flowing through the auxiliary concentrate filling path F1 up to 37 °C. The first filling step FS' of the first diluted mixture DM′ is controlled, preferably by the control unit 24, after the MCDS, after the MCFS, after the mixing container emptying step MCES and after the PSMF.

As shown in FIG. 1O, the first filling step FS' of the first diluted mixture DM′ is followed by at least one filling step of water FSW comprising the control unit 24 driving the valves of the plurality of valves to set a water filling path W1 to fill the mixing container 11, at least partially, with a pre-fixed volume of water. The filling step of water FSW also comprises the control unit 24 driving the pump 8 to flow the water arriving from the inlet point 5 of the fluid circuit 14 to the mixing container 11. In particular, the water filling path W1 comprises at least one portion of the main fluid line 4, particularly developing between the inlet point 5 of the fluid circuit 14 and the sixth junction point j6, the sixth junction point j6, the mixing container inlet line 15, the inlet 12 of the mixing container 11 and the mixing container 11. More particularly, the water filling path W1 comprises the water inlet valve VWI configured to operate on the fluid main line 4 and the mixing container inlet valve VMI configured to operate on the mixing inlet line 15. When the control unit 24 controls the plurality of valves to set the water filling path W1, the water inlet valve VWI and the mixing container inlet valve VMI are maintained open to let the water arriving from the inlet point 5 of the fluid circuit 14 to flow to the mixing container 11. The other valves of the plurality of valves configured to operate on the fluid circuit 14 that may let fluid enter into or exit from the water filling path W1 are maintained close. Preferably all the other valves of the plurality of valves configured to operate on the fluid circuit 14 different from the water inlet valve VWI and the mixing container inlet valve VMI are maintained closed.

During the filling step of water FSW, the control unit 24 operates the pump 8 at a pre-set pumping rate up to a pre-fixed pumping volume (from the recipe) and stops the pump 8 after said pre-fixed pumping volume has been reached. Optionally, the pre-fixed pumping volume is maintained at a predetermined fraction of the total nominal volume minus the dead volume, such as about 80% of a calculated reference volume of water. The calculated reference volume of water is obtained by a pre-fixed nominal volume of water minus a dead volume of water. The dead volume of water is defined by a volume including the main fluid line volume from the first junction point j1 to the sixth junction point j6, the volume of tubing between the sixth junction point j6 and the intermediate point 37a of the auxiliary concentrate container inlet line 37, the volume of tubing between the intermediate point 37a of the auxiliary concentrate container inlet line 37 and the second junction point j2, and the volume of tubing between the sixth junction point j6 and the inlet 12 of mixing container 11. Additionally, the dead volume of water includes the volume of the mixing container outlet line 16 (from outlet 13 to first junction point j1) in case the mixing container 11 is a flexible bag (vice versa, the mixing container outlet line 16 is empty in case the mixing container is a rigid container 11 and is not to be taken into account).

During the filling step of water FSW, the control unit 24 operates the heater 23 on the main fluid line 4 to heat the water flowing through the filling path of water W1 up to 37 °C.

Moving now to FIG. 1P, the RS' of the first diluted mixture DM′ comprises the control unit 24 driving the valves of the plurality of valves to set the mixing recirculation path R1 to mix the first diluted mixture DM′ (auxiliary liquid concentrate LC′ plus added water) filled into the mixing container 11.

The RS' of the first diluted mixture DM′ also comprises the control unit 24 driving the pump 8 to flow such liquids through the mixing recirculation path R1. The mixing recirculation path R1 comprises the mixing container 11, the outlet 13 of the mixing container 11, the mixing container outlet line 16 and the first junction point j1, at least one portion of the main fluid line 4 on which the pump 8 is configured to operate, particularly developing between the first junction point j1 and the sixth junction point j6, the sixth junction point j6 and the mixing container inlet line 15.

The mixing recirculation path R1 comprises the mixing container outlet valve VMO configured to operate on the mixing outlet line 16 and the mixing container inlet valve VMI configured to operate on the mixing inlet line 15. When the control unit 24 controls the plurality of valves to set the mixing recirculation path R1, the mixing container outlet valve VMO and the mixing container inlet valve VMI are maintained open to let the first diluted mixture DM′ to flow through the mixing recirculation path R1. The other valves of the plurality of valves configured to operate on the fluid circuit 14 that may let fluid enter into or exit from the mixing recirculation path R1 are maintained closed.

During the first recirculation step R1 of the first diluted mixture, the control unit 24 operates the pump 8 at a pre-set pumping rate and at least for a pre-fixed time period has lapsed; in the illustrative embodiment for testing purposes, said pre-set pumping rate was about 330 ml/min and said pre-fixed time period was 60s. Moreover, during the first recirculation step R1 of the first diluted mixture DM′, the control unit 24 may operate the heater 23 on the main fluid line 4 to heat the first diluted mixture DM′ up to 37 °C.

In order to check the conductivity of the first diluted mixture DM′, the control unit 24 is configured to perform a further main fluid line concentration test step ML-CTS' by which it is possible to detect, preferably continuously, the conductivity changes of the first diluted mixture DM′ flowing through the mixing recirculation path R1. Optionally, the control unit 24 is configured to compare the median conductivity detected in the first diluted mixture DM′, by means of at least one conductivity meter 25, 26, flowing through the mixing recirculation path R1 with a pre-fixed threshold. When the variation the conductivity (namely conductivity variance) is below a predefined threshold, a signal of interruption of the RS' is emitted, and managed by the control unit 24 to stop recirculation since the proper homogenization has been reached. Furthermore, measured median conductivity is used to recalculate the residual water volume to add to the first diluted mixture DM′ and the volume of the other concentrate to add.

Advantageously, the filling step of water FSW and the RS' of the first diluted mixture DM′ is repeated at least once. The purpose is to add a recalculated residual water volume in order to achieve nominal conductivity of the first diluted mixture DM′.

During the repetition of the filling step of water, the control unit 24 operates the pump 8 at a pre-set pumping rate up to a pre-fixed pumping volume and stops the pump 8 after the pre-fixed pumping volume, equal to a recalculated reference volume of water, has been reached. In the illustrative embodiment for testing purposes, the pre-set pumping rate was about 330 ml/min. During the repetition of the filling step of water FSW, the control unit 24 operates the heater 23 on the main fluid line 4 to heat the water and/or the first diluted mixture flowing through the water filling path and/or the mixing recirculation path up to a value allowing to achieve a precise conductivity measurement e.g., 37 °C. During the repetition of the RS' of the first diluted mixture DM′, the control unit 24 operates the pump 8 at a pre-set pumping rate and at least until a pre-fixed time period has lapsed. Again, in the illustrative embodiment for testing purposes, the pre-set pumping rate was about 330 ml/min and the pre-fixed time period was 60s. Further, during the repetition of the RS' of the first diluted mixture DM′, the control unit 24 operates the heater 23 on the main fluid line 4 to heat the first diluted mixture DM′ up to a value allowing to achieve a precise conductivity measurement e.g., 37 °C.

Advantageously, the control unit 24 is configured to perform a further main fluid line concentration test step MF-CTS' to detect, by means of at least one conductivity meter 25, 26, preferably continuously, the conductivity of the first diluted mixture DM′ flowing through the repetition of the mixing recirculation path R1.

The control unit 24 is configured to compare the conductivity variance detected in the first diluted mixture DM′ flowing through the mixing recirculation path R1 with a pre-fixed threshold, and to send a signal of interruption of the RS' when the variance is below the set threshold.

The second filling step FS″ of the primary liquid concentrate LC″ is run to form a dialysis mixture DiaM as follows. The second filling step FS″ comprises the control unit 24 driving the valves of the plurality of valves to set a primary concentrate filling path F2 (FIG. 1Q) to fill the mixing container 11 with primary liquid concentrate LC″ arriving from the primary concentrate container 2.

The second filling step FS″ of the primary liquid concentrate LC″ comprises the control unit 24 driving the pump 8 to move such liquids from the primary concentrate container 2 to the mixing container 11. In particular, the primary concentrate filling path F2 comprises an initial portion of the primary concentrate container inlet line 31, the first concentrate mixing line 36, the fourth junction point j4, at least one portion of the main fluid line 4 in which the pump 8 is active, the sixth junction point j6, the mixing container inlet line 15, the inlet 12 of the mixing container 11 and the mixing container 11. The primary concentrate filling path F2 comprises the first concentrate mixing valve VGM configured to operate on the first concentrate mixing line 36 and the mixing container inlet valve VMI configured to operate on the mixing inlet line 15. When the control unit 24 controls the plurality of valves to set the primary concentrate filling path F2, the first concentrate mixing valve VGM and the mixing container inlet valve VMI are maintained open to let primary liquid concentrate LC″ arriving from the primary concentrate container 2 to move towards the mixing container 11. The other valves of the plurality of valves configured to operate on the fluid circuit 14 are maintained closed. Preferably, all the other valves of the plurality of valves configured to operate on the fluid circuit 14 different from the first concentrate mixing valve VGM and the mixing container inlet valve VMI are maintained closed.

During the second filling step FS″ of primary liquid concentrate LC″, the control unit 24 operates the pump 8 at a pre-set pumping rate up to a pre-fixed pumping volume and stops the pump 8 after the pre-fixed pumping volume has been reached. In the illustrative embodiment for testing purposes, the pre-set pumping rate was about 200 ml/min and the pre-fixed pumping volume was equal to a recalculated reference volume of primary liquid concentrate LC″. During the second filling step FS″ of the primary liquid concentrate LC″ into the mixing container 11, the control unit 24 operates the heater 23 on the main fluid line 4 to heat such liquids DM″, ML″ flowing through the primary concentrate filling path F2 up to a value allowing to achieve a precise conductivity measurement e.g., 37 °C. The second filling step FS″ of the primary liquid concentrate LC″ is executed, preferably by the control unit 24, after the RS′, after the repetition of the filling step of water FSW and after the repetition of the RS' of the first diluted mixture DM′.

The second filling step FS″ of the primary liquid concentrate LC″ is followed by at least one second recirculation filling step RS″ of the dialysis mixture DiaM formed by adding the primary liquid concentrate LC″ to the first diluted mixture DM′. In particular, the RS″ of the dialysis mixture DiaM comprises the control unit 24 driving the valves of the plurality of valves to set a mixing recirculation path R1 and mix the dialysis mixture DiaM into the mixing container 11. The recirculation filling step of the dialysis mixture DiaM also comprises the control unit 24 driving the pump 8 to flow such dialysis mixture DiaM through the mixing recirculation path.

The mixing recirculation path is identical to the first one identified with the reference number R1 and highlighted in FIG. 1P. As can be seen in FIG. 1P, also the mixing recirculation path comprises the mixing container 11, the outlet 13 of the mixing container 11, the mixing container outlet line 16 and the first junction point j1 at least one portion of the main fluid line 4 in which the pump 8 is configured to operate, particularly developing between the first junction point j1 and the sixth junction point j6, the sixth junction point j6, the mixing container inlet line 15 and the inlet 12.

The mixing recirculation path R1 comprises the mixing container outlet valve VMO configured to operate on the mixing outlet line 16 and the mixing container inlet valve VMI configured to operate on the mixing inlet line 15. When the control unit 24 controls the plurality of valves to set the mixing recirculation path, the mixing container outlet valve VMO and the mixing container inlet valve VMI are maintained open to let the dialysis mixture DiaM to flow through the mixing recirculation path. All the other valves of the plurality of valves configured to operate on the fluid circuit 14 that may let fluid enter into or exit from the mixing recirculation path are maintained closed.

During the RS″ of the dialysis mixture DiaM, the control unit 24 operates the pump 8 at a pre-set pumping rate and at least for a pre-fixed time period. In the illustrative embodiment for testing purposes, the pre-set pumping rate was about 330 ml/min and the pre-fixed time period was 60s.

During the second recirculation step of the dialysis mixture DiaM, the control unit 24 may operate the heater 23 on the main fluid line 4 to heat DiaM up to 37 °C.

The control unit 24 is configured to perform an additional main fluid line concentration test step MF-CTS″ to detect, by means of at least one conductivity meter 25, 26, preferably continuously, conductivity of the dialysis mixture DiaM flowing through the mixing recirculation path. The control unit 24 is configured to compare the conductivity variance detected in the dialysis mixture DiaM with a pre-fixed threshold, to issue a signal of interruption of the second recirculation step of the dialysis fluid when variance is below said threshold, since the dialysis mixture DiaM has been transformed into a dialysis fluid FD ready to be used in a dialysis medical treatment.

### Operation

The operation of the medical fluid generation apparatus, mainly described up to this point only in a structural sense, is as follows.

As can been seen in the embodiments shown in FIGS. 1 to 4C, the medical fluid generation apparatus 1 may work both with dry concentrates substances, like powders and/or granulates, and liquid concentrate substances. It is also possible that the medical fluid generation apparatus 1 works with a combination of different types of concentrates, like dry concentrates to be dissolved and diluted and liquid concentrates to be diluted. The medical fluid generation apparatus 1 may also work with different liquid concentrates to be diluted and mixed for forming the desired dialysis fluid.

When dry concentrates are used for preparing the desired dialysis fluid FD, it is necessary to provide a dissolution of such dry concentrates to form corresponding liquid concentrates LC′, LC″ which need to be diluted by adding water, and properly mixed to form the dialysis fluid FD to be used during a dialysis medical treatment.

If one concentrate is dry and other one is liquid, it is necessary to dissolve the dry concentrate adding water and recirculating it properly, as well as to dilute the liquid concentrate in order to mixed together all substances. When both concentrates are liquid, it is only requested to dilute and mix them.

Among the various usable concentrates, it has to be noted that the concentrates to be used should contain appropriate electrolytes, buffer, glucose, and possibly other osmotic agents, e.g., Icodextrin.

In order to use the same fluid paths of the fluid circuit 14 and the same medical fluid generation apparatus' components, the dissolution sequences of the concentrates to be used are preferably performed in series.

When the concentrate to be used comprises an electrolyte and a buffer powder, it is preferred to completely dissolve/dilute such a concentrate to keep control of the concentrate composition. With reference to the glucose, complete dissolution is needed to ensure that the concentration is close enough to the nominal and that the concentration does not change due to continued powder dissolution during therapy. Knowledge of the glucose concentrate concentration is needed since the glucose concentrate is proportioned volumetrically.

Considering for example the embodiment shown in FIG. 1, i.e., the medical fluid generation apparatus 1 provided with the mixing container 11 placed inside the supporting structure 42, it is possible to connect different concentrate containers 2, 3 to the container accesses 43, 44, 45, 46 provided on the supporting structure 42.

Once the medical fluid generation apparatus 1 is provided with the concentrate containers 2, 3, it is necessary to drain the mixing container 11 and the fluid circuit 14 of any residual liquid and/or substance therein and to prime them to allow of performing all the operations to form a dialysis fluid FD. In order to arrange the medical fluid generation apparatus 1 so that it may start to work, some steps of the liquid concentrate mixing procedure LCDP are executed before every other step or operation of the medical fluid apparatus 1. In particular, before starting any dissolution, recirculation and mixing operation, the MCDS is run for draining the mixing container 11, the mixing container outlet line 16, and a portion of the main fluid line 4.

In order to perform the MCDS, the control unit 24 commands the mixing container outlet valve VMO and the drain valve VDR in the open condition maintaining all the other valves of the fluid circuit 14 closed. In this manner, the control unit 24 sets the mixing container drain path Y1, highlighted in bold in FIG. 1L.

After or simultaneously the opening of the mixing container outlet valve VMO and the drain valve VDR, the control unit 24 runs the pump 8 to determine a suction action in the mixing container 11, the mixing container outlet line 16 and a portion of the main fluid line 4 developing from the first junction point j1 and the pump 8. When the pump 8 is activated by the control unit 24, any liquid and/or substance residual inside the mixing container 11 flows through the outlet 13 to run along the mixing container outlet line 16 through the mixing container outlet valve VMO towards the first junction point j1 to enter the portion of the main fluid line 4 on which the pump 8 is active. The pump 8 pushes this fluid towards the drain outlet line 30 and the drain port 29, through the drain valve 29 to expel the liquid and/or the substances residual from the medical fluid generation apparatus 1.

During the MCDS the control unit 24 operates the pump 8 at pre-set pumping rate and the pre-fixed pressure is equal to or lower than a set pressure value, such as −100 mmHg in case a flexible mixing container 11 is used. Alternatively, in case a rigid mixing container 11 is used, the conductivity meter 25 may be used to detect when air enters it as previously explained.

After the MCDS is performed the control unit 24 runs the MCFS to clean the mixing container 11. In order to run MCFS the control unit 24 commands the valves of the plurality of valves of the fluid circuit 14 to open the water inlet valve WVI and the mixing container inlet valve VMI maintaining all the other valves closed. In this manner, the mixing container filling path Y2, highlighted in bold in FIG. 1M, is set to allow the water arriving from the inlet point 5 to enter the mixing container 11.

After or simultaneously the opening of the water inlet valve WVI and the mixing container inlet valve VMI, the control unit commands the pump 8 at the pre-set pumping rate to pump a volume that is sufficient to achieve proper rinse of the mixing container inlet line 15 and of the mixing container 11.

Once the opening of the water inlet valve WVI and the mixing container inlet valve VMI are switched to the open condition and the pump 8 is activated, the water arriving from the inlet point 5 enters the main fluid line 4 of the fluid circuit 14 passing through the opening of the water inlet valve WVI. The water is transported along the main fluid line 4 passing the first five junction points j1-j5, the heater 23, the conductivity meters 25, 26 and the pump 8. When the water arrives at the sixth junction point j6, it cannot continue towards the common tract 38 of the primary and auxiliary concentrate container inlet 31, 37 as well as the drain outlet line 30 and the port 29 because the concentrate container inlet valves VGI, VAI the drain valve VDR are closed. Therefore, at the sixth junction point j6, the water flows inside the mixing container inlet line 15 towards the mixing container 11. Here, the water cannot reach the medical fluid outlet line 28 and the medical fluid outlet 27 because the medical fluid outlet valve is closed and pass through the mixing container inlet valve VMI to enter into the mixing container 11 through the inlet 12.

Once the water has entered the mixing container 11, it needs to be discharged to clean this latter and the other involved parts of the fluid circuit 14.

After the MCFS, the control unit 24 runs the mixing container emptying step MCES by commanding the valves of the plurality of valves of the fluid circuit 14 to open the mixing container outlet valve VMO and the drain valve VDR, maintaining closed all the other valves of the fluid circuit 14. In this manner, the control unit 24 sets the mixing container emptying path, which is the same of the mixing container drain path Y1 highlighted in bold in FIG. 1L. The mixing container emptying path allows to rinse the mixing container 11, and the mixing container outlet line 16, because the water used for cleaning these parts is wasted out through the drain port 29 after having removed their content.

To achieve the task, in case a flexible mixing container 11 is used, the pump 8 needs to be activated by the control unit 24 at a pre-set pumping rate and a pre-fixed pressure, particularly detected on the mixing outlet line 16, equal to or lower than for example −100 mmHg. Alternatively, in case a rigid mixing container 11 is used, the conductivity meter 25 may be used to detect when air enters it as previously explained.

After the mixing container emptying step MCES, the control unit 24 may run the PSMF of the liquid concentrate mixing procedure LCMP or may start the DCDP if at least one of the concentrate containers 2, 3 houses a dry concentrate to be dissolved/diluted before mixing.

Whether the PSMF is run, the control unit 24 commands the valves of the plurality of valves to open the water inlet valve WVI and the drain valve VDR, maintaining all the other valves, closed. In this manner, the main fluid priming path, which is the same as the main fluid line prime path Z2 highlighted in FIG. 1B, is set to allow the water arriving from the inlet point 5 to fill the main fluid line 4 and to clean the latter.

The control unit 24 also commands the pump 8 at a pre-set pumping rate and a pre-fixed time period to move the water arriving from the inlet point 5 along the main fluid line 4 passing through the water inlet valve WVI, touching the first five junction points j1-j5, the heater 23, the conductivity meters 25, 26, the pump 8 to arrive at the sixth junction point j6. Here, the water may only continue towards the drain outlet line 30 and the drain port 29 because the common tract 38 of the concentrate container inlet lines 31, 37 and the mixing inlet line 15 cannot be reached since the concentrate container inlet valves VGI, VAI, the mixing container inlet valve VMI and the medical fluid outlet valve VFO are closed. The pump 8 is run until conductivity meter 25 indicates a measured conductivity low enough to not significantly impact the composition accuracy in the following steps and after a volume of at least equal or higher than the volume of the main fluid line 4 between first junction point j1 and sixth junction point j6 has been pumped. When the water flows through the heater 23, it is heated up to e.g., 37 °C.

In the place of the PSMF or later than such step, the control unit 24 may run the valves of the plurality of valves 11 to perform a DCDP for any concentrate container 2, 3 housing a dry concentrate DC.

With reference to a situation like the one shown in FIG. 1, where both the primary concentrate container 2 and the auxiliary concentrate container 3 house a corresponding DC to be dissolved, the control unit 24 runs the DCDP (FIG. 5) by sending the water arriving from the inlet point 5 to at least one of the concentrate containers 2, 3 to dissolve the corresponding DC and form the respective concentrated mixture CM.

In order to run the DCDP the control unit 24 runs at least one concentrate mixture recirculation step CMRS (FIG. 5), to allow the corresponding CM to recirculate and homogenize by flowing through the respective concentrate recirculation path Z6 (FIG. 1F) defined into the fluid circuit 14. If necessary, before running the CMRS, the control unit 24 may also run an AES, a MFPS, a RPPS, and a DLFS (FIG. 6).

In particular, the DLFS comprises the control unit 24 running at least two dissolution liquid fill steps DLFS′, DLFS″, namely a first dissolution liquid fill step DLFS' and a second dissolution liquid fill step DLFS″ (FIG. 6). As shown in FIG. 1A, when the control unit 24 runs the AES, the control unit 24 commands the plurality of valves to set the air evacuation flow path Z1 to evacuate air from the primary concentrate container 2.

More in detail, the control unit 24 commands the primary concentrate container outlet valve VGO acting on the primary concentrate container outlet line 32 and the drain valve VDR acting on the drain outlet line 30 to an open condition, while keeping the other valves of the plurality of valves configured to operate on the fluid circuit 14 closed.

After or simultaneously to the opening of the primary concentrate container outlet valve VGO and the drain valve VDR, the control unit 24 activates the pump 8; during AES pumping is performed until low level in the deaeration chamber is ensured. The time to stop condition can be combined with air chamber low level detection (e.g. once low level in chamber is reached plus additional prefixed time amount, e.g., 10s). In the illustrative embodiment for testing purposes, a pre-set pumping rate was about 300 ml/min and a pre-fixed time period was about 20s to remove air from such primary concentrate container 2.

The air inside the primary concentrate container 2 flows out towards the filter 49 and the second primary access 44, then moves towards a first portion 32a of the primary concentrate container outlet line 32 to arrive at the de-aeration chamber 33 filling this latter with air. This is performed to ensure that the volume of air does not contribute to any water dead volume.

The air evacuated from the primary concentrate container 2 is collected into the de-aeration chamber 33 and/or drained out through a second portion 32b of the primary concentrate container outlet line 32 placed downstream the first portion 32a and the de-aeration chamber 33, the primary concentrate container outlet valve VGO, the fifth junction point j5, a portion of the main fluid line 4 where the pump 8 is configured to operate, the sixth junction point j6, the drain outlet line 30, the drain valve VDR, the drain outlet line 30 and the drain port 29.

After the air evacuation step, the control unit 24 runs the priming step of the main fluid line 4 driving the valves of the plurality of valves to set the main fluid line prime path Z2 (FIG. 1B).

In particular, the control unit 24 commands the valves of the plurality of valves to allow a dissolution liquid DL, like water, to enter the main fluid line 4 and flow towards the drain outlet line 30 under the action of the pump 8. More specifically, the control unit 24 commands the water inlet valve VWI placed on the main fluid line 4 and the drain valve VDR acting on the drain outlet line 30 to an open condition, maintaining the other valves of the plurality of valves, different from the water inlet valve VWI and the drain valve VDR, closed.

After or simultaneously to driving the water inlet valve VWI and the drain valve VDR in the open condition, the control unit 24 also commands the pump 8 to prime the main fluid line 4.

During the MFPS, the water arriving from the inlet point 5 enters the main fluid line 4 to pass through the water inlet valve VWI and flows along the main fluid line 4 towards all the junction points j1-j6, the drain valve VDR, the drain outlet line 30, getting out from the drain port 29.

After the MFPS, the control unit 24 runs the RPPS driving the valves of the plurality of valves to set the recirculation priming path Z3 (FIG. 1C). In particular, the control unit 24 commands the primary concentrate container inlet valve VGI acting on the primary concentrate container inlet line 31 and the first concentrate mixing valve VGM acting on the first concentrate mixing line 36 to an open condition, and the other valves of the plurality of valves different from the primary concentrate container inlet valve VGI and the first concentrate mixing valve VGM to a closed condition.

In this manner, the control unit 24 allows a fluid circulation from the sixth junction point j6 on the main fluid line 4 towards the common tract 38, the branch 39, and successively the primary concentrate container inlet line 31. Here, the fluid flows into the primary concentrate container inlet line 31 passing through the primary concentrate container inlet valve VGI and arriving to the corresponding intermediate point 31a where the fluid enters the first concentrate mixing line 36 to run past the first concentrate mixing valve and reach the fourth junction point j4 on the main fluid line 4. Successively, the fluid enters a portion of the main fluid line 4 extending between the fourth junction point j4 and the sixth junction point j6, on which the fifth junction point j5 is placed and the pump 8 is configured to operate, to finally arrive at the sixth junction point j6.

This fluid circulation, from the sixth junction point j6 to the fourth junction point j4 passing through the primary concentrate container inlet line 31 and the first concentrate mixing line 36, has the purpose to move any air present in this tubing section towards the portion of the main fluid line 4 comprised between the fourth junction point j4 and the sixth junction point j6. This is particularly useful for priming the fluid circuit between the sixth junction point j6 and the fourth junction point j4 as well as for directing any air trapped in the recirculation priming path Z3 excluding main fluid line 4 towards the portion of the main fluid line 4 from the fourth junction point j4 to sixth junction point j6. Obviously, the circulating fluid cannot flow over the mixing container inlet valve VMI, the medical fluid outlet valve VFO and the auxiliary concentrate container inlet valve because they are closed.

To perform the fluid circulation in the recirculation priming path Z3, after or simultaneously to driving the primary concentrate container inlet valve VGI and the first concentrate mixing valve VGM to the open condition, the control unit 24 commands the pump 8 according to a pre-set pumping rate and a pre-fixed pumping volume greater than the volume of the portion of the main fluid line 4 from the sixth junction point j6 to the fourth junction point j4 and smaller than the volume of the portion of the main fluid line 4 from the fourth junction point j4 and the sixth junction point j6.

After the recirculation path priming step and the priming of the main fluid path, the control unit 24 commands the valves of the plurality of valves to set the first dissolution liquid fill path Z4 (FIG. 1D) and allow a dissolution liquid DL, like water, to enter the primary concentrate container 2.

In order to do that, the control unit 24 commands the water inlet valve VWI, the primary concentrate container inlet valve VGI on the primary concentrate container inlet line 31 and the gas valve VGA on the vent 34 to an open condition, maintaining the other valves of the plurality of valves different from the water inlet valve VWI, the primary concentrate container inlet valve VGI and the gas valve VGA, closed.

After and/or simultaneously to the opening of the water inlet valve VWI, the primary concentrate container inlet valve VGI and the gas valve VGA, the control unit commands the pump 8, preferably at a pre-set pumping rate and at a pre-fixed pumping volume, to fill a fraction of an expected total dissolution liquid volume in the primary concentrate container 2.

The water entering the main fluid line 4 of the fluid circuit 14 from the inlet point 5 runs along such main fluid line 4 towards the sixth junction point j6, passing through all the others junction points j1-j5 and the heater 23 which is operated by the control unit 24 to heat the water up to at least e.g., 70 °C and optionally up to at least 80 °C.

Since the mixing container inlet valve VMI, the medical fluid outlet valve VFO, the drain valve VDR and the second concentrate container inlet valve VAI are closed, the water arriving from the sixth junction point j6 may only occupy the accessible parts of the mixing container inlet 15 where the pressure sensor P2 is configured to operate and flows through the common tract 38 to arrive at the branch 39 and enter into the primary concentrate container inlet line 31. Here, the water is free to pass through the primary concentrate container inlet valve VGI, which is open, towards the first primary access 43.

In the meantime, the control unit 24 receives pressure data from the pressure sensor P2, in particular a reference pressure value (P2_base), detected in the first dissolution liquid fill path Z4, to be stored in a memory support.

Since also the first concentrate mixing valve VGM is closed, the water running along the primary concentrate container inlet line 31 may only move forward towards the first primary access 43.

After passing the first primary access 43, the water arriving from the primary concentrate container inlet line 31 enters the primary concentrate container 2 through the first port 6 and starts the dissolution of the primary dry concentrate. Once the primary container 2 is completely filled with water and primary concentrate, defining a first liquid mixture ML′, such first liquid mixture ML′ flows out the primary container 2 through the second port 7 towards the second primary access 44. A filling of the primary concentrate container 2 from the bottom enhance the dissolution process, in particular because the water has to pass through the dry concentrate and to remain inside the primary concentrate container 2 until the fluid level reaches the top of the container.

Thus, the first liquid mixture ML′ enters the first portion 32a of the primary concentrate container outlet line 32 and reaches the de-aeration chamber 33. Since the gas valve VGA is open, the air inside the primary container 2 and/or in the first portion 32a of the primary concentrate container line 32 and/or inside the de-aeration chamber 33 may pass out via the vent line 34 as the fluid level rises in the primary container 2 and/or in the de-aeration chamber 33. When the first liquid mixture reaches a pre-set liquid high level in the de-aeration chamber 33, the control unit 24 stops the first dissolution liquid fill step FLDS′.

After the DLFS′, the control unit 24 commands the valves of the plurality of valves to perform the DLFS″, wherein the control unit 24 commands the valves of the plurality of valves to set the second dissolution liquid fill path Z5 (FIG. 1E) to fill up an expected total dissolution liquid volume in the primary concentrate container 2.

More specifically, the control unit 24 commands or maintains the water inlet valve VWI and a primary concentrate container inlet valve VGI in the open condition, maintaining at the same time the other valves of the plurality of valves configured to operate on the fluid circuit 14 different from the water inlet valve VWI and the primary concentrate container inlet valve VGI, closed, including the gas valve VGA in the vent line 34 downstream the de-aeration chamber 33.

After or simultaneously to driving the water inlet valve VWI and the primary concentrate container inlet valve VGI, the control unit 24 commands the pump 8 at a pre-set pumping rate.

Preferably at the same time, the control unit 24 activates the heater 23 on the main fluid line 4 in order to heat the dissolution liquid DL, i.e., water, flowing through the main fluid line 4 up to e.g., at least 70 °C and optionally up to at least 80 °C.

Once the water inlet valve VWI and the primary concentrate container inlet valve VGI are in the open condition and the pump 8 is activated, the water arriving from the inlet point 5 enters the main fluid line 4 passing through the water inlet valve VWI, the first five junction points j1-j5, the heater 23 to arrive at the sixth junction point j6.

Since the mixing container inlet valve VMI, the medical fluid outlet valve VFO, the drain valve VDR and the second concentrate container inlet valve VAI are closed, the water arriving from the sixth junction point j6 may only occupy the accessible parts of the mixing container inlet 15 where the pressure sensor P2 is configured to operate and flows through the common tract 38 to arrive at the branch 39 and enter the primary concentrate container inlet line 31. Here, water is free to pass through the primary concentrate container inlet valve VGI, which is open, to flow towards the first primary access 43.

In the meantime, the control unit 24 receives pressure data detected in the second dissolution liquid fill path Z5 by the pressure sensor P2. Such pressure data are stored in a proper memory support and/or compared with the P2_base detected during the DLFS' in the first dissolution liquid fill path Z4.

Since also the first concentrate mixing valve VGM is closed, the water running along the primary concentrate container inlet line 31 may only move towards the first primary access 43 entering the primary concentrate container 2 through the first port 6 contributing to provide a further dissolution of the primary dry concentrate.

The second dissolution fill step DLFS″ maintains the water filling until a certain overpressure has been built up in the concentrate primary container 2 ensuring that the top outlet corresponding to the second port 7 does not collapse during the following recirculation step and enabling passive de-aeration of air during such recirculation step. The second dissolution fill step is interrupted, by stopping the pump 8, when a pre-fixed pressure in the second dissolution liquid fill path Z5 is reached. In particular, the pressure limit which determines the DLFS″ be interrupted, is defined as the reference pressure value (P2_base) detected in the first dissolution liquid fill path Z4 increased by an overpressure value.

After the DLFS and, in particular after the DLFS″, the control unit 24 commands the plurality of valves to set the first concentrate recirculation path Z6 (FIG. 1F) in order to re-circulate the first liquid mixture ML′ to dissolve the DC and homogenously mix the concentrate liquid mixture obtained.

In particular, the control unit 24 commands, the primary concentrate container outlet valve VGO configured to operate on the primary concentrate container outlet line 32 and the primary concentrate container inlet valve VGI configured to operate on the primary concentrate container inlet line 31, to an open condition, maintaining the other valves of the plurality of valves configured to operate on the fluid circuit 14, different from the primary concentrate container outlet valve VGO and the primary concentrate container inlet valve VGI, closed.

After or simultaneously to driving the primary concentrate container outlet valve VGO and the primary concentrate container inlet valve VGI, the control unit 24 commands the pump 8 at a pre-set pumping and for a pre-fixed time period.

The activation of the pump 8 determines the first liquid mixture ML′ to flow from the primary concentrate container 2 through the second port 7 to the second primary container access 44 entering the first portion 32a of the primary concentrate container outlet line 32. Once inside the primary container outlet line 32, the first liquid mixture ML′ continues to advance towards the de-aeration chamber 33 to flow into the second portion 32b of the primary concentrate container outlet line 32. Here, the first liquid mixture ML′ is free to pass through the primary concentrate container outlet valve VGO which is open to reach the fifth junction point j5 and enter the main fluid line 4 of the fluid circuit 14.

After entering the portion of the main fluid line 4 extending between the fifth junction point j5 and the sixth junction point j6, the first liquid mixture ML′ reaches the heater 23, which has been activated by the control unit 24 to heat the first liquid mixture ML′ up to e.g., at least 50 °C and optionally up to at least 60 °C.

Once heated, the first liquid mixture ML′ flows towards the sixth junction point j6 where it may only enter the common tract 38, because the mixing container inlet valve VMI and the medical fluid outlet valve VFO as well as the drain valve VDR are closed.

When the first liquid mixture ML′ reaches the branch 39, it continues along the primary concentrate container inlet line 31, because the second concentrate container inlet valve VAI is closed, whereas the primary concentrate container inlet valve VGI is open.

The first liquid mixture ML′ cannot flow along the first concentrate mixing line 36 because the first concentrate mixing valve VGM is closed, whereby the first liquid mixture is forced to go ahead towards the first concentrate access 43 and the first port 6 of the primary concentrate container 2 to re-enter inside the container defining the first concentrate recirculation path Z6.

During circulation of the first liquid mixture ML′ along the first concentrate recirculation path Z6, the control unit 24 performs a concentration test step CTS to measure the concentration of the first liquid mixture ML′ and compare the measured concentration with an expected concentration value of such first concentrate liquid. In particular, the CTS may be performed on the first liquid mixture ML′ which flows through the conductivity meters 25, 26, between the heater 23 and the pump 8, configured to operate on the main fluid line 4 of the fluid circuit 14. Sensor SG may be used for the purpose. In case glucose is contained in the primary concentrate container 2 then the sensor SG is a glucose sensor. The CTS may be performed continuously on the first liquid mixture ML′, or according to a preset sampling, to verify the achievement of the desired concentration. Additionally, a test to check whether a complete dissolution occurred may be performed. For example, if conductivity of the first liquid mixture ML′ still increases during the first concentrate recirculation path Z6, the concentrate dissolution is not complete. Vice versa, stability and invariance of conductivity indicate that the dissolution process has completed.

When the auxiliary concentrate container houses a liquid concentrate which does not need to be dissolved according to the DCDP schematically shown in FIGS. 5 and 6 and just aforementioned, after the DLFS″ and the corresponding concentration test step CTS, the control unit 24 may run the other steps of the liquid concentrate mixing procedure LCMP.

Otherwise, whether also the auxiliary concentrate container 3 houses a DC another DCDP very similar to the one performed for the primary dry concentrate housed in the primary concentrate container 2, is run.

As shown in FIG. 4B, it is possible that the fluid circuit 14 is provided with an auxiliary de-aeration chamber 51 connected to the vent line 35 of de-aeration chamber 33 (or to an auxiliary vent line) and having a corresponding auxiliary gas valve VGA2 ready to drain the air contained into the auxiliary container 3. Alternatively, as shown in FIG. 4C, it is possible to provide a fluid circuit 14, able to put the auxiliary container 3 in fluid communication with the de-aeration chamber 33 for draining the undesired air inside the auxiliary concentrate container 3. In this configuration, the auxiliary concentrate container outlet line 40 connects the second auxiliary container access 46 to the primary concentrate container outlet line 32 just upstream the de-aeration chamber 33. Auxiliary concentrate container outlet valve VAO is directly upstream the line connection to the primary concentrate container outlet line 32 (no 3rd junction point j3 to the main fluid line 4 is therefore necessary).

Regardless the configuration provided for draining the air housed inside the auxiliary container 3, the DCDP is substantially the same as the one performed for the primary dry concentrate. Of course, temperatures of the fluids and timing of the steps may change.

In particular, with reference the DCDP of the auxiliary dry concentrate housed inside the auxiliary concentrate container 3, such procedure starts when the control unit 24 runs an AES-Aux, i.e., when the control unit 24 commands the valves of the plurality of valves to set an auxiliary air evacuation flow path X1 (FIG. 1G) in order to evacuate undesired air from the auxiliary concentrate container 3.

More in detail, the control unit 24 commands the auxiliary concentrate container outlet valve VAO acting on the auxiliary concentrate container outlet line 40 and the drain valve VDR acting on the drain outlet line 30 to an open condition, while keeps the other valves of the plurality of valves, different from the auxiliary concentrate container outlet valve VAO and the drain valve VDR, closed.

After or simultaneously to the opening of the auxiliary concentrate container outlet valve VAO and the drain valve VDR, the control unit 24 commands the pump 8, preferably at a pre-set pumping rate and for a pre-fixed time period, to remove air from such auxiliary concentrate container 3. Pumping is performed until low level in the deaeration chamber is ensured.

The air inside the auxiliary concentrate container 3 flows out this latter towards the filter 49 and the second auxiliary access 46 through which it may run along the auxiliary concentrate container outlet line 40 to arrive in an auxiliary de-aeration chamber 51—not shown in FIG. 4B—and fill this latter with air. This is performed to ensure that the volume of air does not contribute to any water dead volume.

The air evacuated from the auxiliary concentrate container 3 is filled and collected into the auxiliary de-aeration chamber and/or drained out through the auxiliary concentrate container outlet line 40, the auxiliary concentrate container outlet valve VAO, the third junction point j3 on the main fluid line 4, a portion of the main fluid line 4 where the pump 8 is active, the sixth junction point j6, the drain outlet line 30, the drain valve VDR configured to operate on the drain outlet line 30 and the drain port 29.

After the AES-Aux, the control unit 24 runs an MFPS-Aux driving the valves of the plurality of valves to set an auxiliary main fluid line prime path which is the same as the main fluid line prime path Z2 shown in FIG. 1B.

Therefore, the MFPS-Aux may be visualized by using the main fluid line prime path Z2 of the MFPS performed in relation to DCDP run for the primary concentrate container 2.

In particular, the control unit 24 commands the valves of the plurality of valves to allow a dissolution liquid, like water, to enter the main fluid line 4 and flow towards the drain outlet line 30 under the action of the pump 8. More specifically, the control unit 24 commands the water inlet valve VWI placed on the main fluid line 4 and the drain valve VDR acting on the drain outlet line 30 to an open condition, maintaining the other valves of the plurality of valves, different from the water inlet valve VWI and the drain valve VDR, closed.

After or simultaneously driving the water inlet valve VWI the drain valve VDR, the control unit 24 also commands the pump 8, until the pre-fixed pumping volume greater than the main fluid line volume from water inlet valve VWI to sixth junction point j6 is achieved, to prime the main fluid line 4.

During the auxiliary priming step of the main fluid line 4, the water arriving from the inlet point 5 enters the main fluid line 4 to pass through the water inlet valve VWI and runs along the main fluid line 4 towards all the junction points j1-j6, the drain valve VDR, the drain outlet line 30, getting out from the drain port 29.

After the auxiliary priming step of the main fluid line MFPS-Aux, the control unit 24 runs an auxiliary recirculation path priming step RPPS-Aux driving the valves of the plurality of valves to set the auxiliary recirculation priming path X2 (FIG. 1H).

In particular, the control unit 24 commands the auxiliary concentrate container inlet valve VAI acting on the auxiliary concentrate container inlet line 37 and the second concentrate mixing valve VAM acting on the second concentrate mixing line 41 to an open condition, and the other valves of the plurality of valves different from the auxiliary concentrate container inlet valve VAI and the second concentrate mixing valve VAM to a closed condition.

In this manner, the control unit 24 allows fluid circulation from the sixth junction point j6 on the main fluid line 4 towards the common tract 38, the branch 39 and successively the auxiliary concentrate container inlet line 37, flowing into the second concentrate mixing line 41 to continue towards the second junction point j2 on the main fluid line 4 and successively into a portion of the main fluid line 4, extending between the second junction point j2 and the sixth junction point j6, on which the third, the fourth and the fifth junction points j3-j5 are placed and the pump 8 is configured to operate, to finally arrive at the sixth junction point j6.

This fluid circulation, from the sixth junction point j6 to the second junction point j2 passing through the auxiliary concentrate container inlet line 37 and the second concentrate mixing line 41, has the purpose to move any air present in this tubing section towards the portion of the main fluid line 4 comprised between the second junction point j2 and the sixth junction point j6. This is particularly useful for priming the fluid circuit between the sixth junction point j6 and the second junction point j2 as well as for directing any air trapped in the auxiliary recirculation priming path X2 (excluding the main fluid line 4 portion) towards the portion of the main fluid line 4 from the second junction point j2 to the sixth junction point j6.

To perform the fluid circulation in the auxiliary recirculation priming path X2, after or simultaneously to driving the auxiliary concentrate container inlet valve VAI and the second concentrate mixing valve VAM to the open condition, the control unit 24 commands the pump 8 according to a pre-set pumping rate and a pre-fixed pumping volume greater than the volume of the portion of the main fluid line 4 from the sixth junction point j6 to the second junction point j2 and smaller than the volume of the portion of the main fluid line 4 from the second junction point j2 and the sixth junction point j6.

After the auxiliary recirculation path priming step and the priming of the main fluid path, the control unit 24 commands the valves of the plurality of valves to run at least one auxiliary dissolution liquid fill step DLFS-Aux, in particular an auxiliary first dissolution liquid fill step DLFS' and an auxiliary dissolution liquid fill step DLFS″.

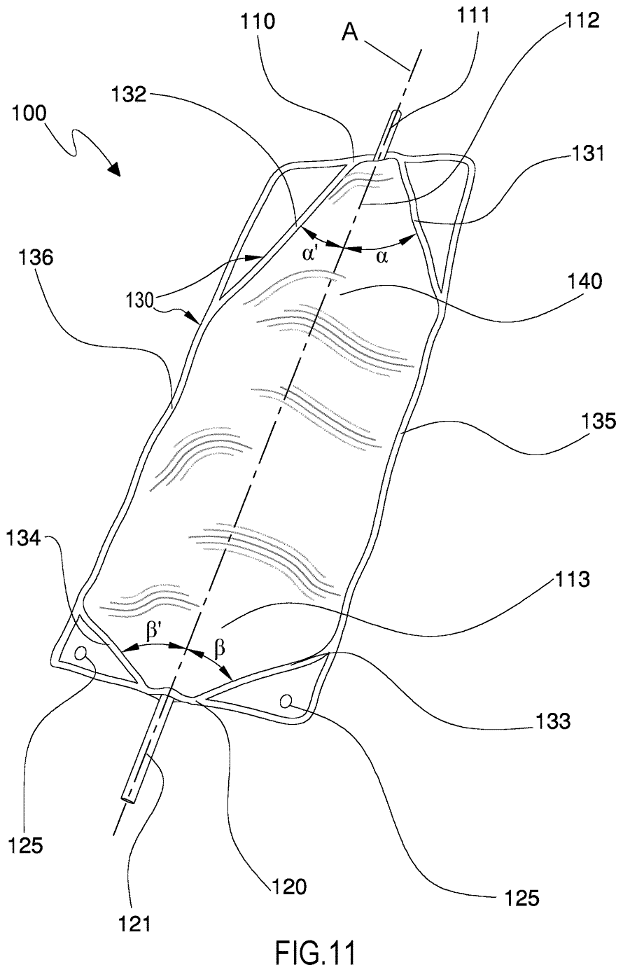

More specifically, during the DLFS' the control unit 24 commands the valves of the plurality of valves to set a first auxiliary dissolution liquid fill path X3 (FIG. 11) and allow a dissolution liquid, like water, to enter the auxiliary concentrate container 3. In order to do that, the control unit 24 commands the water inlet valve VWI, the auxiliary concentrate container inlet valve VAI on the auxiliary concentrate container inlet line 37 and the auxiliary gas valve VGA2 on the auxiliary vent in an open condition, maintaining the other valves of the plurality of valves, closed (see also FIG. 4B).

After and or simultaneously, the control unit commands the pump 8, preferably at a pre-set pumping rate and at a pre-fixed pumping volume, to fill a fraction of an expected total dissolution liquid volume in the auxiliary concentrate container 3.

The water entering the main fluid line 4 of the fluid circuit 14 from the inlet point 5 runs along such main fluid 4 towards the sixth junction point j6, passing through all the others junction points j1-j5 and the heater 23 which may be operated by the control unit 24 to heat the water up to a proper value; the temperature proper value is a temperature value sufficient to achieve efficient dissolution and depends on the substance/s to be dissolved.

Since the mixing container inlet valve VMI, the medical fluid outlet valve VFO, the drain valve VDR and the first concentrate container inlet valve VGI are closed, the water arriving from the sixth junction point j6 may only occupy the accessible parts of the mixing container inlet 15 where the pressure sensor P2 is configured to operate and flows through the common tract 38 to arrive at the branch 39 and enter into the auxiliary concentrate container inlet line 37. Here, the water is free to pass through the auxiliary concentrate container inlet valve VAI, which is open, towards the first auxiliary access 45.

In the meantime, the control unit 24 receives pressure data from the pressure sensor P2, in particular a reference pressure value (P2_base), detected in the first auxiliary dissolution liquid fill path X3, to be stored in a proper memory support.

Since also the second concentrate mixing valve VAM is closed, the water running along the auxiliary concentrate container inlet line 37 may only go ahead towards the first auxiliary access 45. After passing the first auxiliary access 45, the water arriving from the auxiliary concentrate container inlet line 37 enters the auxiliary concentrate container 3 through the first port 9 and starts the dissolution of the auxiliary dry concentrate.

Once the auxiliary container 3 is completely filled with water and the auxiliary concentrate 3a, defining a second liquid mixture ML″, such second liquid mixture ML″ flows out the auxiliary container 3 through the second port 10 towards the second auxiliary access 46. Thus, the second liquid mixture ML″ enters the auxiliary concentrate container outlet line 40 and reaches the corresponding auxiliary de-aeration chamber (see FIG. 4B).

Since the corresponding auxiliary gas valve (not shown) is open, the air inside the auxiliary container 2 and/or in the auxiliary concentrate container outlet line 40 and/or inside the corresponding auxiliary de-aeration chamber (not shown) may pass out via the corresponding auxiliary vent line (not shown) as the fluid level rises in the auxiliary container 3 and/or in the corresponding auxiliary de-aeration chamber (not shown).

When the second liquid mixture ML″ reaches a pre-set liquid high level in the corresponding auxiliary de-aeration chamber (not shown), the control unit 24 stops the DLFS′-Aux.

After the DLFS′-Aux, the control unit 24 may commands again the valves of the plurality of valves to perform at least one more auxiliary dissolution liquid fill step, i.e., the DLFS″-Aux.

In particular, the control unit 24 commands the valves of the plurality of valves to set a second auxiliary dissolution liquid fill path X4 (FIG. 1J) to fill up an expected total dissolution liquid volume in the auxiliary concentrate container 3. More specifically, the control unit 24 commands or maintain the water inlet valve VWI and the auxiliary concentrate container inlet valve VAI in the open condition, maintaining the other valves of the plurality of valves configured to operate on the fluid circuit 14 different from the water inlet valve VWI and the primary concentrate container inlet valve VAI, closed, including the auxiliary gas valve (not shown) in the vent line 34 (not shown).

After or simultaneously to driving water inlet valve VWI and the auxiliary concentrate container inlet valve VAI, the control unit 24 commands the pump 8 at a pre-set pumping rate.

Preferably at the same time, the control unit 24 activates the heater 23 on the main fluid line 4 in order to heat the dissolution liquid, i.e., water, flowing through the main fluid line 4 up to at least 70 °C and optionally up to at least 80 °C.

Once the water inlet valve VWI and the auxiliary concentrate container inlet valve VAI are in the open condition the pump 8 is activated, the water arriving from the inlet point 5 enters the main fluid line 4 passing through the water inlet valve VWI, the first five junction points j1-j5, the heater 23 to arrive at the sixth junction point j6. Since the mixing container inlet valve VMI, the medical fluid outlet valve VFO, the drain valve VDR and the first concentrate container inlet valve VGI are closed, the water arriving from the sixth junction point j6 may only occupy the accessible parts of the mixing container inlet 15 where the pressure sensor P2 is configured to operate, and flows through the common tract 38 to arrive at the branch 39 and enter the auxiliary concentrate container inlet line 37. Here, the water is free to pass through the auxiliary concentrate container inlet valve VAI, which is open, towards the first primary access 45.

In the meantime, the control unit 24 receives, from the pressure sensor P2, pressure data detected in the second auxiliary dissolution liquid fill path X4, to be stored in a proper memory support and/or compared with the reference pressure data detected during DLFS′-Aux in the first auxiliary dissolution liquid fill path X3.

Since also the second concentrate mixing valve VAM is closed, the water running along the primary concentrate container inlet line 31 may only go ahead towards the first auxiliary access 45 entering the auxiliary concentrate container 3 through the first port 9 and contributing to further dissolve the auxiliary dry concentrate. The DLFS″-Aux maintains the water filling until a certain overpressure has been built up in the concentrate auxiliary container 3 ensuring that the top outlet corresponding to the second port 10 does not collapse during the following auxiliary recirculation step and enabling passive de-aeration of air during such auxiliary recirculation step.

The DLFS″-Aux is interrupted, by stopping the pump 8, when a pre-fixed pressure in the second auxiliary dissolution liquid fill path X4 is reached.

In particular, the pressure limit which determines the second auxiliary dissolution liquid fill path is interrupted, is defined as the reference pressure value (P2_base), detected in the first auxiliary dissolution liquid fill path X3 increased for example by 40 mmHg.

After the DLFS″, the control unit 24 commands the plurality of valves to set the second concentrate recirculation path X5 (FIG. 1K) to re-circulate the second liquid mixture ML″ to dissolve the auxiliary dry concentrate and homogenously mix the second liquid mixture ML″ obtained. In particular, the control unit 24 commands, the auxiliary concentrate container outlet valve VAO configured to operate on the auxiliary concentrate container outlet line 40 and the auxiliary concentrate container inlet valve VAI configured to operate on the auxiliary concentrate container inlet line 37 to an open condition, maintaining the other valves of the plurality of valves configured to operate on the fluid circuit 14, different from the auxiliary concentrate container outlet valve VAO and the auxiliary concentrate container inlet valve VAI, closed.

After or simultaneously to driving the auxiliary concentrate container outlet valve VAO and the auxiliary concentrate container inlet valve VAI, the control unit 24 commands the pump 8 at a pre-set pumping rate and for a pre-fixed time period.

The activation of the pump 8 determines the first liquid mixture ML′ to flow from the auxiliary concentrate container 3 through the second port 10 to the second auxiliary container access 46 entering the auxiliary concentrate container outlet line 40. Once inside the auxiliary container outlet line 40, the second liquid mixture ML″ continues to advance towards the corresponding de-aeration chamber (not shown) to flow towards the third junction point j3. The second liquid mixture ML″ is free to pass through the auxiliary concentrate container outlet valve VAO which is open to reach the third junction point j3 and enter the main fluid line 4 of the fluid circuit 14.

After entering the portion of the main fluid line 4 extending between the third junction point j3 and the sixth junction point j6, the second liquid mixture ML″ reaches the heater 23, which has been activated by the control unit 24 to heat the second liquid mixture ML″ up to at least 50 °C and optionally up to at least 60 °C.

Once heated, the second liquid mixture ML″ flows towards the sixth junction point j6 where it may only enter the common tract 38, because the mixing container inlet valve VMI and the medical fluid outlet valve VFO as well as the drain valve VDR are closed.

When the second liquid mixture ML″ reaches the branch 39 it continues along the auxiliary concentrate container inlet line 37, because the first concentrate container inlet valve VGI is closed, whereas the auxiliary concentrate container inlet valve VAI is open.

The second liquid mixture cannot move along the second concentrate mixing line 41 because the second concentrate mixing valve VAM is closed, whereby the second liquid mixture ML″ is forced to flow towards the first concentrate access 45 and the first port 9 of the auxiliary concentrate container 3 to re-enter inside the same container defining the second concentrate recirculation path X5.

During circulation of the second liquid mixture along the second concentrate recirculation path X5, the control unit 24 performs an auxiliary concentration test step CTS-Aux in order to measure the concentration of the second liquid mixture ML″ and compare the measured concentration with an expected concentration value of such second concentrate liquid. A conductivity meter with e.g., a sufficient measurement range is used. In particular, the auxiliary concentration test step CTS-Aux may be performed on the second liquid mixture ML″ which flows through the aforementioned conductivity meters 25, 26, between the heater 23 and the pump 8, configured to operate on the main fluid line 4 of the fluid circuit 14. The auxiliary concentration test step CTS-Aux may be performed continuously on the second liquid mixture, or according to a preset sampling, in order to verify the achievement of the desired concentration.

For the purposes of the present invention, either carrying out the DCDP in relation to the primary dry concentrate or carrying out the DCDP in relation to the auxiliary dry concentrate 3 first, is substantially indifferent as only some settings differ.

Every DCDP is performed to transform a DC into a diluted concentrate liquid mixture ML ready to be subjected to a mixing operation with water and another liquid mixture.

The DCDP may be performed in the same way when a DC is provided inside a corresponding concentrate container 2, 3 as in the embodiments shown in FIGS. 2 and 3. In particular, considering the embodiment shown in FIG. 2, both the primary concentrate container 2 and the auxiliary concentrate container 3 house dry concentrates. Therefore, the mentioned DCDP, disclosed with the help of the fluid circuit parts highlighted in FIGS. 1A-1K with reference to the embodiment shown in claim 1, may be performed in the same way with reference to the embodiment shown in FIG. 2.

Moreover, all the steps defining the DCDP performed with reference to the primary concentrate housed in the primary container 2 and explained with reference to the first embodiment by using FIGS. 1A to 1F, are substantially the same (note that some settings may change) for the DCDP which may be performed in relation to the primary concentrate housed in the primary container 2 of the apparatus 1 according to the embodiment of FIG. 3.

In order to separate the DCDP performed for the dry primary concentrate housed in the primary concentrate container 2 from the DCDP performed on the dry auxiliary concentrate housed in the auxiliary container 3, a priming step of the main fluid line like the MFPS executed during the DCDP or the PSMF executed during the liquid concentrate mixing procedure LCMP, may be performed.

A priming step of the main fluid line may also be executed to separate the last DCDP just ended and the steps of the liquid concentrate mixing procedure LCMP which has to be run for performing the mixing of the diluted mixtures DM and/or the liquid mixtures LM obtained through the corresponding DCDP.

When the concentrate containers 2, 3 house corresponding liquid concentrates obtained after the corresponding DCDP or liquid concentrates already provided inside them, and after the initial drain, fill and rinse and further emptying of the mixing container and rinse of the main fluid line is performed, the liquid concentrate mixing procedure LCMP continues as follows.

The control unit 24 runs the FL′ of an auxiliary liquid concentrate LC′ housed inside one of the concentrate containers 2, 3 into the mixing container 11. The control unit 24 may perform the FL′ starting from the primary concentrate container 2 or the auxiliary concentrate container 3. It is preferred that the control unit 24 starts the FL′ from the concentrate container 2, 3 housing a liquid concentrate comprising at least electrolyte and buffer.

Supposing that electrolyte and buffer constitutes the auxiliary liquid concentrate LC′ which is housed inside the auxiliary concentrate container 3, the control unit 24 commands the valves of the plurality of valves to set the auxiliary concentrate filling path F1 (FIG. 1N) to fill the mixing container 11 with the auxiliary liquid concentrate LC, particularly according to a pre-fixed nominal volume.

The control unit 24 commands the second concentrate mixing valve VAM configured to operate on the second concentrate mixing line 41 and the mixing container inlet valve VMI configured to operate on the mixing inlet line 15 in the open condition and maintain all the other valves of the plurality of valves closed. In this manner the auxiliary liquid concentrate LC′ arriving from the auxiliary concentrate container 3 is free to move towards the mixing container 11.

After or simultaneously to the opening of the second concentrate mixing valve VAM and the mixing container inlet valve VMI, the control unit 24 commands the pump 8, at the pre-set pumping rate being about 220 ml/min and the pre-fixed pumping volume equal to or higher than the pre-fixed nominal volume of the auxiliary liquid concentrate LC, to move the latter from the auxiliary concentrate container 3 towards the mixing container 11.

In particular, during the first filling step FS′, the auxiliary liquid concentrate LC′ flows out the auxiliary concentrate container 3 through the first port 9 towards the first auxiliary access 45. The auxiliary liquid concentrate LC′ enters the second concentrate container inlet line 37 and the second concentrate mixing line 41 through the first auxiliary access 45 to flow towards the second junction point j2 from which it may enter a portion of the main fluid line extending between the second junction portion j2 and the sixth junction point j6.

Once arrived in the main fluid line 4, the auxiliary liquid concentrate LC′ continues its own path reaching the third junction point j3, the fourth junction points J4, the fifth junction point J5 and the heater 23 where it is heated up to 37 °C. Once the heater 23 has been passed, the auxiliary liquid concentrate LC′ reaches, the conductivity meters 25, 26, passing over the pump 8 to arrive at the sixth junction j6. Since the concentrate container inlet valves VGI, VAI, as well as, the drain valve VDR and medical fluid outlet valve VFO are closed, the auxiliary liquid concentrate LC′ may only move along the mixing container inlet line 15. The auxiliary liquid concentrate LC′ passes through the mixing container inlet valve VMI and the inlet 12 entering into the mixing container 11.

After the first filling step FS' of the first diluted mixture DM′ into the mixing container 11, the control unit 24 runs one filling step of water FSW. In particular, the control unit 24 commands the valves of the plurality of valves to set the water filling path W1 (FIG. 1O) allowing to fill the mixing container 11, at least partially, with a pre-fixed volume of water. In order to perform the water filling step FSW, the control unit 24 commands the water inlet valve VWI configured to operate on the fluid main line 4 and the mixing container inlet valve VMI configured to operate on the mixing inlet line 15 in the open condition and maintain all the other valves of the fluid circuit 14, closed.

After or simultaneously to the opening of the water inlet valve VWI and the mixing container inlet valve VMI, the control unit commands the pump 8, particularly at the pre-set pumping rate and the pre-fixed pumping volume of at least 80% of a calculated reference volume of water already mentioned, to move the water arriving from the inlet point 5 of the fluid circuit 14 towards the mixing container 11. In particular, the water arriving from the inlet point 5 enters the main fluid line 4 passing through the water inlet valve VWI. The water runs along the main fluid line 4 passing the first five junction points j1-j5 and arriving at the heater 23 where it is heated up to 37 °C. Once the heater has been reached, the water pass over the conductivity meters 25, 26 and the pump 8 to arrive at the sixth junction point j6. Here, the water cannot flow towards any concentrate container inlet line 31, 37 and the drain outlet line 30 being the concentrate container inlet valves VGI, VAI as well as the drain valve VDR closed. The water may only move along the mixing container inlet line 15 passing the medical fluid outlet line 28 which is closed by the corresponding medical fluid outlet valve VFO. The water pass through the mixing container inlet valve VMI and the inlet 12 entering the mixing container 11 by diluting the auxiliary liquid concentrate LC′, previously delivered to the mixing container 11.

After performing the first filling step of water FSW, the control unit 24 runs the RS' of the first diluted liquid mixture DM′ (auxiliary liquid concentrate LC′ added with water). In order to run such first filling step of water FSW, the control unit 24 commands the valves of the plurality of valves to set the mixing recirculation path R1 (FIG. 1P) to mix the first diluted mixture DM′ previously filled into the mixing container 11.

In particular, the control unit 24 commands the mixing container outlet valve VMO configured to operate on the mixing outlet line 16 and the mixing container inlet valve VMI configured to operate on the mixing inlet line 15 the open condition and closes all the other valves. Moreover, the control unit 24 commands the pump 8 at the pre-set pumping rate of e.g., about 330 ml/min and the pre-fixed time period of e.g., 60s.

Preferably, during the first recirculation step RS′, the control unit 24 performs the further main fluid line concentration test step ML-CTS′. In particular, the control unit 24 detects, preferably continuously, by means of at least one of the conductivity meters 25, 26 placed between the heater 23 and the pump 8, the conductivity variances of the first diluted mixture DM′ which flows through the mixing recirculation path R1. In detail, the control unit 24 provides at least one comparison between the conductivity variances detected in the first diluted mixture DM′ flowing through the mixing recirculation path R1 and a pre-fixed threshold. When the result of such a comparison is under the pre-fixed threshold, the solution is homogeneously mixed and a signal of interruption of the RS' is emitted and managed by the control unit 24, preferably to run the successive step, for instance the RS″ of the primary liquid concentrate LC″ housed in the primary concentrate container 2 into the mixing container 11. In case the result of the comparison between the conductivity variances detected in the first diluted mixture DM′ flowing through the mixing recirculation path R1 and the pre-fixed threshold is above the pre-fixed threshold, the filling step of water FSW, the RS' and the further main fluid line concentration test step are repeated to bring the conductivity variances detected below the pre-fixed threshold.

Once the conductivity variance(s) detected is/are below the pre-fixed threshold, the control unit 24 first recalculates residual water volume to add and primary concentrate container volume (e.g., glucose concentrate) to add. Then the residual water volume is added and a subsequent recirculation step is performed similar to the first recirculation step above described. The conductivity measured during the subsequent recirculation (with full water amount) indicates whether the correct electrolyte composition was reached. Once the conductivity variance(s) detected in the subsequent recirculation step is/are below the pre-fixed threshold, the control unit starts the second filling step RS″ of the primary liquid concentrate LC″ housed in the primary concentrate container 2, into the mixing container 11. In particular, the control unit 24 commands the valves of the plurality of valves to set the primary concentrate filling path F2 (FIG. 1Q) to fill the mixing container 11 with the primary liquid concentrate LC″ arriving from the primary concentrate container 2. More in particular, the control unit 24 commands the first concentrate mixing valve VGM and the mixing container inlet valve VMI in an open condition and maintains all the other valves of the fluid circuit 14 closed. Later or simultaneously the opening of the first concentrate mixing valve VGM and the mixing container inlet valve VMI, the control unit 24 commands the pump 8, optionally at the pre-set pumping rate of about 200 ml/min and at the pre-fixed pumping volume equal to or higher than a recalculated reference volume of the primary liquid concentrate LC″, as already mentioned in the description.

During the second filling step FS″, the primary liquid concentrate LC″ flows out the primary concentrate container 2 through the first port 6 towards the first primary access 43. The primary liquid concentrate LC″ enters the primary concentrate container inlet line 31 through the first primary access 43 to flow towards the fourth junction point j4 from which it may enter into a portion of the main fluid line 4 extending between the fourth junction portion j4 and the sixth junction point j6.

The primary liquid concentrate LC″ continues along the main fluid line 4 reaching the heater 23 where it is heated up to 37 °C.

After the heater 23, the primary liquid concentrate LC″ reaches the conductivity meters 25, 26, passes via the pump 8 and arrives at the sixth junction j6. Since the concentrate container inlet valves VGI, VAI, as well as, the drain valve VDR and the medical fluid outlet valve VFO are closed, the primary liquid concentrate LC″ may only flow along the mixing container inlet line 15. The primary liquid concentrate LC″ passes through the mixing container inlet valve VMI and the inlet 12 entering the mixing container 11 and joining the first diluted mixture DM′ to form the dialysis mixture DiaM.

After the second filling step FS″ of the primary liquid concentrate LC″ into the mixing container 11, the control unit runs the RS″ of the dialysis mixture DiaM formed by adding the primary liquid concentrate LC″ to the first diluted liquid mixture DM′. In order to perform the RS″ of the dialysis mixture DiaM, the control unit 24 commands the valves of the plurality of valves to set the mixing recirculation path, which is identical to the mixing recirculation path R1 (FIG. 1P) so that the dialysis mixture DiaM contained in the mixing container 11 is properly mixed. The second recirculation path is set by the control unit 24 which commands the mixing container outlet valve VMO and the mixing container inlet valve VMI in the open condition and maintains all the other valves closed.

During the RS″ the control unit 24 commands the pump 8 at the pre-set pumping rate of e.g., about 330 ml/min and for the pre-fixed time period of e.g., 60s to flow the dialysis mixture DiaM through the mixing recirculation path. Further, the heater 23 heats the dialysis mixture DiaM up to e.g., 37 °C.

In order to verify that the dialysis mixture DiaM circulating in the mixing recirculation path has been correctly homogenized for the desired dialysis fluid FD to be prepared, an additional main fluid line concentration test step MF-CTS″ is performed during the RS″. In particular, the additional main fluid line concentration test step MF-CTS″ allows to detect, by means of at least one conductivity meter 25, 26, preferably continuously, conductivity variances of the dialysis mixture DiaM flowing through the mixing recirculation path. The control unit 24 executes comparisons of the conductivity variances detected in the dialysis mixture DiaM with a pre-fixed threshold; a signal of interruption of the second recirculation step of the dialysis fluid is emitted when variance is below the threshold. In this situation, the dialysis mixture DiaM has been transformed into the desired dialysis fluid FD ready to be used in a dialysis treatment.

Once the dialysis fluid FD has been obtained, the control unit 24 controls the valves of the plurality of valves to provide the delivery of the dialysis fluid FD to an external dialysis medical treatment apparatus through the medical fluid outlet line 28 and the medical fluid outlet 29. In order to do that, the control unit 24 commands the medical fluid outlet valve VFO and the mixing container outlet valve VMO in the open condition and maintains all the other valves closed. The activation of the pump 8 determines the dialysis fluid to flow towards the medical fluid outlet line 28 and the medical fluid outlet 29.

With reference to the embodiments shown in FIGS. 2, 3 and 4, the mixing container 11 is placed outside the supporting structure 42. Therefore, the entrance and the exit of the fluids/liquids into and from the mixing container 11 must be performed through the inlet port 47 and the exit port 48.

Finally, FIG. 4A shows a simplified embodiment of the fluid circuit 14 designed for managing liquid concentrates in both primary and auxiliary concentrate containers 2, 3. In such a situation, no dry concentrate dissolution step is required and therefore a number of lines are not strictly necessary. In the shown embodiment, primary concentrate container outlet line 32 and auxiliary concentrate container outlet line 40 are removed. Only a single access for each container 2, 3 is necessary and therefore, first primary container access 43 and first auxiliary container access 45 are present, while second primary container access 44 and second auxiliary container access 46 are omitted. Also third and fifth junction point j3 and j5 are removed.

First concentrate mixing line 36 and second concentrate mixing line 41 respectively connect the primary and auxiliary concentrate containers 2, 3 to the main fluid line 4 at junction points j2 and j4. Respective valves VAM and VGM are placed on the mixing lines 36 and 41, but no branches emerge from the mentioned first concentrate mixing line 36 and second concentrate mixing line 41 as it is the case in FIG. 1. The liquid concentrate mixing procedure remains the same as previously discussed for FIGS. 1N to 1Q. First concentrate mixing line 36 is used in the primary concentrate filling path F2 of FIG. 1Q and second concentrate mixing line 41 is used in the auxiliary concentrate filling path F1 of FIG. 1N.

In some possible alternatives, after flowing the auxiliary liquid concentrate LC′ and the proper amount of water to the mixing container 11 to form the diluted liquid mixture, as indicated, the liquid concentrate mixing procedure provides a repetition of the filling step of water. In this situation, the repetition of the RS' may be omitted. In this case, the second filling step FS″ of the primary liquid concentrate LC″ may be performed before the repetition of the filling step of water.

In a further alternative, the second filling step FS″ of the primary liquid concentrate LC″ (glucose liquid concentrate) may be performed before the RS' (and even before the filling step of water FSW) to let the glucose be present in the electrolyte concentration test volume. This could help to mitigate the potential issue of time consuming mixing of glucose concentrate with the rest of the batch.

Another alternative is to perform a separate electrolyte concentrate test similar to the glucose test after glucose dissolution. The electrolyte concentrate used in the test will then be discarded. The overall waste of electrolyte concentrate will however likely not increase since it is needed in the glucose concentrate test. Concentration test step CTS in the dry concentrate dissolution sequence will hence be a combined glucose plus electrolyte concentrate concentration test from which the known actual concentrations can enable a simpler mixing process wherein all three fluids are dosed volumetrically and then mixed in only one recirculation step followed by a conductivity check. This would save much time which is particularly relevant for first batch since it determines time to treatment.

An alternative to wasting the residual PD fluid volume after fill and perform rinse/drain is to keep the residual PD fluid volume in the mixing container (including dead volumes, if applicable) and use the residual PD fluid volume as a starting point for the subsequent batch mixing. The target conductivities then need to be calculated based on the residual PD fluid volume and composition and the target volume and composition of the next batch to mix. If the residual volume is assumed to be “somewhat unknown” (e.g., due to drift), it is possible to start the next preparation phase with an “inverted” concentration test, i.e., add some water and from homogenous conductivity before and after adding water the residual volume is easily calculated.

### Dry Concentrate Bag

Usually, at least one of the primary concentrate container 2 or the auxiliary concentrate container 3 is in the form of a bag, particularly housing a dry concentrate to be diluted. Both bags may house dry concentrate to be diluted and may assume any of the shapes described herein after.

Some preferred, but not limiting, embodiments of the dry concentrate bag are schematically shown in figures from 10 to 21, wherein reference number 100 is directed to a bag containing a dry concentrate 104, for example a powder or granulate.

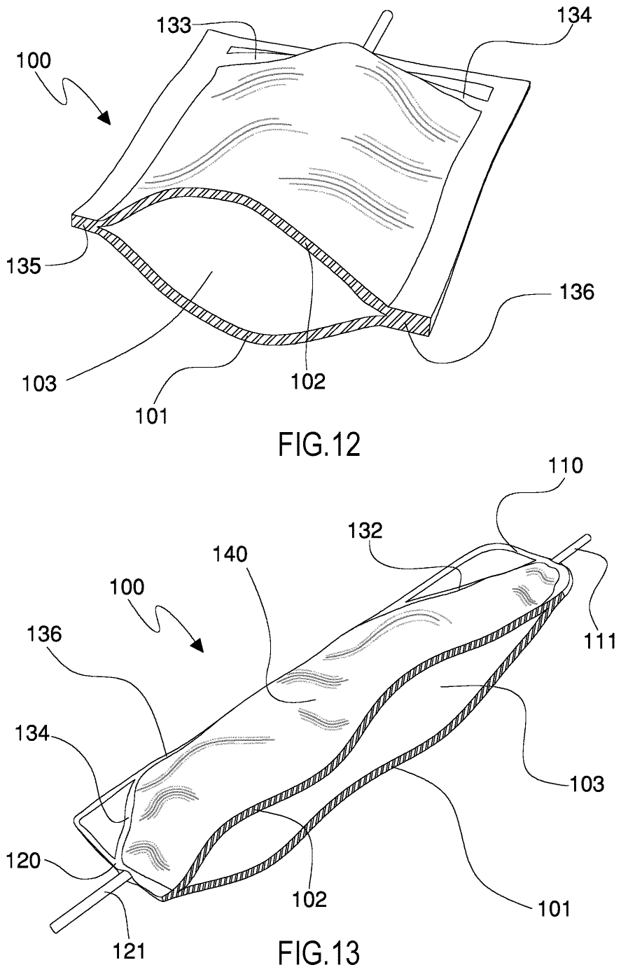

The dry concentrate bag 100 may comprise two plastic films 101, 102 facing each other and welded together to define an internal housing space 103 for containing the dry concentrate 104 (see e.g., FIG. 12). The plastic film may be multilayer film, for example made up of 3 to 7 layers. The thickness of the multi-layer film is not per se particularly critical. It is, however, preferred to be at least 100 μm thick and less than 200 μm. The film may be made of medical grade polyethylene or medical grade PVC. In particular, the film is transparent and flexible, but also heat-sterilizable, fusible and heat sealable. The two plastic films may have a square or rectangular shape. Notably, before welding, the two plastic films may be distinct (i.e., separate) one from the other.

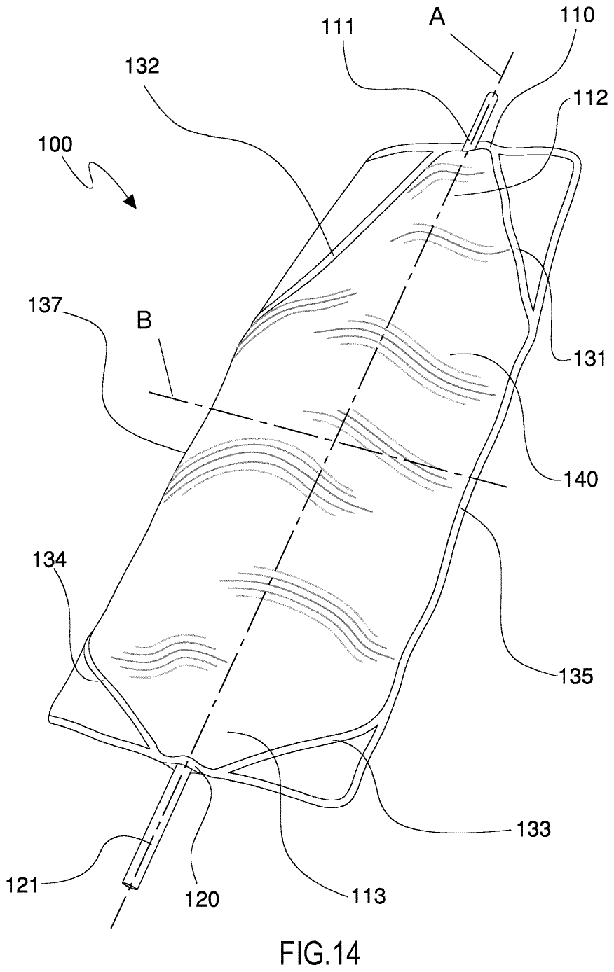

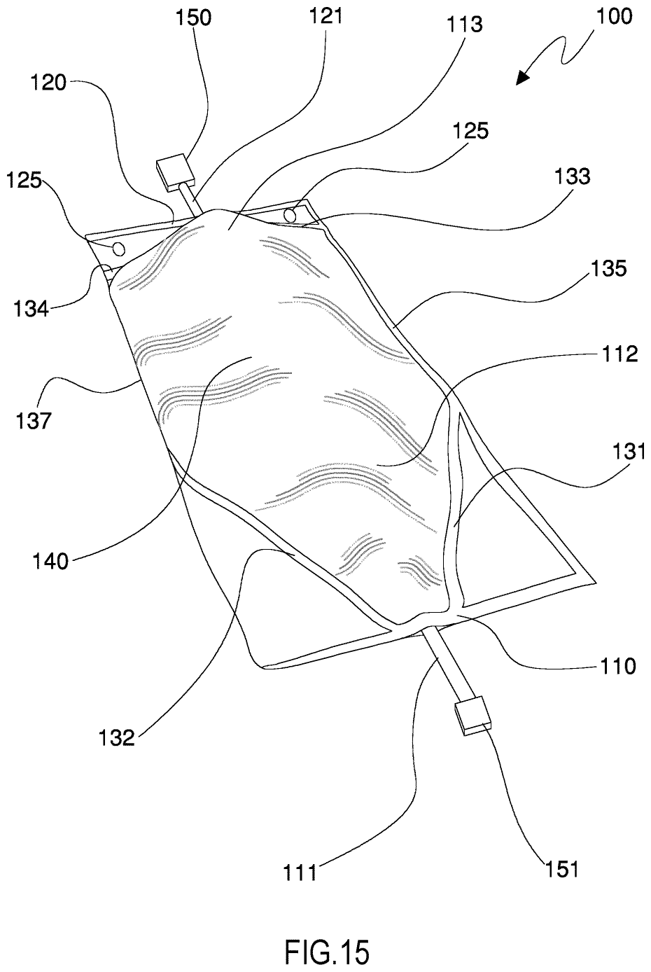

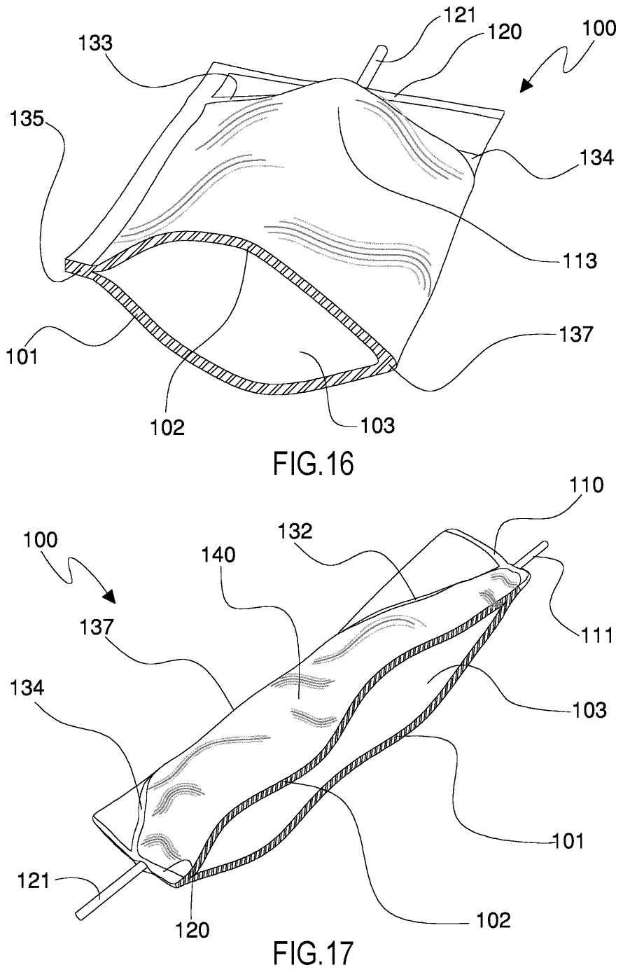

According to a further embodiment (FIGS. 14 to 17), the dry concentrate bag 100 may comprise a single plastic film folded back on itself so that a first layer 101 of the plastic film is faced to a second layer 102 of the plastic film: the first and the second layers 101, 102 are welded together to define the internal housing space 103 for containing the dry concentrate 104. The single plastic film may have a thickness comprised between 100 μm and 200 μm and may comprise medical PVC: the plastic film may be made of medical grade polyethylene or medical grade PVC. The single plastic film may have a square or rectangular shape.

The plastic material of the films and the dry concentrate bag 100 should preferably withstand a temperature of at least 80 °C.

According to the embodiments comprising two plastic films or the single plastic film, the dry concentrate bag is flexible, so that the housing space 103 is configured to increase a volume thereof between a condition wherein the housing space 103 is empty, and a condition wherein the housing space 103 houses the dry concentrate 104 and/or a medical fluid. In particular, when the housing space 103 is empty, namely when no dry concentrate and no fluid is housed in the bag, the dry concentrate bag is substantially flat. On the contrary, when the housing space 103 is filled with dry concentrate, the dry concentrate bag expands in volume, defining substantially a tridimensional shape.

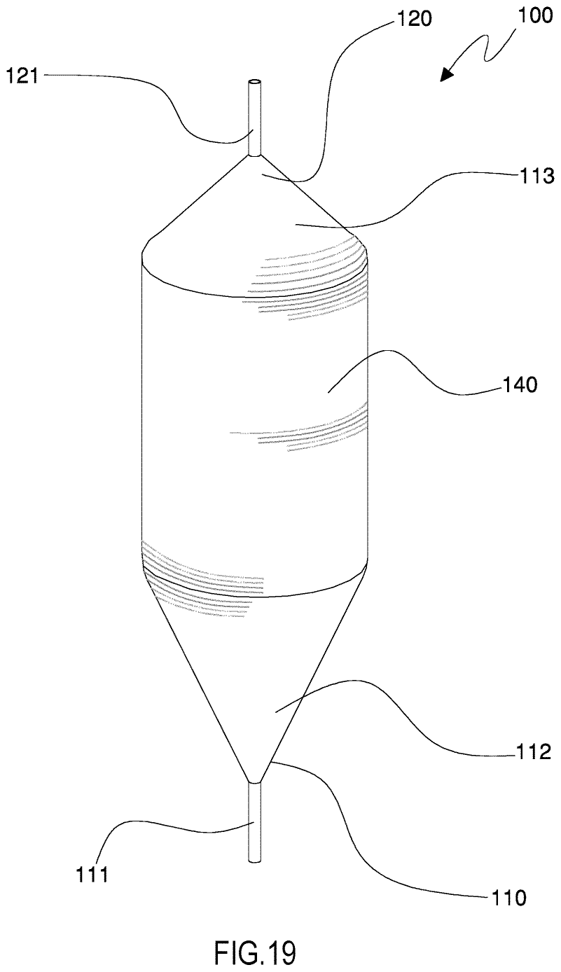

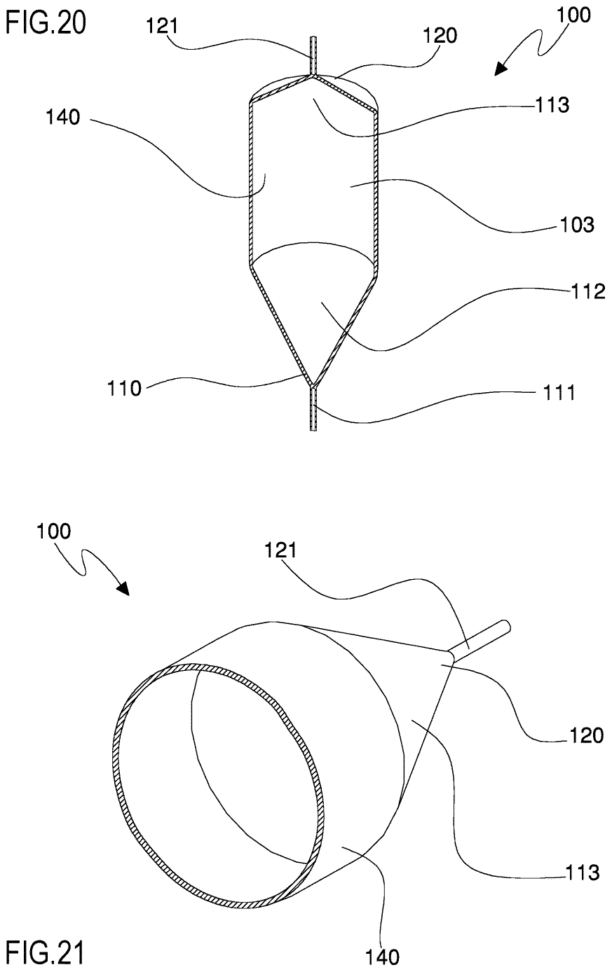

According to an optional embodiment (see FIGS. 19 to 21), the dry concentrate bag 100 may comprise a body made of plastic having a stiffness higher than a corresponding stiffness of the plastic film of the preceding embodiments so that the tridimensional shape of the bag may be maintained (see FIGS. 19-21). According to a further alternative embodiment, the dry concentrate bag 100 may comprise a body made of glass. Notably, according to these last embodiments wherein the body is made of more rigid plastic or glass, the housing space 103 defines a substantially predefined and not-variable internal volume: in other terms, the housing space 103 does not substantially vary the volume thereof between a condition wherein the bag is filled with the dry concentrate, and a condition wherein the bag is empty. The above materials to make the dry concentrate bag are mentioned as exemplary embodiments: however, the bag may be made by further materials compatible with medical fluids and medical dry substances.

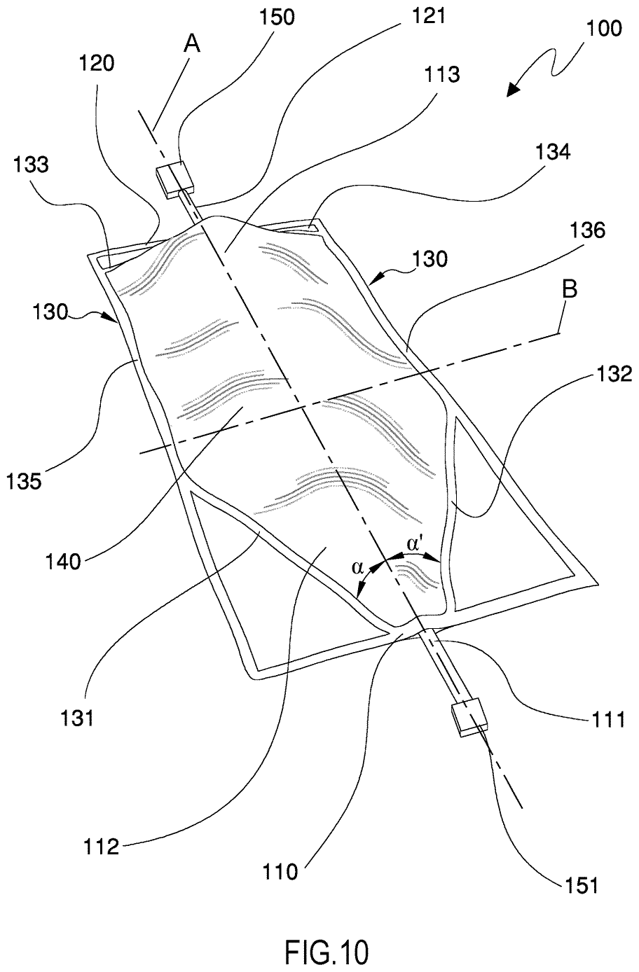

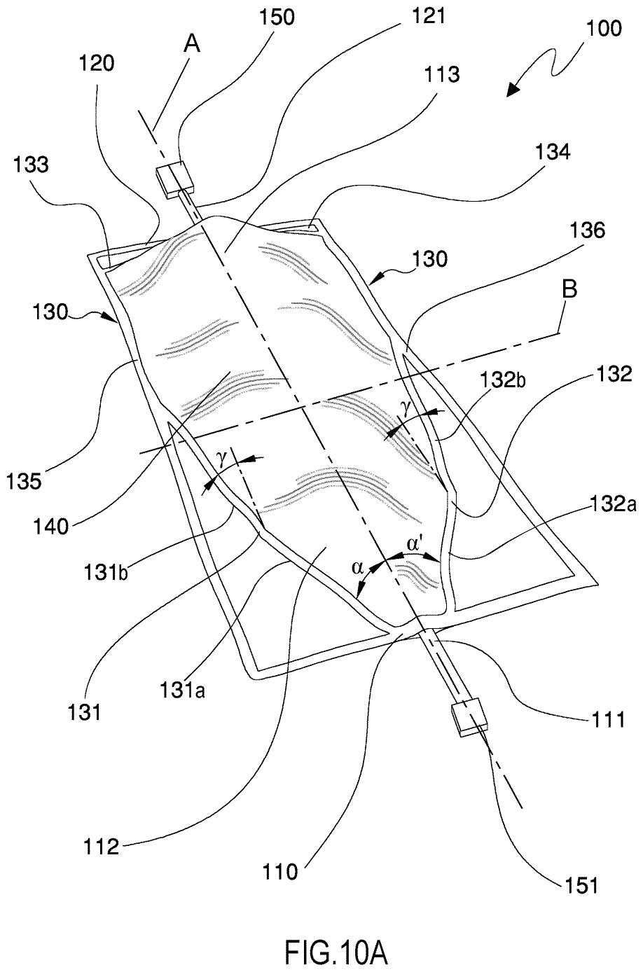

Beyond the materials mentioned above, looking for example to FIG. 10, the dry concentrate bag 100 comprises an inlet portion 110 comprising an inlet port 111 for receiving a medical fluid: the inlet port 111 is placed in correspondence of a bottom area, in use conditions, of the dry concentrate bag 100. The dry concentrate bag 100 further comprises an outlet portion 120 comprising an outlet port 121 for letting the fluid mixed with the dry concentrate 104 to leave the housing space 103: the outlet port 121 is placed in correspondence of a top area in use conditions of the dry concentrate bag 100. The medical fluid entering from the inlet port may comprise water, in particular pure water; alternatively (for example in a recirculation condition) the medical fluid entering from the inlet port may comprise a diluted concentrate or a dialysis fluid.

The housing space 103 at the inlet portion 110 (see also FIG. 13) defines a lower cone portion 112, wherein the inlet port 111 is placed at the lowest point of the lower cone portion 112. The lower cone portion 112 is “cone” or “V” shaped, wherein the minor base of the cone is located at the inlet port 111, and the major base of the cone is distanced from the inlet port 111. Optionally the major base of the lower cone is substantially located at a central/middle portion of the dry concentrate bag 100. In particular, the housing space 103 at the lower cone portion 112 is “V” shaped in a condition wherein the housing space 103 is substantially empty, while the housing space 103 at the lower cone portion 112 is “cone” shaped in a condition wherein the housing space 103 is filled with the dry concentrate or with fluid. Anyhow, the term “cone” used in the present description and claims is directed to globally cover both the “V′ and the “cone” shape.

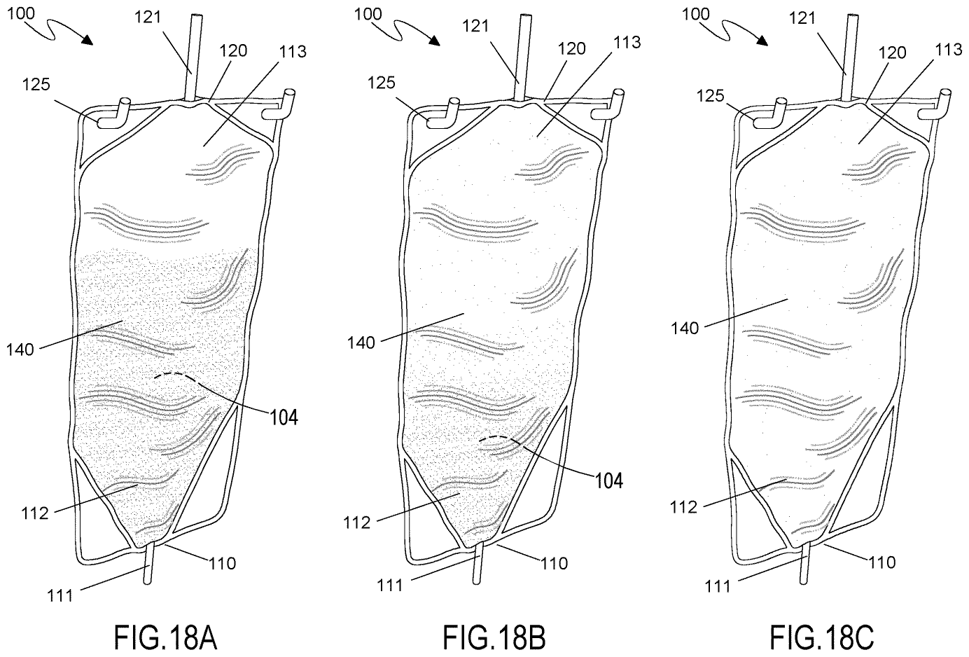

Analogously, the housing space 103 at the outlet portion 120 defines an upper cone portion 113: the outlet port 121 is placed at the highest point of the upper cone portion 113. In particular the upper cone portion 113 is “cone” or “V” shaped, wherein the minor base of the cone is located the outlet port 121, and the major base of the cone is distanced from the outlet port 121. Optionally the major base of the upper cone is substantially located at a central/middle portion of the dry concentrate bag 100: optionally the major base of the lower cone and the major base of the upper cone may coincide: alternatively a central portion 140 (FIGS. 18A-18C) may be located between the upper cone portion 113 and the lower cone portion 112. In particular, the housing space 103 at the upper cone portion 113 is “V” shaped in a condition wherein the housing space 103 is substantially empty, while the housing space 103 at the upper cone portion 113 is “cone” shaped in a condition wherein the housing space 103 is filled with the dry concentrate or with fluid. Anyhow, as already specified above, the term “cone” used in the present description and claims is directed to globally cover both the “V” and the “cone” shape.

Notably, the housing space 103 extends in length along a longitudinal axis A passing through the inlet port 111 and the outlet port 121, and extends in width along a transversal axis B orthogonal to the longitudinal axis A.

Starting from the inlet port 111, the width of the housing space 103 increases with the length up to a maximum width of the housing space 103. In particular the maximum width corresponds to the major base of the lower cone portion 112. Furthermore, starting from said maximum width, the width of the housing space 103 decreases with the length up to the outlet port 121. In particular, this increasing and decreasing in width of the housing space 103 is monotonic with the length. The bag 100 may also comprise a constant tract wherein the width of the housing space 103 is substantially constant: the constant tract is located between the increasing and the decreasing in width. The trend of the width of the housing space 103 is shown in FIG. 14.

The lower cone portion 112 does not include dead volumes near the inlet port 111: analogously, the upper cone portion 113 does not include dead volumes near the outlet port 121. Thus, the housing space 103 does not include dead volumes. The expression “dead volume” may be defined as a volume of the housing space that is not easily fillable by the fluid and/or by the dry concentrate: in other terms, the dead volume is a portion of the housing space that cannot easily be reached by the fluid and/or by the dry concentrate. Thus, the whole volume defined by the housing space 103 is reachable by the fluid and/or by the dry concentrate, defining de fact a unique volume.

As shown in FIG. 10, the dry concentrate bag 100, when made of plastic film/s includes a number of welding lines 130 that define the structure of the bag and the internal housing space 103. The inlet portion 110 may comprise a first inclined welding line 131 and a second inclined welding line 132 both emerging from the inlet port 111 and defining the lower cone portion 112 of the housing space 103. The inlet port 111 is placed at the lowest point of the lower cone portion 112. The first and second inclined welding lines may be straight, as shown in the figures, or alternatively curved: in such a case, the first and second welding lines may follow a concave outward trajectory. If the plastic film is rectangular or squared, both the first inclined welding line 131 and the second inclined welding line 132 define dead volumes having a triangular shape and located out and adjacent to the housing space 103, in particular located towards the bottom of the bag. These dead volumes do not belong to the housing space 103.

Analogously, as shown in FIG. 11, the outlet portion 120 may comprise a third inclined welding line 133 and a fourth inclined welding line 134 both converging to the outlet port 121 defining the upper cone portion 113 of the housing space 103. The third and fourth inclined welding lines may be straight, as shown in the figures, or alternatively curved: in particular the third and fourth welding lines may follow a concave outward trajectory. The outlet port 121 is placed at the highest point of the upper cone portion 113.

If the plastic film is rectangular or squared, both the third inclined welding line 133 and the fourth inclined welding line 134 define dead volumes having a triangular shape and located out and adjacent to the housing space 103, in particular located towards the upper part of the bag. These dead volumes do not belong to the housing space 103.

The dry concentrate bag 100 may further comprise the central portion 140 connecting the inlet portion 110 with the outlet portion 120: in particular the central portion connects the lower cone portion 112 to the upper cone portion 113. The central portion 140 may comprise a fifth welding line 135 and a sixth welding line 136 opposed one to the other with respect to the housing space 103: the fifth welding line 135 is connected to the first inclined welding line 131 of the lower cone portion 112 and to the third inclined welding line 133 of the upper cone portion 113, while the sixth welding line 136 is connected to the second inclined welding line 132 of the lower cone portion 112 and to the fourth inclined welding line 134 of the upper cone portion 113. In particular the first, second, third, fourth, fifth and sixth welding lines delimit and define the housing space 103. In particular the embodiment having two plastic films comprises the first, second, third, fourth, fifth and sixth welding lines. In particular the fifth welding line 135 and the sixth welding line 136 are substantially parallel one another to define a rectangular central portion of the housing space 103.

Clearly, the fifth welding line 135 and the sixth welding line 136 may be absent in case the first inclined welding line 131 and the second inclined welding line 132 both reaches respectively the third inclined welding line 133 and the fourth inclined welding line 134 of the upper cone portion 113.

According to the embodiment comprising a single folded plastic film (see FIGS. 14-17), the central portion 140 may comprise the fifth welding line 135 and a folding line 137 opposed one to the other with respect to the housing space 103. The fifth welding line 135 is connected to the first inclined welding line 131 of the lower cone portion 112 and to the third inclined welding line 133 of the upper cone portion 113, while the folding line 137 is connected to the second inclined welding line 132 of the lower cone portion 112 and to the fourth inclined welding line 134 of the upper cone portion 113. In particular the first, second, third, fourth, fifth welding lines and the folding line 137 delimit and define the housing space 103. In particular the fifth welding line 135 and the folding line 137 are substantially parallel one another to define a rectangular portion of the housing space 103. Notably, the folding line 137 replaces the sixth welding line 136 of the embodiment having two plastic films.

The dry concentrate bag 100 is substantially symmetric with respect to the longitudinal axis A. In particular, the longitudinal axis A of the dry concentrate bag divides symmetrically two portions having trapezoidal shapes. Notably, the trapezoidal shaped portion is delimited by the longitudinal axis A, the first inclined welding line 131, the fifth welding line 135, and the third inclined welding line 133. Correspondingly, the trapezoidal shaped portion may be delimited by the longitudinal axis A, the second inclined welding line 132, the sixth welding line 136 or the folding line 137, and the fourth inclined welding line 134.

As shown in FIG. 11, the first inclined welding line 131 of the lower cone portion 112 defines an angle α with respect to the longitudinal axis A, and the third inclined welding line 133 of the upper cone portion 113 defines a corresponding angle β with respect to the longitudinal axis A: the angle α is smaller than the angle β. Analogously, the second inclined welding line 132 of the lower cone portion 112 defines an angle α′ with respect to the longitudinal axis A, and the fourth inclined welding line 134 of the upper cone portion 113 defines a corresponding angle β′ with respect to the longitudinal axis A. The angle α′ is smaller than the angle β′: in particular, the angle α′ is equal to the angle α, and the angle β′ is equal to the angle β. In particular the angle α and/or a′ is/are smaller than 60°, in particular smaller than 45° and even in more detail smaller than 30°, while the angle β and/or B′ is/are larger than 30°, in particular larger than 45° and even in more detail larger than 60°.

In the additional embodiment of FIG. 10A, the first inclined welding line 131 of the lower cone portion 112 is made of two differently inclined segments 131a and 131b. A first segment 131a emerging from the inlet port 111 defines the angle α with respect to the longitudinal axis A; the second segment 131b emerges from the first segment 131a and defines an auxiliary angle γ that is smaller than the angle α of the first segment. The bag may be symmetric along axis A and consequently also the second inclined welding line 132 may show the respective different segments 132a and 132b defining angles α′ and γ′. Clearly, both or either the first inclined welding line 131 and the second inclined welding line 132 may include more than one, two or three straight segments that define smaller and smaller angles moving from the inlet port towards the upper cone portion. Alternatively, curved welding lines that connect the bottom cone portion and the upper cone portion may be provided alternating with the straight segments.

Notably, the welding lines described above may be made by a heat sealing process, wherein the two plastic films or the first and the second layers 101, 102 of the single plastic film are pressed together and heated up to a sealing welding temperature. The welding lines are fluid tight in order to define a fluid tight housing space 103: in particular the housing space 103 communicates with external environment only through the inlet port 111 and through the outlet port 121. Notably, the plastic films are configured to withstand temperatures up to 80-90 °C.

As visible from FIG. 18A to FIG. 18C, the dry concentrate bag 100 may comprise one or more hanging elements 125 placed on the upper cone portion 113, such as a hole, so that the bag may be hanged on hooks or hangers. In this condition, which corresponds to the standard working condition of the bag 100, the upper cone portion 113 is at a height higher than a corresponding height of the lower cone portion 112. In this condition, the dry concentrate 104 is mostly housed by the lower cone portion.

The dry concentrate bag 100 may further comprise a first filter 150 (see FIGS. 10 and 10A) placed at the outlet port 121, or downstream the outlet port 121, in order to filter all fluid exiting from the outlet port 121 during a working condition (dissolution phase). The first filter 150 is configured to withhold the dry concentrate 104 in solid form and allow the generated fluid to flow through. The generated fluid comprises the substance of the dry concentrate mixed or dissolved in a fluid, such as pure water.

A second filter (or a bottom powder stop) 151 may be provided and placed in correspondence of the inlet port 111 to prevent the dry concentrate, i.e. powder or granulate, to pass through the inlet port 111. Indeed, when recirculating to dissolve only a powder stop is needed, but then direction is switched and the liquid concentrate is pulled through inlet port 111. So for safety, the “powder stop” may be substituted by the second filter 151.

The housing space 103 of the illustrated specific example is configured to house at least 1000 ml of fluid and optionally at least 1250 ml (non-limitative lower volume). This volume values applies in case it is considered to use 1 bag per treatment per day. In any case, multi-therapy concentrates may be used as well and the bags are then typically designed to house 3-5 liters. Notably, the housing space 103 and a dry concentrate 104 quantity housed in the housing space in dry form are such to allow, when in use, a dilution with water to reach a concentration in the liquid concentrate between 15% to 70% w/v.

In one first illustrative embodiment, the dry concentrate 104 may include an osmotic agent, i.e. glucose monohydrate. The dry concentrate comprises at least 80% of glucose monohydrate: optionally the dry concentrate consists of glucose monohydrate.

Alternatively, in another embodiment, the dry concentrate 104 may comprise buffer and electrolyte components. This formulations may be directed to the preparation of medical fluids for peritoneal dialysis or to medical fluids for haemodialysis.

The dry concentrate 104 for PD solutions may comprise at least one substance in the group between NaCl, NaLa, CaCl₂), MgCl₂: in particular at least the 90%, in particular at least the 95%, of the dry concentrate consists of NaCl and NaLa. According to a specific embodiment (non-limiting), the dry concentrate 104 comprises the following substance concentrations:

| Component | Weight percentage |
|---|---|
| NaCl | 53.65% |
| CaCl2 | 1.38% |
| MgCl2 | 0.24% |
| NaLa | 44.73% |

1-22. (canceled)

23. A medical fluid generation apparatus comprising:
a fluid circuit including a main fluid line having an inlet point for receiving water;
a pump configured to operate on the main fluid line to circulate fluid at least in the main fluid line;
a plurality of valves connected to the fluid circuit configurable to define different fluid paths for the fluid inside the fluid circuit,
a supporting structure housing the fluid circuit, the pump and the plurality of valves, and comprising:
a first primary container access being part of the fluid circuit for fluid communication with a primary concentrate container, the first primary container access being in fluid communication with the main fluid line; and
a first auxiliary container access being part of the fluid circuit for fluid communication with an auxiliary concentrate container, the first auxiliary container access being in fluid communication with the main fluid line;
a control unit configured to operate said pump and said plurality of valves; and
a conductivity meter associated with the control unit configured to determine a conductivity of the fluid circulating in the medical fluid generation apparatus,
wherein the medical fluid generation apparatus further comprises at least one of:
a mixing container access being part of the fluid circuit and a mixing container exit being part of the fluid circuit for fluid communication with respectively an inlet of a mixing container and an outlet of the mixing container, the mixing container access and the mixing container exit being respectively in fluid communication with the main fluid line; and
a mixing container having an inlet in fluid communication with the main fluid line and an outlet in fluid communication with the main fluid line,
wherein the fluid circuit comprises:
a medical fluid outlet to provide medical fluid and a medical fluid outlet line for carrying medical fluid to the medical fluid outlet, the medical fluid outlet line being configured for receiving fluid from the main fluid line and being placed downstream the pump;
a mixing container outlet line connecting the outlet of the mixing container to a mixing container outlet junction point on the main fluid line, the mixing container outlet junction point being placed upstream the pump;
a mixing container inlet line connecting a mixing container inlet junction point on the main fluid line to the inlet of the mixing container, the mixing container inlet junction point being placed downstream the pump;
a primary concentrate container outlet line connecting the first primary container access to a primary concentrate container junction point on the main fluid line, the primary concentrate container junction point being placed upstream the pump; and
an auxiliary concentrate container outlet line connecting the first auxiliary container access to an auxiliary concentrate container junction point on the main fluid line, wherein the auxiliary concentrate container junction point is placed upstream the pump,
wherein the conductivity meter is connected to the main fluid line downstream the mixing container outlet junction point, the auxiliary concentrate container junction point, and the primary concentrate container junction point and upstream the mixing container inlet junction point.

24. The medical fluid generation apparatus according to claim 23, further comprising at least one of:
a primary concentrate container having at least one port in fluid communication with the main fluid line, said port being in fluid communication with the first primary container access; and
an auxiliary concentrate container having at least one port in fluid communication with the main fluid line, said port being in fluid communication with the first auxiliary container access.

25. The medical fluid generation apparatus according to claim 24, wherein the primary concentrate container comprises a dry concentrate housed in in the form of a powder or a granulate, the dry concentrate including an osmotic agent and wherein the auxiliary concentrate container comprises liquid concentrate,
wherein the apparatus is for preparing dialysis fluid.

26. The medical fluid generation apparatus according to claim 23, wherein said plurality of valves comprises one or more of:
a mixing container inlet valve connected to the mixing container inlet line;
a mixing container outlet valve connected to the mixing container outlet line;
a medical fluid outlet valve connected to the medical fluid outlet line, the medical fluid outlet is directly connected to the mixing container inlet line to receive the medical fluid flowing in a portion of the mixing container inlet line;
a primary concentrate container outlet valve connected to the primary concentrate container outlet line;
an auxiliary concentrate container outlet valve connected to the auxiliary concentrate container outlet line; and
a water inlet valve placed on the main fluid line upstream said mixing container outlet junction point, auxiliary concentrate container junction point, primary concentrate container junction point, and mixing container inlet junction point.

27. The medical fluid generation apparatus according to claim 23, wherein said fluid paths comprise at least a mixing recirculation path including the mixing container, the outlet of the mixing container, the mixing container outlet line and the mixing container outlet junction point, at least one portion of the main fluid line in which the pump is configured to operate extending between the mixing container outlet junction point and the mixing container inlet junction point, the mixing container inlet junction point and the mixing container inlet line, wherein the plurality of valves are configured to allow a recirculation of the fluid contained in the mixing container through the mixing recirculation path,
wherein the control unit is configured to:
send a liquid concentrate from either the primary concentrate container or the auxiliary concentrate container to the mixing container;
send an amount of water to the mixing container;
circulate, in the mixing recirculation path, a first liquid mixture formed by the liquid concentrate added with water;
perform a main fluid line concentration test step comprising detecting, by means of the conductivity meter, a conductivity of the first liquid mixture flowing through the first mixing recirculation path,
detect a time-resolved variation of said conductivity, said time-resolved variation of the conductivity of the fluid being related to homogeneity of the fluid recirculating in the mixing recirculating path; and
interrupt the circulating step of the first liquid mixture upon the time-resolved variation meeting a variation criterion, the variation criterion including comparing the time-resolved variation of said conductivity with a pre-fixed threshold to determine proper mixing and homogenization of the first liquid mixture in the mixing recirculation path.

28. The medical fluid generation apparatus according to claim 23, further comprising a second conductivity meter associated with the control unit configured to independently determine said conductivity of the fluid circulating in the medical fluid generation apparatus, both said conductivity meter and second conductivity meter sending independent signals to the control unit configured to determine the conductivity of the same fluid to get a back-up measure, said second conductivity meter being placed on the main fluid line, downstream the mixing container outlet junction point, the auxiliary concentrate container junction point, and the primary concentrate container junction point and upstream the mixing container inlet junction point.

29. The medical fluid generation apparatus according to claim 23, further comprising:
a heater controlled by the control unit and configured to heat the fluid circulating in the medical fluid generation apparatus, said heater being placed on the main fluid line, upstream of the pump and/or the conductivity meter; and
a temperature sensor for sensing the temperature of the fluid in the main fluid line, the temperature sensor being placed downstream of the first junction point, the third junction point, the fifth junction point, and the heater and upstream of the mixing container inlet junction point,
wherein the control unit is configured to receive the temperature signal from the temperature sensor and to control the heater at least based on the received temperature signal.

30. The medical fluid generation apparatus according to claim 23, wherein the control unit is configured to control the pump to pump fluid inside the main line in a forward direction from upstream the pump where the inlet point for receiving water, the mixing container outlet junction point, the auxiliary concentrate container junction point, and the primary concentrate container junction point are placed towards downstream the pump where the mixing container inlet junction point is placed, the control unit is configured to never control the pump to pump the fluid in the main line in reverse direction from the mixing container inlet junction point towards any of the inlet point for receiving water, the mixing container outlet junction point, the auxiliary concentrate container junction point, and the primary concentrate container junction point during steps for medical fluid generation.

31. The medical fluid generation apparatus according to claim 23, wherein the fluid circuit further comprises a drain port to discard fluid to a drain and a drain outlet line carrying the fluid from the main line to the drain port, said plurality of valves further comprising a drain valve connected to the drain outlet line.

32. The medical fluid generation apparatus according to claim 23, wherein the fluid circuit comprises a primary concentrate container inlet line connecting a junction point on the main fluid line to the first primary container access, said plurality of valves comprising a primary concentrate container inlet valve connected to the primary concentrate container inlet line.

33. The medical fluid generation apparatus according to claim 23, wherein the fluid circuit comprises an auxiliary concentrate container inlet line connecting a junction point on the main fluid line to the first auxiliary container access, said plurality of valves comprising an auxiliary concentrate container inlet valve connected to the auxiliary concentrate container inlet line.

34. The medical fluid generation apparatus according to claim 33, wherein the auxiliary concentrate container inlet line comprises a common tract with a primary concentrate container inlet line, said common tract starting at the junction point and arriving at a branch where the primary concentrate container inlet line and the auxiliary concentrate container inlet line separate, wherein the auxiliary concentrate container inlet valve and the concentrate container inlet valve are placed downstream said branch.

35. The medical fluid generation apparatus according to claim 23, further comprising a de-aeration chamber configured for allowing air removal and placed on the primary concentrate container outlet line between the first primary container access and the primary concentrate container outlet valve, the de-aeration chamber comprising a vent including a vent line to selectively expel excess air, and a gas valve connected to the vent line to selectively block air passage through the vent line.

36. The medical fluid generation apparatus according to claim 23, wherein the fluid circuit comprises a first concentrate mixing line connecting a primary concentrate container inlet junction point on the main fluid line to the first primary container access, said plurality of valves comprising a first concentrate mixing valve connected to the first concentrate mixing line, wherein the primary concentrate container inlet junction point is placed upstream the pump.

37. The medical fluid generation apparatus according to claim 23, wherein the fluid circuit comprises a second concentrate mixing line connecting an auxiliary concentrate container inlet junction point on the main fluid line to the first auxiliary container access, said plurality of valves comprising a second concentrate mixing valve connected to the second concentrate mixing line,
wherein the auxiliary concentrate container inlet junction point is placed upstream the pump.

38. The medical fluid generation apparatus according to claim 23, wherein the supporting structure comprises an entrance for water defining the inlet point for receiving water.

39. The medical fluid generation apparatus according to claim 23, further comprising a pressure sensor configured for sensing a pressure of the fluid in the main fluid line downstream the pump, wherein the pressure sensor is placed on the mixing container inlet line between the mixing container inlet junction point and a mixing container inlet valve.

40. The medical fluid generation apparatus according to claim 23, further comprising an auxiliary pressure sensor configured for sensing a pressure occurring on the main fluid line upstream the pump, wherein the auxiliary pressure sensor is placed on the mixing container outlet line between the mixing container outlet junction point and a mixing container outlet valve.

41. The medical fluid generation apparatus according to claim 23, further comprising one or more sterilization devices configured to operate on the fluid contained into the fluid circuit, wherein said one or more sterilization devices are configured to operate on the main line and/or the mixing container inlet line and/or the mixing container outlet line and/or on a fluid volume contained into the mixing container.

42. The medical fluid generation apparatus according to claim 23, wherein the mixing container further comprises a vent including a vent line to selectively expel excess air and an air valve to selectively block air passage through the vent line being placed along the vent line, the vent comprising a filter to filter any air passing through the vent line and the air valve being interposed on the vent line between the mixing chamber and the filter.

43. The medical fluid generation apparatus according to claim 23, wherein a first end of the medical fluid outlet line is connected to the mixing container inlet line and a second end of the medical fluid outlet line is connected to the medical fluid outlet, wherein the first end is connected upstream of a mixing container inlet valve to receive the medical fluid flowing in a portion of the mixing container inlet line upstream the mixing container inlet valve.

44. A medical fluid generation apparatus comprising:
a fluid circuit including a main fluid line having an inlet point for receiving water;
a pump configured to operate on the main fluid line to circulate fluid at least in the main fluid line;
a plurality of valves connected to the fluid circuit configurable to define different fluid paths for the fluid inside the fluid circuit,
a supporting structure comprising an entrance for water being the inlet point for receiving water, housing the fluid circuit, the pump and the plurality of valves, and comprising:
a first primary container access being part of the fluid circuit for fluid communication with a primary concentrate container, the first primary container access being in fluid communication with the main fluid line; and
a first auxiliary container access being part of the fluid circuit for fluid communication with an auxiliary concentrate container, the first auxiliary container access being in fluid communication with the main fluid line;
a control unit configured to operate said pump and said plurality of valves, and
a conductivity meter associated with the control unit configured to determine a conductivity of the fluid circulating in the medical fluid generation apparatus;
wherein the medical fluid generation apparatus further comprises at least one of:
a mixing container access being part of the fluid circuit and a mixing container exit being part of the fluid circuit for fluid communication with respectively an inlet of a mixing container and an outlet of the mixing container, the mixing container access and the mixing container exit being respectively in fluid communication with the main fluid line; and
a mixing container having an inlet in fluid communication with the main fluid line and an outlet in fluid communication with the main fluid line,
wherein the fluid circuit comprises:
a medical fluid outlet to provide medical fluid and a medical fluid outlet line for carrying medical fluid to the medical fluid outlet, the medical fluid outlet line being configured for receiving fluid from the main fluid line and being placed downstream the pump;
a mixing container outlet line connecting the outlet of the mixing container to a mixing container outlet junction point on the main fluid line, the mixing container outlet junction point being placed upstream the pump;
a mixing container inlet line connecting a mixing container inlet junction point on the main fluid line to the inlet of the mixing container, the mixing container inlet junction point being placed downstream the pump;
a primary concentrate container inlet line connecting a junction point on the main fluid line to the first primary container access,
a primary concentrate container outlet line connecting the first primary container access to a primary concentrate container junction point on the main fluid line, the primary concentrate container junction point being placed upstream the pump;
an auxiliary concentrate container inlet line connecting a point on the main fluid line to the first auxiliary container access, and
an auxiliary concentrate container outlet line connecting the first auxiliary container access to an auxiliary concentrate container junction point on the main fluid line, wherein the auxiliary concentrate container junction point is placed upstream the pump,
wherein the conductivity meter is connected to the main fluid line downstream the mixing container outlet junction point, the auxiliary concentrate container junction point, and the primary concentrate container junction point and upstream the mixing container inlet junction point.
